(19) United States
(12) Patent Application Publication
(10) Pub. No.: US 2011/0028469 A1
(43) Pub. Date: Feb. 3, 2011

(54) Thiphene-2-Carboxamide Derivatives As Modulators of CCR9 Receptor

(76) Inventors: Eric C. Anderson, Houston, TX (US); Brian Dupre, Houston, TX (US); Robert V. Market, Houston, TX (US); Keith A. Monk, Houston, TX (US); Michael M. Savage, Houston, TX (US)

Correspondence Address: PFIZER INC.;PATENT DEPARTMENT, Bld 114 M/S 9114, EASTERN POINT ROAD, GROTON, CT, 06340, US

(21) Appl. No.: 12/680,763

(22) PCT Filed: Sep. 24, 2008
(86) PCT No.: PCT/IB08/53883

§ 371 Date: Sep. 7, 2010

(30) Foreign Application Priority Data
Oct. 5, 2007 (US) 60997882

(51) Int. Cl. A61K 31/5377; C07D 413/12; C07D 409/12; A61K 31/4025; A61P 35/00; A61P 35/02; A61P 31/18; A61P 3/00; A61P 29/00

(52) U.S. Cl. 514/231.5; 544/146; 548/527; 514/422

(57) ABSTRACT

Provided are compounds of Formula (I) or of Formula (II) that are modulators of CCR9 receptor activity, compositions containing the compounds and methods of use of the compounds and compositions. In certain embodiments, provided are methods for treating or amelioratin diseases associated with modulation of CCR9 receptor activity.

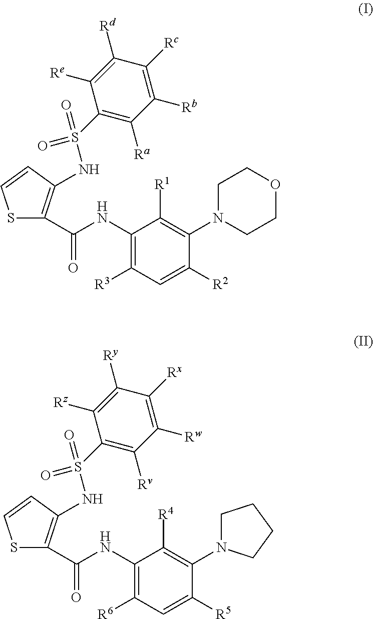

### 1. FIELD

Provided herein are compounds, compositions and methods for treating, preventing or ameliorating conditions associated with CCR9 receptor activity.

### 2. BACKGROUND

Chemokines are chemotactic cytokines that are released by a wide variety of cells and attract various types of immune system cells, such as macrophages, T cells, eosinophils, basophils and neutrophils, to sites of inflammation (reviewed in Schall, Cytokine, 3:165-183 (1991), Schall, et al., Curr. Opin. Immunol., 6:865 873 (1994) and Murphy, Rev. Immun., 12:593-633 (1994)). In addition to stimulating chemotaxis, other changes can be selectively induced by chemokines in responsive cells, including changes in cell shape, transient rises in the concentration of intracellular free calcium ions ([Ca²⁺]), granule exocytosis, integrin up-regulation, formation of bioactive lipids (e.g., leukotrienes) and respiratory burst, associated with leukocyte activation. Thus, the chemokines are early triggers of the inflammatory response, causing inflammatory mediator release, chemotaxis and extravasation to sites of infection or inflammation.

CCR9, a seven transmembrane, G-protein-coupled chemokine receptor was identified as the physiologic receptor for CCL25/thymus-expressed Chemokine (TECK). CCR9 is mainly expressed in thymocytes and T lymphocytes from the small intestine and colon. CCL25/TECK is predominantly expressed in the thymus and small intestine. Studies have shown that CCR9 mediates chemotaxis in response to CCL25/TECK and is likely to play an important role in regulating the trafficking of developing T cells within the thymus and be critical for the development, homeostasis, and/or function of mucosal T lymphocytes.

It has been shown that CCR9+ lymphocytes are markedly elevated in peripheral blood lymphocytes in patients with small bowel Crohn's or celiac disease. TECK expression is altered in an inflamed small bowel, being intensely expressed in a patchy distribution in crypt epithelial cells in proximity to lymphocytic infiltrates (Papadakis et al. Gastroenterology, 2001, 121:246-254). In mouse models, neutralization of TECK inhibits homing of CD8+ T cells to the IEL (intraepithelial lymphocyte) compartment. This directly demonstrates that CCL25 and CCR9 function in recruiting effector lymphocytes to the small intestinal epithelium following their activation in gut-associated lymphoid tissue (GALT).

Targeting CCL25/TECK and/or CCR9 may provide a way to selectively modulate small-intestinal immune responses as suggested by the fact that activated CCR9(+) CD8alphabeta(+) lymphocytes selectively localized to the small-intestinal mucosa, and in vivo neutralization of CCL25/TECK reduced the ability of these cells to populate the small-intestinal epithelium. These results demonstrate an important role for chemokines in the localization of T lymphocytes to the small-intestinal mucosa. (Svensson et al., J. Clin. Invest., 2002, 110:1113-21). CCR9+ gut-homing lymphocytes have also been implicated in primary sclerosing cholangitis, a chronic liver disease that is a common complication of inflammatory bowel disease (Eksteen et al., J. Exp. Med., 2004, 200:1511-1517).

CCR9 receptor expression on human eosinophils from peripheral blood and bronchoalveolar lavage fluid after segmental antigen challenge was reported recently (Liu et al, J Allergy Clin Immunol 2003 September;112(3):556-62). Studies by Singh et al. (Clinical Cancer Research, 2004, 10, 8743-8750) suggest that the expression and activation of CCR9 affect cancer cell migration, invasion, and MMP expression, which together may affect prostate cancer metastasis. In a similar fashion, functional CCR9 has been detected on the surface of small intestinal melanoma (Letsch et al., 2004 J. Invest. Dermatol. 122:685-690).

CCR9 was also found to be selectively expressed on T-ALL CD4+ T cells and moderately expressed on T-CLL CD4+ T cells. CCL25/TECK selectively induced T-ALL CD4+ T cell chemotaxis and adhesion (Qiuping et al., Cancer Res. 2003 Oct. 1; 63(19):6469-77. Annels et al., Blood 2003). A recent study also demonstrates an increase in the expression of CCR9 on peripheral blood gammadelta T cells in individuals having HIV-1 infection (Poles et al., J Virol. 2003 October; 77(19):10456-67).

Because of the involvement of the CCR9 receptor in a variety of diseases, there is a continuing need for compounds that modulate the binding or function of various chemokines to the CCR9 receptor.

### 3. SUMMARY

Provided herein are compounds that are modulators of a CCR9 receptor, pharmaceutical compositions containing the compounds and methods of use thereof In certain embodiments, the compounds for use in the compositions and methods provided herein are of formula I:

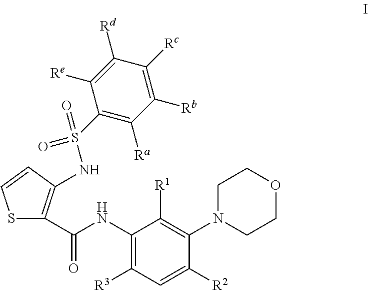

or pharmaceutically acceptable derivatives thereof, wherein the variables are chosen such that the resulting compounds show activity as CCR9 modulators.

In certain embodiments, the compounds for use in the compositions and methods provided herein are of formula II:

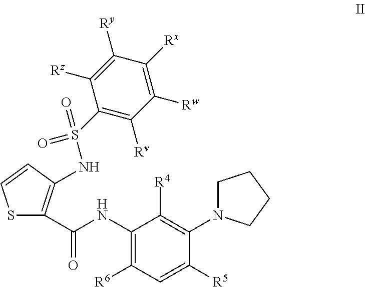

or pharmaceutically acceptable derivatives thereof, wherein the variables are chosen such that the resulting compounds show activity as CCR9 modulators. In one embodiment, the compounds of Formula I or II are CCR9 receptor antagonists.

Pharmaceutical compositions containing a compound of Formula I or II and a pharmaceutically acceptable carrier are provided herein. Also provided are methods for treating, preventing, or ameliorating one or more symptoms of CCR9 receptor mediated diseases by administering the compounds and compositions provided herein.

In certain embodiments, provided herein are methods for modulating an activity of a CCR9 receptor by contacting the receptor with a compound or composition provided herein. In one embodiment, provided herein are methods for antagonizing an action of a CCR9 receptor by contacting the receptor with a compound or composition provided herein. In other embodiments, provided herein are methods for treatment, prevention, or amelioration of one or more symptoms of diseases or conditions associated with CCR9 receptor activity, including, but not limited to inflammatory bowel disease, including Crohn's disease and ulcerative colitis, celiac disease and other forms of intestinal inflammation, including celiac sprue and gluten-sensitive enteropathy; primary sclerosing cholangitis; HIV; as well as various cancers, including, prostate cancer, leukemia, and small intestinal melanoma.

### 4. DETAILED DESCRIPTION

4.1 Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein “subject” is an animal, such as a mammal, including human, such as a patient.

The terms “CCR9 receptor mediated disease, or “CCR9 receptor mediated condition”, as used herein, mean any disease or other deleterious condition or state in which CCR9 receptor is known to play a role. Such diseases or conditions include, without limitation, Crohn's disease, ulcerative colitis, celiac disease, primary sclerosing cholangitis, HIV, prostate cancer, leukemia, small intestinal cancer or melanoma.

As used herein, biological activity refers to the in vivo activities of a compound or physiological responses that result upon in vivo administration of a compound, composition or other mixture. Biological activity, thus, encompasses therapeutic effects and pharmacokinetic behaviour of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating inflammation.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, the IC₅₀ refers to an amount, concentration or dosage of a particular test compound that achieves a 50% inhibition of a maximal response in an assay that measures such response.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter enzymatic and biological activities of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, the nomenclature alkyl, alkoxy etc. is used as is generally understood by those of skill in this art.

As used herein, alkyl carbon chains, if not specified, contain from 1 to 20 carbons, or 1 to 16 carbons, and are straight or branched. Exemplary alkyl groups herein include, but are not limited to, methyl, ethyl, propyl, isopropyl, isobutyl, n-butyl, sec-butyl, tert-butyl, isopentyl, neopentyl, tert-pentyl, isohexyl. As used herein, lower alkyl refers to carbon chains having from about 1 or about 2 carbons up to about 6 carbons.

As used herein, “halo”, “halogen” or “halide” refers to F, Cl, Br or I.

As used herein, “haloalkyl” refers to an alkyl group in which one or more of the hydrogen atoms are replaced by halogen. Such groups include, but are not limited to, chloromethyl, trifluoromethyl and 1 chloro 2 fluoroethyl.

As used herein, “haloalkoxy” refers to RO in which R is a haloalkyl group.

Where the number of any given substituent is not specified (e.g., “haloalkyl”), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

4.2 Compounds

In certain embodiments, the compounds provided herein have enhanced tolerability as compared to similar compounds known in the art. Such enhanced tolerability is manifested through alteration of the pharmacokinetic profile of the compounds. The pharmacokinetic profile is based on a number of factors, including, but not limited to, bioavailability, in vivo half-life and in vivo efficacy. In certain embodiments, the compounds provided herein have improved properties including but not limited to in vitro or in vivo activity in modulation of a CCR9 receptor activity, stability and receptor-selectivity as compared to similar compounds known in the art.

In certain embodiments, the compounds for use in the compositions and methods provided herein are of Formula I:

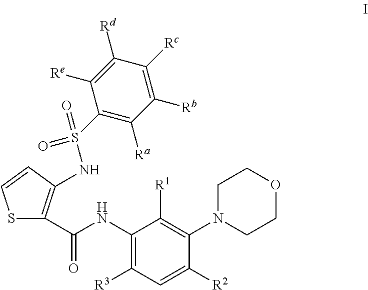

or pharmaceutically acceptable derivatives thereof,

wherein R¹ and R³ are each alkyl; R² is selected from alkyl and halo; and

R^{a}, R^{b}, R^{c}, R^{d} and R^{e} are each independently selected from hydrogen, halo, alkyl, alkoxy, haloalkyl and haloalkoxy, with a proviso that when R^{a}, R^{b}, R^{c}, R^{d} and R^{e} are each hydrogen then at least one of R¹, R² or R³ is other than methyl.

In another embodiment, the compound of Formula I or a pharmaceutically acceptable derivative thereof is such that R¹ and R³ are each alkyl, R² is halo and R^{a}, R^{b}, R^{c}, R^{d} and R^{e} are each independently selected from hydrogen, halo, alkyl, alkoxy, haloalkyl and haloalkoxy. In another embodiment, R¹ and R³ are each alkyl, R² is halo and R^{a}, R^{b}, R^{c}, R^{d} and R^{e} are each hydrogen. In another embodiment, R¹, R² and R³ are each alkyl; and R^{a}, R^{b}, R^{c}, R^{d} and R^{e} are each independently selected from hydrogen, halo, alkyl, alkoxy, haloalkyl and haloalkoxy, such that at least one of R^{a}, R^{b}, R^{c}, R^{d} or R^{e} is other than hydrogen. In another embodiment, R¹ and R³ are each alkyl, R² is halo or alkyl; and R^{a}, R^{b}, R^{c}, R^{d} and R^{e} are each independently selected from halo, alkyl, alkoxy, haloalkyl and haloalkoxy.

In certain embodiments, the compounds of Formula I are selected such that at least one of R^{a}, R^{b}, R^{c}, R^{d} and R^{e} is other than hydrogen when R² is alkyl.

In certain embodiments, R¹ is methyl. In certain embodiments, R³ is methyl.

In certain embodiments, R² is halo. In certain embodiments, R² is chloro or fluoro. In certain embodiments, R² is methyl.

In certain embodiments, R¹ R² and R³ are each alkyl. In one embodiment, R¹ R² and R³ are each methyl.

In certain embodiments, R^{a}, R^{b}, R^{c}, R^{d} and R^{e} are each independently hydrogen, fluoro, chloro, methyl, ethyl, t-butyl, methoxy, trifluoromethoxy or trifluoromethyl. In certain embodiments, R^{a}, R^{b}, R^{c}, R^{d} and R^{e} are each independently hydrogen, fluoro, chloro, methyl, ethyl, t-butyl, methoxy, trifluoromethoxy or trifluoromethyl such that at least one of R^{a}, R^{b}, R^{c}, R^{d} or R^{e} is other than hydrogen.

In one embodiment, R^{c} is hydrogen, halo, alkyl, alkoxy, haloalkyl or haloalkoxy. In one embodiment, R^{c} is halo, alkyl, alkoxy, haloalkyl or haloalkoxy. In one embodiment, R^{c} is halo or alkyl.

In one embodiment, R^{c} is hydrogen, fluoro, chloro, methyl, ethyl, t-butyl, methoxy, trifluoromethoxy or trifluoromethyl. In one embodiment, R^{c} is fluoro, chloro, methyl, ethyl, t-butyl, methoxy, trifluoromethoxy or trifluoromethyl. In one embodiment, R^{c} is fluoro, chloro or methyl. In one embodiment, R^{c} is fluoro. In one embodiment, R^{c} is methyl.

In one embodiment, R^{c} is fluoro, chloro, methyl, ethyl, t-butyl, methoxy, trifluoromethoxy or trifluoromethyl and R^{a}, R^{b}, R^{d} and R^{e} are each selected from hydrogen, chloro, fluoro and methyl.

In certain embodiments, R^{a}, R^{b}, R^{d} and R^{e} are each hydrogen, and R^{c} is selected from halo, alkyl, alkoxy, haloalkyl and haloalkoxy. In certain embodiments, R^{a}, R^{b}, R^{d} and R^{e} are each hydrogen, and R^{c} is selected from halo and alkyl.

In one embodiment, R^{c} is fluoro, chloro or methyl. In one embodiment, R^{c} is fluoro.

In one embodiment, the compound is of formula Ia:

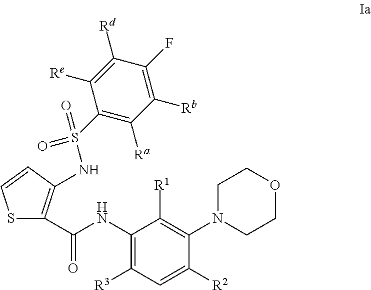

or a pharmaceutically acceptable derivative thereof, wherein the variables are as described elsewhere herein.

In one embodiment, the compound is of formula Ib:

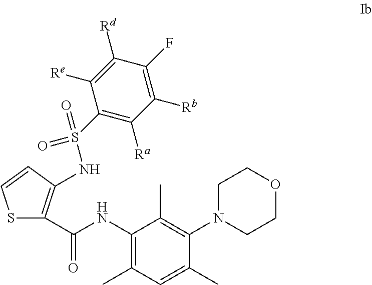

or a pharmaceutically acceptable derivative thereof, wherein the variables are as described elsewhere herein.

In one embodiment, the compound is of formula Ic:

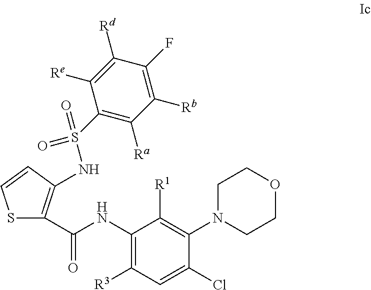

or a pharmaceutically acceptable derivative thereof, wherein the variables are as described elsewhere herein.

In one embodiment, the compound is of formula Id:

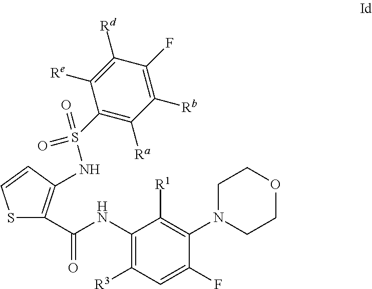

or a pharmaceutically acceptable derivative thereof, wherein the variables are as described elsewhere herein.

In one embodiment, the compound is of formula Ie:

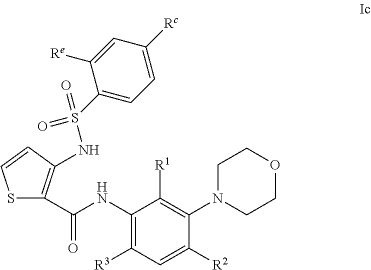

or a pharmaceutically acceptable derivative thereof, wherein the variables are as described elsewhere herein.

In one embodiment, the compound is of formula If:

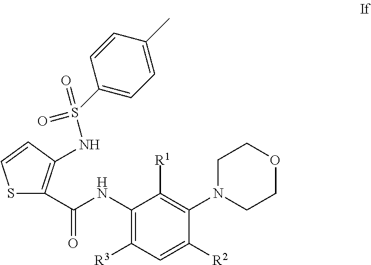

or a pharmaceutically acceptable derivative thereof, wherein the variables are as described elsewhere herein.

In one embodiment, the compound is of formula Ig:

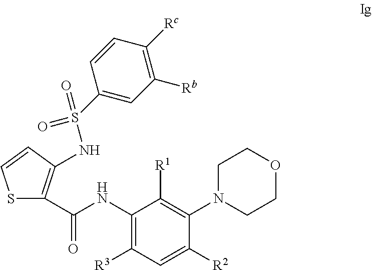

or a pharmaceutically acceptable derivative thereof, wherein the variables are as described elsewhere herein.

In one embodiment, the compound is of formula Ih:

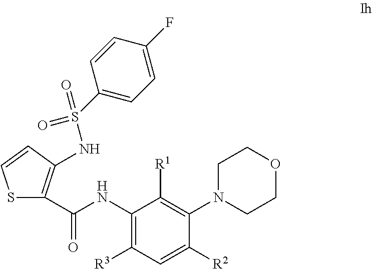

or a pharmaceutically acceptable derivative thereof, wherein the variables are as described elsewhere herein.

In one embodiment, the compound is of formula Ii:

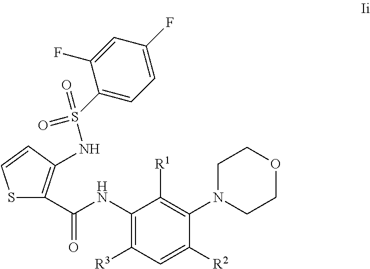

or a pharmaceutically acceptable derivative thereof, wherein the variables are as described elsewhere herein.

In one embodiment, the compound is of formula Ij:

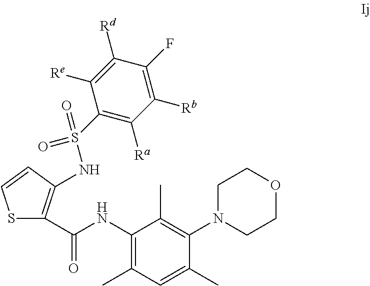

or a pharmaceutically acceptable derivative thereof, wherein the variables are as described elsewhere herein.

In one embodiment, the compound is of formula Ik:

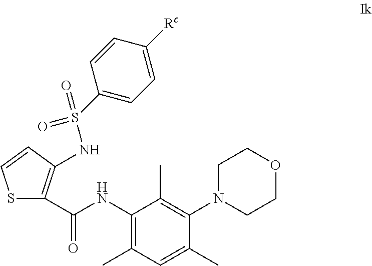

or a pharmaceutically acceptable derivative thereof, wherein the variables are as described elsewhere herein.

In one embodiment, the compound is selected from:

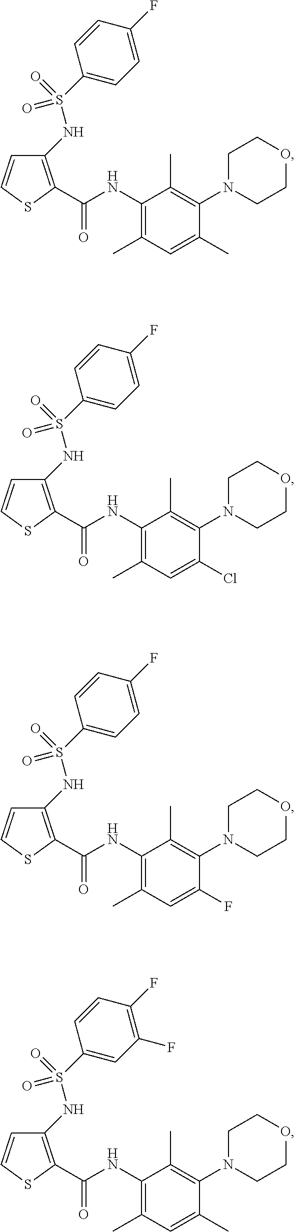

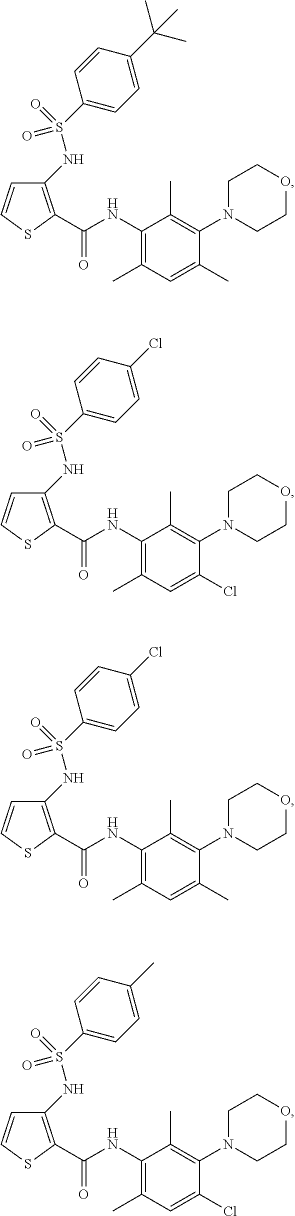

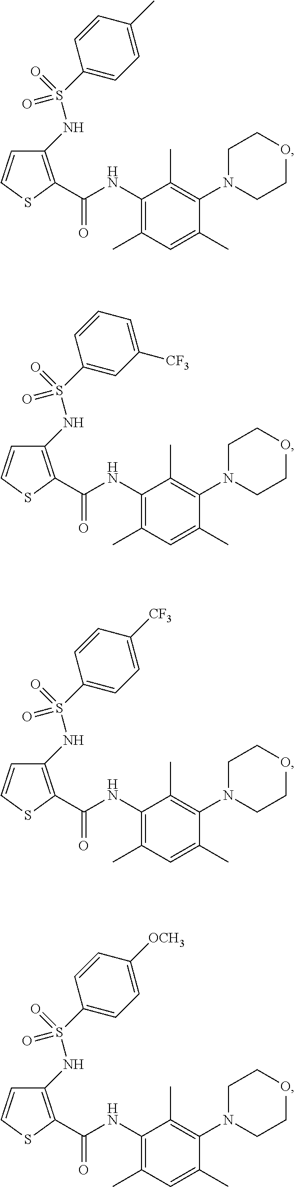

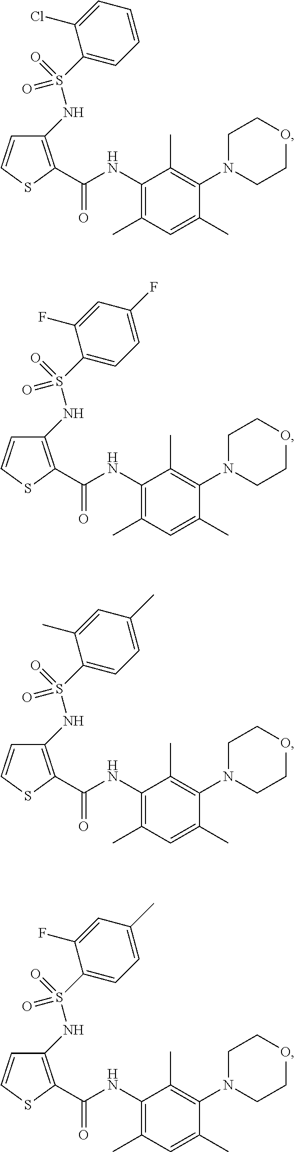

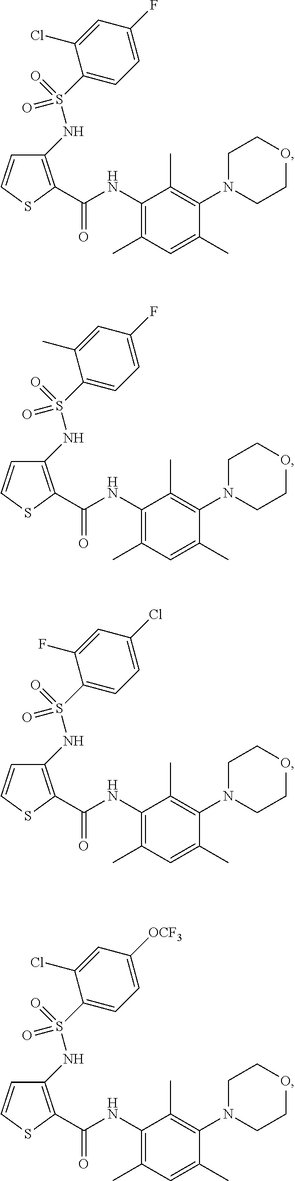

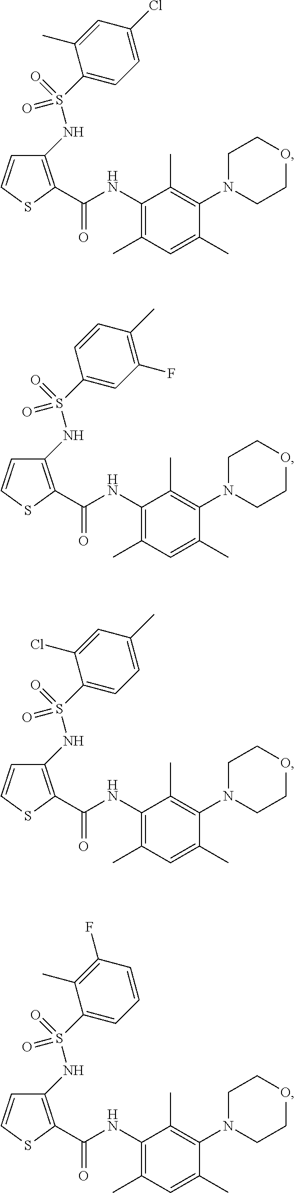

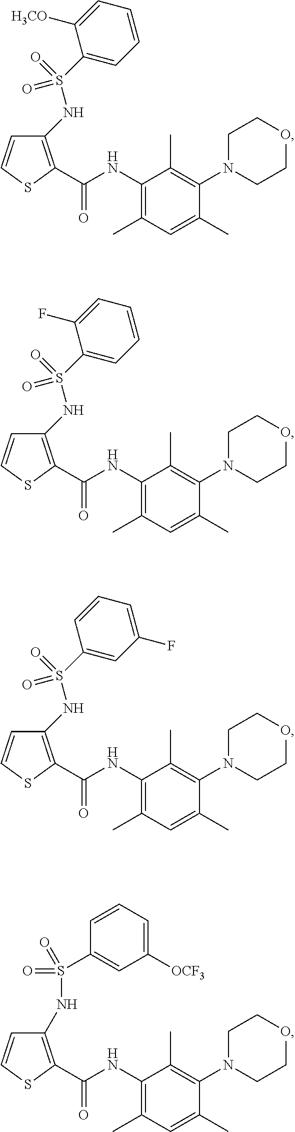

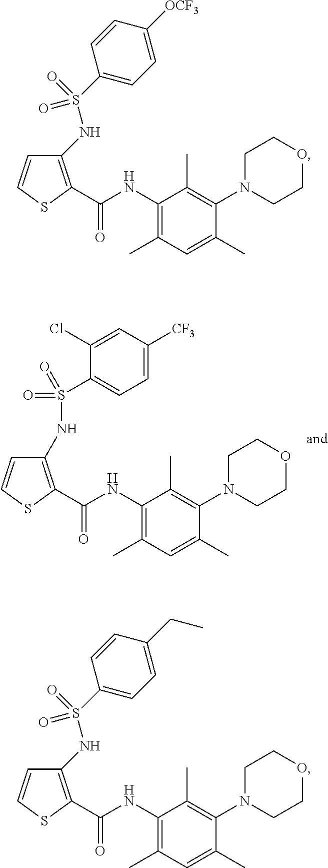

or a pharmaceutically acceptable derivative thereof.

In one embodiment, the compound is of formula II:

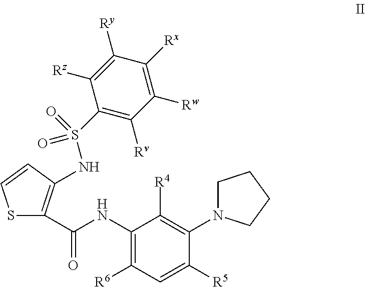

or pharmaceutically acceptable derivatives thereof,

wherein R⁴ is alkyl; R⁵ and R⁶ are each independently selected from halo and alkyl such that at least one of R⁵ or R⁶ is halo; and

R^{v}, R^{w}, R^{x}, R^{y} and R^{z} are each independently selected from hydrogen, halo and alkyl.

In certain embodiments, R⁴ is alkyl. In certain embodiments, R⁴ is methyl.

In certain embodiments, R⁵ is halo. In certain embodiments, R⁵ is chloro or fluoro. In certain embodiments, R⁵ is alkyl. In certain embodiments, R⁵ is methyl.

In certain embodiments, R⁶ is halo. In certain embodiments, R⁶ is chloro. In certain embodiments, R⁶ is alkyl. In certain embodiments, R⁶ is methyl.

In one embodiment, at least one of R⁵ and R⁶ is chloro. In certain embodiments, at least one of R⁵ and R⁶ is halo and the other is methyl. In one embodiment, at least one of R⁵ and R⁶ is chloro and the other is methyl. In another embodiment, R⁵ is fluoro and R⁴ and R⁶ are methyl.

In certain embodiments, R^{v}, R^{w}, R^{x}, R^{y} and R^{z} are each independently hydrogen, fluoro, chloro or methyl. In certain embodiments, R^{v}, R^{w}, R^{y} and R^{z} are each hydrogen. In certain embodiments, R^{v}, R^{w}, R^{y} and R^{z} are each hydrogen and R^{x} is halo.

In one embodiment, R^{x} is hydrogen. In one embodiment, R^{x} is halo. In one embodiment, R^{x} is fluoro or chloro. In one embodiment, R^{x} is alkyl. In one embodiment, R^{x} is methyl.

In one embodiment, R^{w} is halo. In one embodiment, R^{w} is fluoro. In one embodiment, R^{v}, R^{x}, R^{y} and R^{z} are each hydrogen and R^{w} is fluoro.

In one embodiment, the compound is of formula IIa:

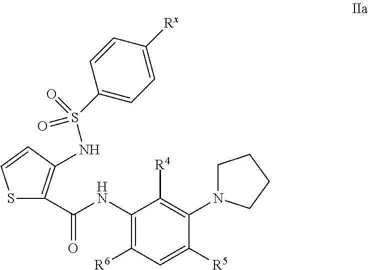

or a pharmaceutically acceptable derivative thereof, wherein the variables are as described elsewhere herein.

In one embodiment, the compound is of formula IIb:

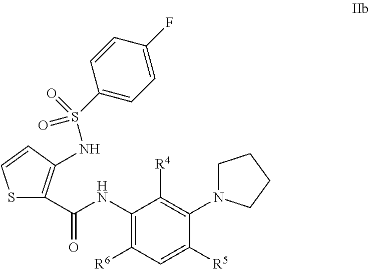

or a pharmaceutically acceptable derivative thereof, wherein the variables are as described elsewhere herein.

In one embodiment, the compound is of formula IIc:

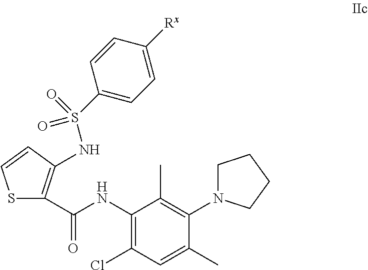

or a pharmaceutically acceptable derivative thereof, wherein the variables are as described elsewhere herein.

In one embodiment, the compound is of formula IId:

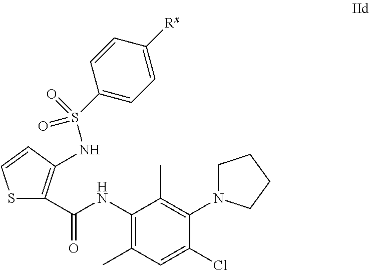

or a pharmaceutically acceptable derivative thereof, wherein the variables are as described elsewhere herein.

In one embodiment, the compound is of formula IIe:

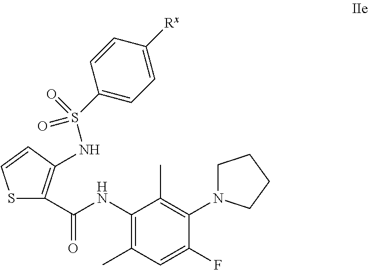

or a pharmaceutically acceptable derivative thereof, wherein the variables are as described elsewhere herein.

In one embodiment, the compound is selected from:

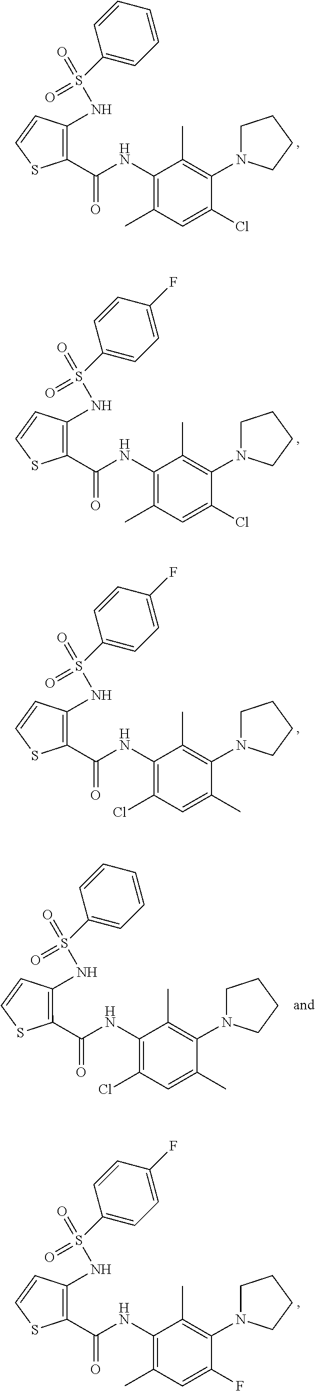

or a pharmaceutically acceptable derivative thereof.

In one embodiment, the compound provided herein is a pharmaceutically acceptable salt of the compound of Formula I or II. In one embodiment, the compound provided herein is a sodium salt of the compound of Formula I or II.

In certain embodiments, the compounds provided herein have improved properties over the compounds previously disclosed. Such properties include one or more of the following: activity in modulation of CCR9 receptor activity, selectivity for CCR9 receptors, pharmacokinetic properties, toxicity, bioavailability and others.

4.2.1 Preparation of the Compounds

The compounds provided herein can be prepared by routine chemical reactions known to one of skill in the art. General schemes for preparation of exemplary compounds are illustrated below:

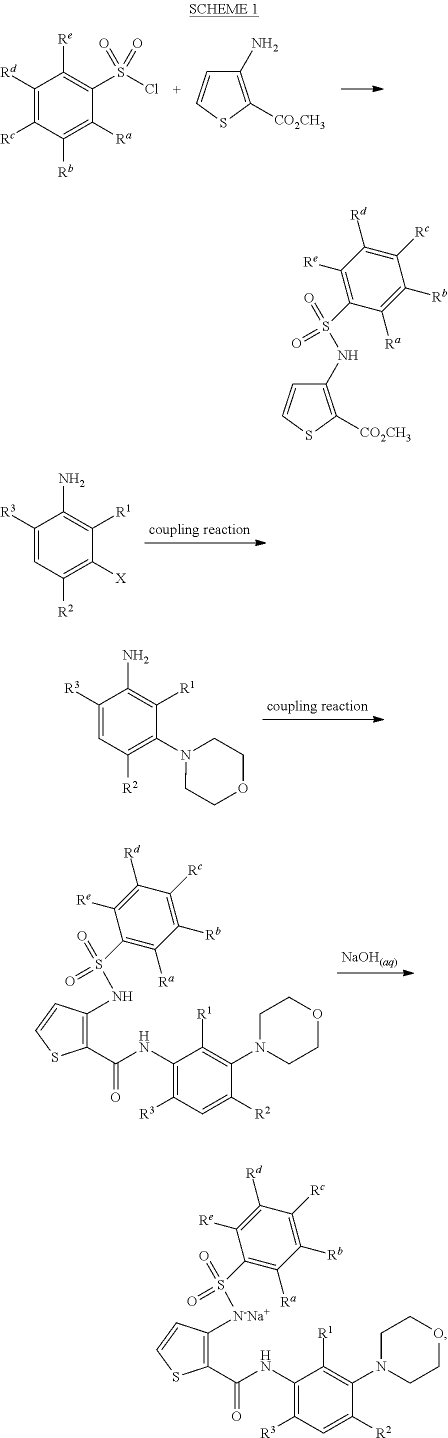

where X is halogen, and R¹-R³ and R^{a}-R^{e} are selected from groups described above.

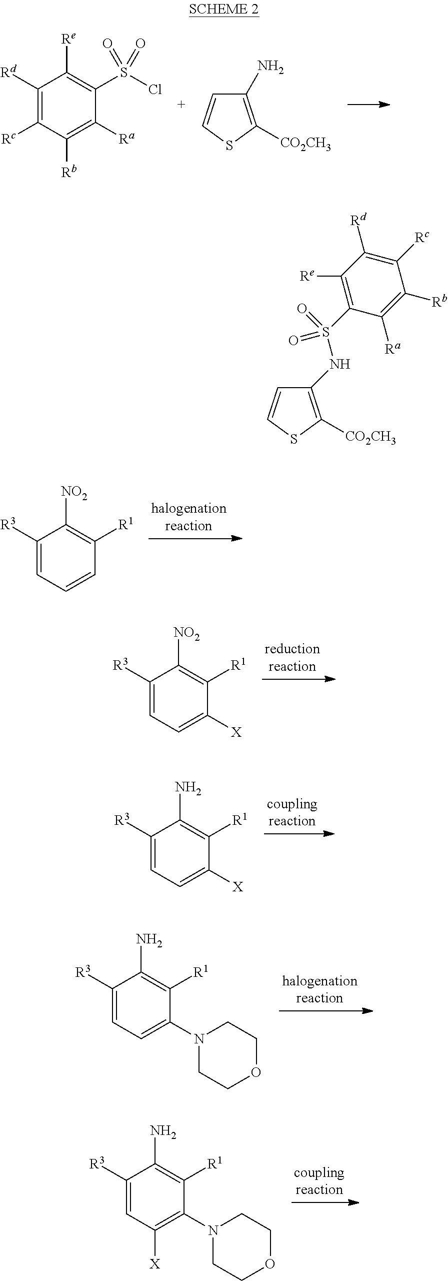

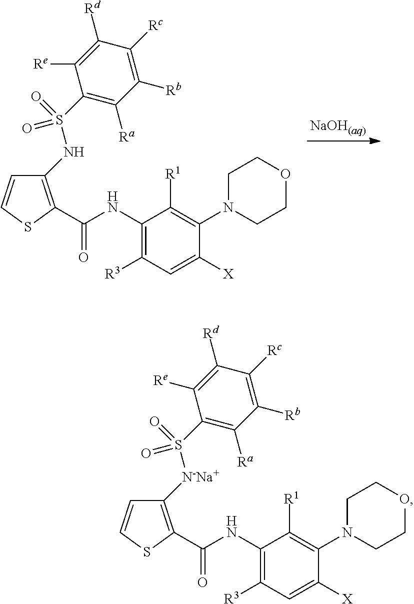

where X is halogen, and R¹, R³ and R^{a}-R^{e} are selected from groups described above. above.

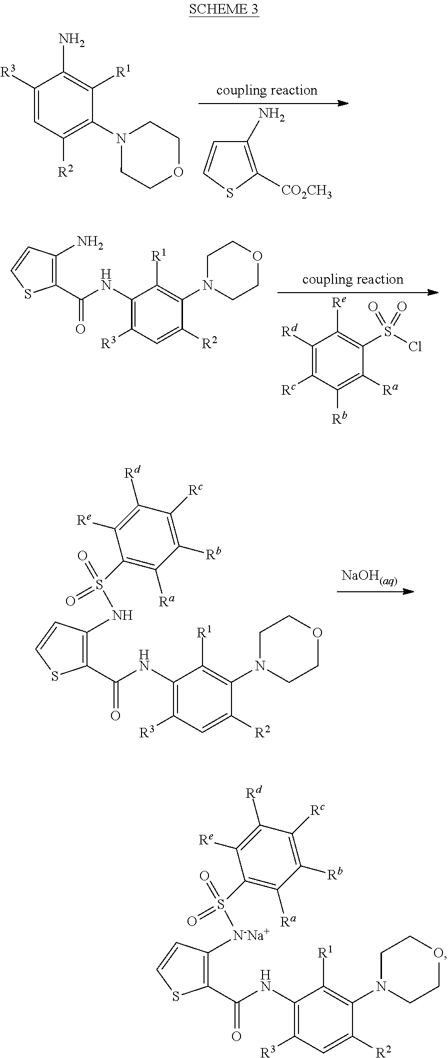

where R¹-R³ and R^{a}-R^{e} are selected from groups described above.
Suitable reagents for coupling, protection and deprotection are known to one of skill in the art.

4.3 Formulation of Pharmaceutical Compositions

The pharmaceutical compositions provided herein contain therapeutically effective amounts of one or more of compounds provided herein that are useful in the preventing, treating, or ameliorating a CCR9-modulated disease or one or more of the symptoms thereof. The pharmaceutical compositions comprise one or more compounds provided herein in a pharmaceutically acceptable vehicle, carrier, diluent, or excipient, or a mixture thereof.

In one embodiment, provided herein are pharmaceutical compositions in modified release dosage forms, which comprise a compound of Formula I or II or a pharmaceutically acceptable derivative thereof, and one or more release controlling excipients as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients.

Further provided herein are pharmaceutical compositions in enteric coated dosage forms, which comprise a compound of Formula I or II or a pharmaceutically acceptable derivative thereof, and one or more release controlling excipients for use in an enteric coated dosage form. The pharmaceutical compositions may also comprise non-release controlling excipients.

Additionally provided are pharmaceutical compositions in a dosage form that has an instant releasing component and at least one delayed releasing component, and is capable of giving a discontinuous release of the compound in the form of at least two consecutive pulses separated in time from 0.1 up to 24 hours. The pharmaceutical compositions comprise a compound of Formula I or II or a pharmaceutically acceptable derivative thereof, and one or more release controlling and non-release controlling excipients, such as those excipients suitable for a disruptable semi-permeable membrane and as swellable substances.

In certain embodiments, provided herein are pharmaceutical compositions in a dosage form for oral administration to a subject, which comprise a compound of Formula I or II or a pharmaceutically acceptable derivative thereof, and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In one embodiment, the pharmaceutical compositions herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampouls, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The compound of Formula I or II provided herein may be administered alone, or in combination with one or more other compounds provided herein, one or more other active ingredients. The pharmaceutical compositions that comprise a compound provided herein may be formulated in various dosage forms for oral, parenteral, and topical administration. The pharmaceutical compositions may also be formulated as modified release dosage forms, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

A. Oral Administration

The pharmaceutical compositions provided herein may be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure that the tablet remains intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Micellar dosage forms can be prepared as described in U.S. Pat. No. 6,350,458.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as antacids, proton pump inhibitors, and H₂-receptor antagonists.

B. Parenteral Administration

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including alpha-cyclodextrin, beta-cyclodextrin, hydroxypropyl-beta-cyclodextrin, sulfobutylether-beta-cyclodextrin, and sulfobutylether 7-beta-cyclodextrin (CAPTISOL®, CyDex, Lenexa, Kans.).

The pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

C. Topical Administration

The pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon bases, including such as lard, benzoinated lard, olive oil, cottonseed oil, and other oils, white petrolatum; emulsifiable or absorption bases, such as hydrophilic petrolatum, hydroxystearin sulfate, and anhydrous lanolin; water-removable bases, such as hydrophilic ointment; water-soluble ointment bases, including polyethylene glycols of varying molecular weight; emulsion bases, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The pharmaceutical compositions provided herein may be micronized to a size suitable for delivery by inhalation, such as 50 micrometers or less, or 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

The pharmaceutical compositions provided herein for topical administration may be formulated to be immediate release or modified release, including delayed-, sustained-, pulsed-, controlled-, targeted, and programmed release.

D. Modified Release

The pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile- or pulsed-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

1. Matrix Controlled Release Devices

The pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In another embodiment, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, hydrophilic polymers, such as ethyl cellulose, cellulose acetate, crospovidone, and cross-linked partially hydrolyzed polyvinyl acetate,; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients in the compositions.

The pharmaceutical compositions provided herein in a modified release dosage form may be prepared by methods known to those skilled in the art, including direct compression, dry or wet granulation followed by compression, melt-granulation followed by compression.

2. Osmotic Controlled Release Devices

The pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” include, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphate, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-tolunesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiment, the pharmaceutical compositions provided herein are formulated as ESC controlled-release dosage form, which comprises an osmotic membrane that coats a core comprising the active ingredient(s), hydroxylethyl cellulose, and other pharmaceutically acceptable excipients.

3. Multiparticulate Controlled Release Devices

The pharmaceutical compositions provided herein in a modified release dosage form may be fabricated as a multiparticulate controlled release device, which comprises a multiplicity of particles, granules, or pellets, ranging from about 10 μm to about 3 mm, about 50 μm to about 2.5 mm, or from about 100 μm to 1 mm in diameter. Such multiparticulates may be made by the processes know to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

4. Targeted Delivery

The pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

4.3.1 Articles of Manufacture

The compounds or pharmaceutically acceptable derivatives can be packaged as articles of manufacture containing packaging material, a compound or pharmaceutically acceptable derivative thereof provided herein, which is used for treatment, prevention or amelioration of one or more symptoms associated with CCR9 activity, and a label that indicates that the compound or pharmaceutically acceptable derivative thereof is used for treatment, prevention or amelioration of one or more symptoms of CCR9 receptor mediated diseases.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated.

4.4 Evaluation of the Activity of the Compounds

The CCR9 antagonist activity of the compounds provided herein can be demonstrated by methods known to one of skill in the art. Exemplary methods are described in US Publication Nos. US2004/0180892 and US 2005/0049286, which are incorporated herein by reference. An exemplary assay for determining CCR9 antagonist activity of the compounds provided herein is CCR9 FLIPR/FlexStation Assay. In certain embodiments, the following protocol is used for the assay:

CCR9 FLIPR/FlexStation Assay Protocol

Calcium assay in FLIPR/FlexStation determines inhibitors of TECK induced calcium mobilization in CCR9-Flp-CHO cells that stably over express human CCR9 receptor. CCR9-Flp-CHO cells are seeded at 25,000 cells/well in a clear bottom, black wall 96-well plate (Greiner #655090) one day prior to assay. Cells are grown in a tissue culture incubator at 37° C. with 5% CO₂ for 18 to 24 hours.

Wash buffer and dye loading buffer are prepared fresh each time the assay is performed. Wash buffer is prepared according to the following protocol: 20 ml 10×HBSS (Invitrogen/Gibco #14065-056), 4 ml 1 M HEPES (Sigma H3784), 174 ml sterile deionized water; then add 140 mg probenecid (Sigma P8761) dissolved in 2 ml 1 M NaOH (Fisher S318) to solution and pH to 7.4. This wash buffer contains 1×HBSS, 20 mM HEPES and 2.5 mM probenecid. For one 96-well plate, dye loading buffer is prepared as following: 11 ml wash buffer, 44 μl Fluo-4/pluoronic acid mix (prepared from 22 μL aliquot of 2 mM Fluo-4 (Molecular Probes F14202, 1 mg/tube)+22 μl 20% pluronic F-127 (Molecular Probes P3000MP).

Cells are loaded with dye according to the protocol below:
1. Prepare wash buffer with 1×HBSS/HEPES at room temperature
2. Prepare loading buffer (keep in dark)
3. Aspirate culture media and wash with 100 μl wash buffer per well×1
4. Add 100 μl dye loading buffer to each well
5. Incubate at 37° C. for 30 minutes
6. Aspirate loading buffer
7. Wash with 100 μl wash buffer per well×3
8. Add 100 μl wash buffer per well
9. Incubate at 37° C. for 30 minutes
10. Assay plate with FLIPR or FlexStation

10 mM stock compounds in DMSO are prepared and diluted in DMSO to 1 mM. Compounds are diluted in wash buffer to make 8 point series dilutions containing same final concentration of DMSO (1%). Compounds are tested in duplicate wells for each point. Ligand rhTECK (R&D Systems 334-TK) was diluted to 6× of its EC70 with wash buffer. Appropriate amount of 6× ligand is added to each well. Data is analyzed using XLfit3 software to calculate IC₅₀ value of antagonist activity for each compound.

4.5 Methods of Treatment and Prevention

In certain embodiments, provided herein are methods for modulating an activity of CCR9 receptor by contacting the receptor with a compound or composition provided herein. In one embodiment, provided herein are methods for antagonizing an action of CCR9 receptor by contacting the receptor with a compound or composition provided herein.

In other embodiments, provided herein are methods for treatment, prevention, or amelioration of one or more diseases or conditions associated with CCR9 receptor activity, including, but not limited to inflammatory bowel disease, including Crohn's disease and ulcerative colitis, celiac disease and other forms of intestinal inflammation, including celiac sprue and gluten-sensitive enteropathy; primary sclerosing cholangitis; HIV; as well as various cancers, including, prostate cancer, leukemia, and small intestinal melanoma.

4.5.1 Combination Therapy with a Second Active Agent

The compounds provided herein may be administered as the sole active ingredient or in combination with other active ingredients. Other active ingredients that may be used in combination with the compounds provided herein include but are not limited to, compounds known to treat diseases associated with CCR9 receptor modulation or compounds known to modulate CCR9 receptor activity. Examples of such compounds include, but are not limited to antihistamines, corticosteroids, β2-agonists, steroid receptor modulators, anticholinergic compounds, immunomodulators, bronchdilators, leukotriene modifiers, COX-2 inhibitors and anti-inflammatory compounds.

Administration of the active ingredient combination may take place either by separate administration of the active ingredients to the patient or in the form of combination products in which a plurality of active ingredients are present in one pharmaceutical preparation.

It will be appreciated that every suitable combination of the compounds provided herein with one or more of the aforementioned compounds and optionally one or more further pharmacologically active substances is contemplated herein.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### 5. Examples

Certain embodiments of the claimed subject matter are illustrated by the following non-limiting examples.

### Example 1

### Preparation of N-[4-Fluoro-2,6-dimethyl-3-(pyrrolidin-1-yl)phenyl]-3-(4-fluorophenylsulfonamido)thiophene-2-carboxamide, Sodium Salt (7)

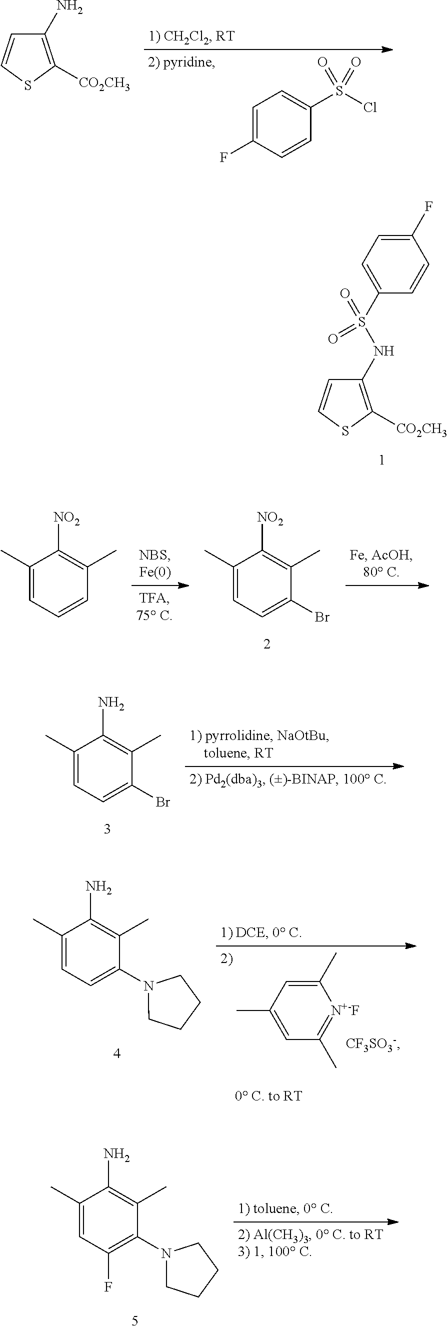

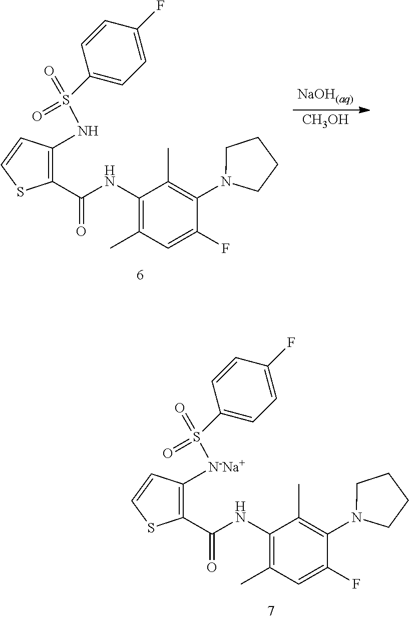

### I. Methyl 3-(4-fluorophenylsulfonamido)thiophene-2-carboxylate (1)

To a solution of methyl 3-aminothiophene-2-carboxylate (2.00 g; 12.72 mmol) in dichloromethane (25 mL), was added pyridine (2.17 mL; 25.4 mmol) and 4-fluorobenzene-1-sulfonyl chloride (2.47 g; 12.72 mmol). After stirring overnight at room temperature, the reaction was quenched by addition of aqueous hydrochloric acid (40 mL; 2N) and then extracted with dichloromethane (2×30 mL). The organic phases were combined, washed successively with water and aqueous saturated sodium bicarbonate, dried over magnesium sulfate, filtered, and concentrated under reduced pressure. The crude residue was purified by chromatography on silica gel eluting with ethyl acetate/hexanes (1:6). Recrystallization from hexanes/ethyl acetate gave the title product as white crystals (2.54 g).

### II. 1-Bromo-2,4-dimethyl-3-nitrobenzene (2)

To a solution of 1,3-dimethyl-2-nitrobenzene (5.42 mL; 40.0 mmol) in trifluoroacetic acid (40 mL) was added N-bromosuccinimide (15.6 g; 88.0 mmol) and iron (66 mg; 1.2 mmol). The reaction mixture was heated at 75° C. for 3 days, allowed to cool to room temperature and then the solvent was removed under reduced pressure. The resulting residue was dissolved in ethyl acetate and washed with aqueous saturated sodium bicarbonate, dried over magnesium sulfate, filtered, and concentrated under reduced pressure. The crude residue was purified by chromatography on silica gel to yield the title product as a white solid (6.42 g).

### III. 3-Bromo-2,6-dimethylaniline (3)

To a solution of 2 (1.01 g; 4.39 mmol) in acetic acid (18 mL) was added iron (0.98 g; 17.56 mmol). The reaction mixture was heated at 80° C. open to room atmosphere for 1 day, allowed to cool to room temperature and filtered to remove solids that had formed. The filtrate was basified by addition on aqueous sodium hydroxide (2N) and then extracted with ethyl acetate (4×30 mL). The organic layers were combined, dried over magnesium sulfate, filtered, and concentrated under reduced pressure to give the title compound as a yellow oil (0.75 g).

### IV. 2,6-Dimethyl-3-(pyrrolidin-1-yl)aniline (4)

To a solution of 3 (0.75 g; 3.75 mmol) in toluene (10 mL) were added pyrrolidine (0.38 mL; 4.5 mmol) and sodium tert-butoxide (0.72 g; 7.5 mmol) sequentially. The reaction mixture was deoxygenated by placing under vacuum and then purging with a dry nitrogen atmosphere (3 times), stirred at room temperature for 30 minutes and then bis(dibenzylideneacetone)palladium(0) (34 mg; 0.037 mmol) and (±)-BINAP (93 mg; 0.15 mmol) were added sequentially. The reaction mixture was deoxygenated as before (3 times) and then heated at 100° C. for 16 hours under a nitrogen atmosphere. After cooling to room temperature, the reaction mixture was quenched with water (15 mL) and extracted with ethyl acetate (2×15 mL). The aqueous layer was separated and basified to pH 9 by addition of aqueous sodium hydroxide (2N) and then extracted with ethyl acetate (2×15 mL). All ethyl acetate layers were combined, dried over magnesium sulfate, filtered, and concentrated under reduced pressure. The resulting crude product was purified by column chromatography on silica gel, eluting with ethyl acetate/hexanes (1:8) to give the title compound as a yellow oil (0.49 g).

### V. 4-Fluoro-2,6-dimethyl-3-(pyrrolidin-1-yl)aniline (5)

To a cooled (0° C.) solution of 4 (0.49 g; 2.57 mmol) in dichloroethane (3.9 mL) was added 1-fluoro-2,4,6-trimethylpyridinium trifluoromethanesulfonate (1.12 g; 3.86 mmol) in small portions. After stirring at room temperature overnight, the reaction mixture was diluted with ethyl acetate/hexanes (1:10) and filtered through a short silica gel column. The filtrate was concentrated under reduced pressure and the resulting residue was purified by column chromatography on silica gel, eluting with an ethyl acetate/hexanes gradient (0% 10%) to give the title product as a light brown oil (0.14 g).

### VI. N-[4-Fluoro-2,6-dimethyl-3-(pyrrolidin-1-yl)phenyl]-3-(4-fluorophenylsulfonamido)thiophene-2-carboxamide (6)

To a cooled (0° C.) solution of 5 (104 mg; 0.50 mmol) in toluene (4 mL) was added trimethylaluminum (0.83 mL; 1.67 mmol; 2M) slowly via syringe. After stirring the reaction mixture at 0° C. for 30 minutes and then at room temperature for 1 hour, 1 (131 mg; 0.42 mmol) was added in one portion. Stirring was continued at room temperature for 2 hours and then at 100° C. overnight. The reaction mixture was cooled to room temperature, quenched with aqueous hydrochloric acid (10 mL; 2N) and stirred for an additional 20 minutes at room temperature. Aqueous saturated sodium bicarbonate was added to the reaction mixture to adjust the pH to 4 and then extracted with ethyl acetate (2×50 mL). The organic layers were combined, dried over magnesium sulfate, filtered, and concentrated under reduced pressure. The resulting crude product was purified by column chromatography on silica gel, eluting with ethyl acetate/hexanes (1:7) followed by recrystallization from chloroform/hexanes to give the title compound as a yellow solid (101 mg).

### VII. N-[4-Fluoro-2,6-dimethyl-3-(pyrrolidin-1-yl)phenyl]-3-(4-fluorophenylsulfonamido)thiophene-2-carboxamide, Sodium Salt (7)

To a solution of 6 (99.0 mg; 0.2 mmol) in methanol (3 mL) was added aqueous sodium hydroxide (2.0 mL; 0.20 mmol; 0.1004N). The reaction mixture was frozen in a dry ice/acetone bath and lyophilized to yield the title compound as a yellow solid (85.0 mg).

Mass Spectral Data (m/z): Measured (M+H)⁺=492.11; Calculated (M+H)⁺=492.12.

### Example 2

### Preparation of N-[4-chloro-2,6-dimethyl-3-(pyrrolidin-1-yl)phenyl]-3-(4-fluorophenylsulfonamido)thiophene-2-carboxamide, Sodium Salt (14)

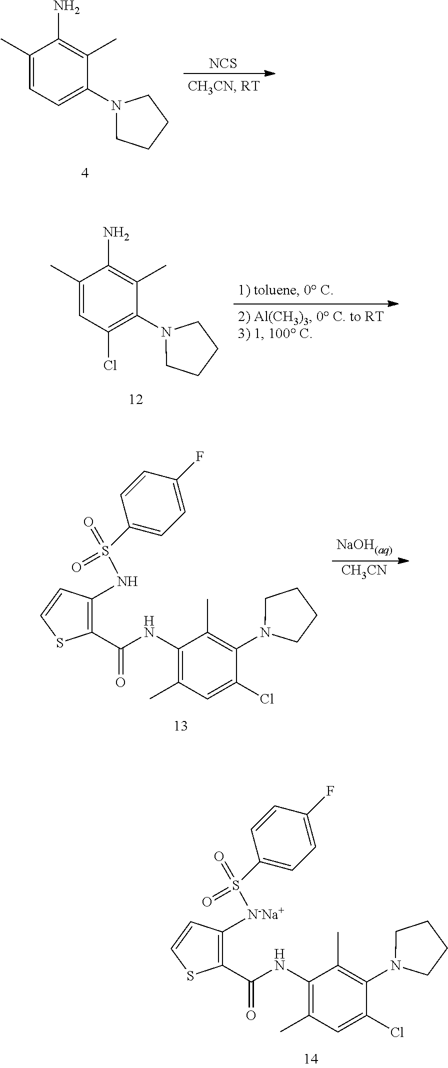

### I. 4-Chloro-2,6-dimethyl-3-(pyrrolidin-1-yl)aniline (12)

To a solution of 4 (1.0 g; 5.26 mmol) in acetonitrile (25 mL) was added N-chlorosuccinimide (0.70 g; 5.26 mmol). The reaction mixture was stirred at room temperature overnight and then the solvent was removed under reduced pressure. The resulting residue was dissolved in ethyl acetate and washed with water and saturated aqueous sodium chloride, dried over magnesium sulfate, filtered, and concentrated under reduced pressure. The crude residue was purified by column chromatography on silica gel, eluting with ethyl acetate/hexanes (1:3) to yield the title product as a light brown oil (1.0 g).

### II. N-[4-Chloro-2,6-dimethyl-3-(pyrrolidin-1-yl)phenyl]-3-(4-fluorophenylsulfonamido)thiophene-2-carboxamide (13)

To a cooled (0° C.) solution of 12 (0.13 g; 0.58 mmol l) in toluene (4 mL) was added trimethylaluminum (0.58 mL; 1.16 mmol; 2M in hexanes) slowly via syringe. After stirring the reaction mixture at 0° C. for 10 minutes and then at room temperature for 30 minutes, 1 (0.12 g; 0.39 mmol) was added in one portion. Stirring was continued at room temperature for 2 hours and then the reaction mixture was heated at 110° C. overnight. The reaction mixture was cooled to room temperature, quenched with aqueous hydrochloric acid (20 mL; 2N), and then diluted with ethyl acetate. The organic layer was separated and washed with water, dried over magnesium sulfate, filtered, and concentrated under reduced pressure. The resulting crude product was purified by column chromatography on silica gel, eluting with ethyl acetate/hexanes (1:5) to give the title compound was obtained as an off-white solid (78 mg).

### III. N-[4-Chloro-2,6-dimethyl-3-(pyrrolidin-1-yl)phenyl]-3-(4-fluorophenylsulfonamido)thiophene-2-carboxamide, Sodium Salt (14)

To a solution of 13 (78 mg; 0.154 mmol) in acetonitrile (2 mL) was added aqueous sodium hydroxide (1.54 mL; 0.155 mmol; 0.1004N) and water (2 mL). The acetonitrile was removed under reduced pressure and then reaction mixture was frozen in a dry ice/acetone bath and lyophilized to yield the title compound as a pale yellow solid (80 mg).

Mass Spectral Data (m/z): Measured (M+H)⁺=508.15; Calculated (M+H)⁺=508.09.

### Example 3

### Preparation of N-[6-chloro-2,4-dimethyl-3-(pyrrolidin-1-yl)phenyl]-3-(4-fluorophenylsulfonamido)thiophene-2-carboxamide, Sodium Salt (20)

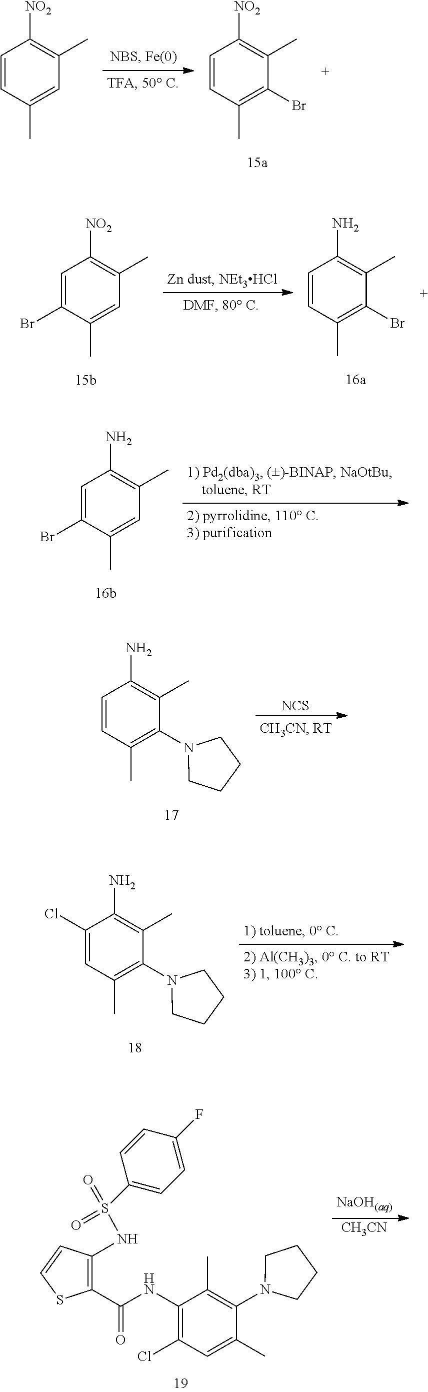

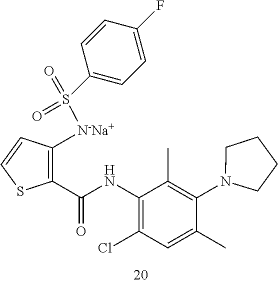

### I. 2-Bromo-1,3-dimethyl-4-nitrobenzene (15a)

Synthesized as described for 2 using 2,4-dimethyl-1-nitrobenzene (5.05 g; 33.4 mmol), N-bromosuccinimide (14.7 g; 82.6 mmol), iron (powder) (56 mg; 1.0 mmol) and trifluoroacetic acid (35 mL) and the reaction was heated at 50° C. Purification by column chromatography on silica gel, eluting with ethyl acetate/hexanes gave a 1:1 mixture of the title compound (15a) and 1-bromo-2,4-dimethyl-5-nitrobenzene (15b) as a white semi-solid (4.57 g).

### II. 3-Bromo-2,4-dimethylaniline (16a)

A suspension of 15a/15b (1:1 mixture of isomers; 4.57 g; 19.9 mmol), zinc dust (5.84 g; 89.4 mmol) and triethylamine hydrochloride (15.04 g; 109 mmol) in dimethylformamide (65 mL) was heated at 80° C. for 4 hours. The reaction mixture was allowed to cool to room temperature and filtered through Celite®. The filtrate was washed with aqueous sodium hydroxide (1N), dried over magnesium sulfate, filtered, and concentrated under reduced pressure. The crude residue was purified by column chromatography on silica gel, eluting with ethyl acetate/hexanes (1:3) to give a mixture of the title compound (16a) and 5-bromo-2,4-dimethylaniline (16b) as a yellow solid (3.27 g).

### III. 2,4-Dimethyl-3-(pyrrolidin-1-yl)aniline (17)

A suspension of 16a/16b (3.27 g; 16.3 mmol), sodium tert-butoxide (3.24 g; 33.7 mmol), bis(dibenzylideneacetone)palladium(0) (0.45 g; 0.49 mmol) and (±)-BINAP (0.94 g; 1.50 mmol) in toluene (40 mL) was deoxygenated by placing under vacuum and then purging with a dry nitrogen atmosphere (3 times). Pyrrolidine (2.07 mL; 24.5 mmol) was added, the reaction mixture was deoxygenated as before (3 times) and then heated at 110° C. for 24 hours under a nitrogen atmosphere. After cooling to room temperature, the reaction mixture was diluted with ethyl acetate, washed with water, dried over magnesium sulfate, filtered, and concentrated under reduced pressure. The resulting crude product was purified by column chromatography on silica gel, eluting with ethyl acetate/hexanes to give the title compound as a brown oil (0.55 g).

### IV. 6-Chloro-2,4-dimethyl-3-(pyrrolidin-1-yl)aniline (18)

Synthesized as described for 12 using 17 (0.55 g; 2.89 mmol), N-chlorosuccinimide (0.39 g; 2.89 mmol) and acetonitrile (15 mL). This afforded the title compound as a brown oil (0.185 g).

### V. N-[6-Chloro-2,4-dimethyl-3-(pyrrolidin-1-yl)phenyl]-3-(4-fluorophenylsulfonamido)thiophene-2-carboxamide (19)

Synthesized as described for 13 using 18 (88 mg; 0.39 mmol), 1 (103 mg; 0.33 mmol), trimethylaluminum (0.65 mL; 1.30 mmol; 2M in hexanes) and toluene (4 mL). Purification by column chromatography on silica gel, eluting with ethyl acetate/hexanes (1:8) followed by recrystallization from ethyl acetate/hexanes yielded the title compound as an off-white solid (102 mg).

### VI. N-[6-Chloro-2,4-dimethyl-3-(pyrrolidin-1-yl)phenyl]-3-(4-fluorophenylsulfonamido)thiophene-2-carboxamide, Sodium Salt (20)

Synthesized as described for 11 using 19 (102 mg; 0.20 mmol), aqueous sodium hydroxide (2.0 mL; 0.20 mmol; 0.1004N), acetonitrile (4 mL) and water (4 mL). This afforded the title compound a pale yellow solid (102 mg).

Mass Spectral Data (m/z): Measured (M+H)⁺=508.10; Calculated (M+H)⁺=508.09.

### Example 4

### Preparation of N-[6-chloro-2,4-dimethyl-3-(pyrrolidin-1-yl)phenyl]-3-(phenylsulfonamido)thiophene-2-carboxamide, Sodium Salt (23)

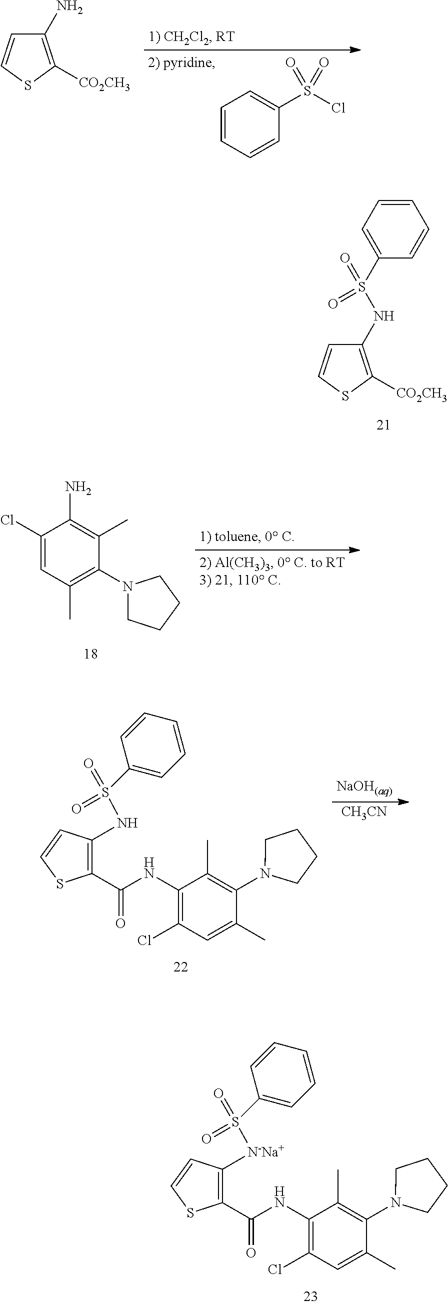

### I. Methyl 3-(phenylsulfonamido)thiophene-2-carboxylate (21)

Synthesized as described for 1 using methyl 3-aminothiophene-2-carboxylate (2.0 g; 12.7 mmol), benzenesulfonyl chloride (2.36 g; 13.4 mmol), pyridine (4 mL) and dichloromethane (15 mL). This afforded the title compound a pale pink solid (3.55 g).

### II. N-[6-Chloro-2,4-dimethyl-3-(pyrrolidin-1-yl)phenyl]-3-(phenylsulfonamido)thiophene-2-carboxamide (22)

Synthesized as described for 13 using 18 (101 mg; 0.45 mmol), 21 (111 mg; 0.37 mmol), trimethylaluminum (0.75 mL; 1.50 mmol; 2M in hexanes) and toluene (4 mL). Purification by column chromatography on silica gel, eluting with ethyl acetate/hexanes (1:8) followed by recrystallization from ethyl acetate/hexanes yielded the title compound as a pale yellow solid (93 mg).

### III. N-[6-Chloro-2,4-dimethyl-3-(pyrrolidin-1-yl)phenyl]-3-(phenylsulfonamido)thiophene-2-carboxamide, Sodium Salt (23)

To a solution of 22 (91 mg; 0.186 mmol) in acetonitrile (3 mL) was added aqueous sodium hydroxide (1.85 mL; 0.186 mmol; 0.1004N) and water (2 mL). The acetonitrile was removed under reduced pressure and then the reaction mixture was filtered. The filtrate was frozen in a dry ice/acetone bath and lyophilized to yield the title compound as a pale yellow solid (98 mg).

Mass Spectral Data (m/z): Measured (M+H)⁺=490.14; Calculated (M+H)⁺=490.10.

### Example 5

### Preparation of N-[4-chloro-2,6-dimethyl-3-(morpholin-4-yl)phenyl]-3-(4-fluorophenylsulfonamido)thiophene-2-carboxamide, Sodium Salt (28)

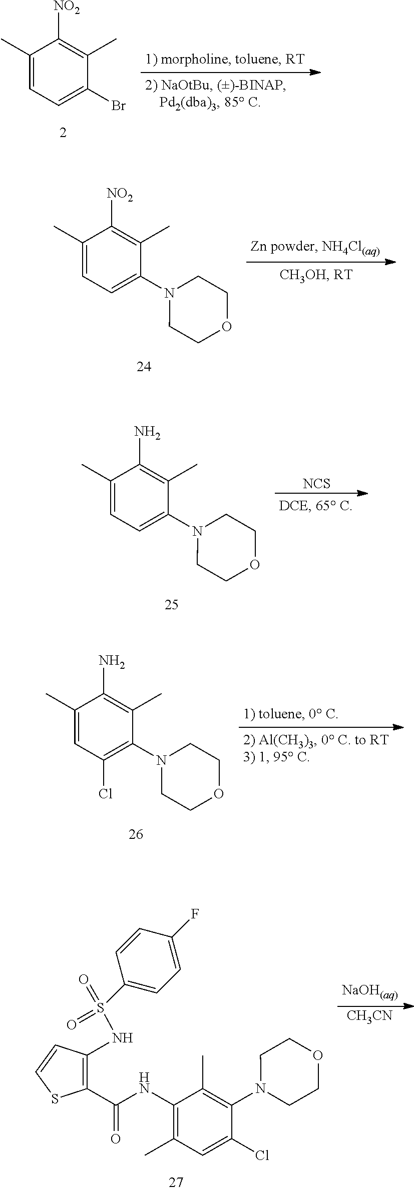

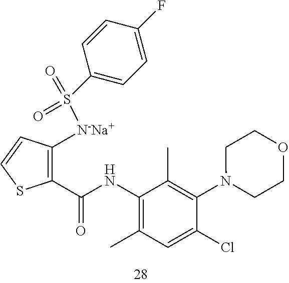

### I. 4-(2,4-Dimethyl-3-nitrophenyl)morpholine (24)

To a solution of 2 (7.57 g; 32.9 mmol) and morpholine (3.50 mL; 40.2 mmol) in toluene (100 mL) at room temperature under a dry nitrogen atmosphere were added sodium tert-butoxide (6.38 g; 66.4 mmol), (±)-BINAP (686 mg; 1.10 mmol) and bis(dibenzylideneacetone)palladium(0) (557 mg; 0.61 mmol) sequentially. The reaction mixture was heated at 85° C. overnight under a nitrogen atmosphere, cooled to room temperature, and then washed with water (100 mL). The aqueous layer was separated and extracted with ethyl acetate (2×30 mL). All ethyl acetate layers were combined, filtered through silica gel, and then concentrated under reduced pressure to give the title compound as a brown viscous oil (4.62 g).

### II. 2,6-Dimethyl-3-(morpholin-4-yl)aniline (25)

Synthesized as described for 16 using 24 (4.62 g; 19.6 mmol), zinc powder (6.45 g; 98.7 mmol), saturated aqueous ammonium chloride (40 mL) and methanol (120 mL) and the reaction mixture was stirred at room temperature overnight. This afforded the title compound as a pale orange solid (2.89 g).

### III. 4-Chloro-2,6-dimethyl-3-(morpholin-4-yl)aniline (26)

Synthesized as described for 12 using 25 (465 mg; 2.25 mmol), N-chlorosuccinimide (440 mg; 3.29 mmol) and 1,2-dichloroethane (10 mL) and the reaction mixture was heated at 65° C. for 3 days. This afforded the title compound as an off-white solid (326 mg).

### IV. N-[4-Chloro-2,6-dimethyl-3-(morpholin-4-yl)phenyl]-3-(4-fluorophenylsulfonamido)thiophene-2-carboxamide (27)

Synthesized as described for 13 using 26 (118 mg; 0.49 mmol), 1 (156 mg; 0.49 mmol), trimethylaluminum (1.0 mL; 2.0 mmol; 2M in hexanes) and toluene (3 mL) and the reaction mixture was heated at 95° C. This afforded the title compound as a white powder (217 mg).

### V. N-[4-Chloro-2,6-dimethyl-3-(morpholin-4-yl)phenyl]-3-(4-fluorophenylsulfonamido)thiophene-2-carboxamide, Sodium Salt (28)

To a flask containing 27 (213 mg; 0.41 mmol) was added acetonitrile (3 mL) and aqueous sodium hydroxide (3.9 mL; 0.41 mmol; 0.105N). The reaction mixture was sonicated and gently heated to dissolve all solids. The resulting solution was allowed to cool to room temperature and then frozen in a dry ice/acetone bath and lyophilized to yield the title compound as a white solid (220 mg).

Mass Spectral Data (m/z): Measured (M−H)⁻=522.12; Calculated (M−H)⁻=522.07.

### Example 6

### Preparation of N-[4-fluoro-2,6-dimethyl-3-(morpholin-4-yl)phenyl]-3-(4-fluorophenylsulfonamido)thiophene-2-carboxamide, Sodium Salt (31)

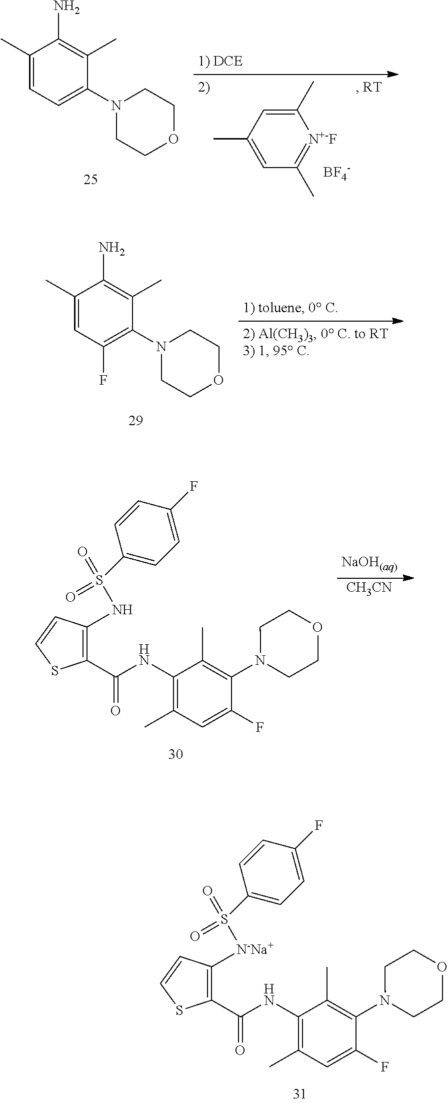

### I. 4-Fluoro-2,6-dimethyl-3-(morpholin-4-yl)aniline (29)

To a solution of 25 (889 mg; 4.30 mmol) in 1,2-dichloroethane (15 mL) was added 1-fluoro-2,4,6-trimethylpyridinium tetrafluoroborate (1.44 g; 6.33 mmol). After stirring at room temperature for 2 days, the reaction mixture was poured into aqueous saturated sodium bicarbonate (50 mL) and extracted with dichloromethane (3×15 mL). The organic layers were combined, dried over magnesium sulfate, filtered, and concentrated under reduced pressure. The resulting crude product was purified by column chromatography on silica gel, eluting with an ethyl acetate/hexanes gradient (15% to 25%) to give the title compound as a brown solid (291 mg).

### II. N-[4-Fluoro-2,6-dimethyl-3-(morpholin-4-yl)phenyl]-3-(4-fluorophenylsulfonamido)thiophene-2-carboxamide (30)

Synthesized as described for 13 using 29 (144 mg; 0.64 mmol), toluene (5 mL), a solution of 1 (209 mg; 0.66 mmol) in toluene (4 mL) and trimethylaluminum (1.5 mL; 3.0 mmol; 2M in hexanes) and the reaction mixture was heated at 95° C. This afforded the title compound as a yellow solid (48 mg).

### III. N-[4-Fluoro-2,6-dimethyl-3-(morpholin-4-yl)phenyl]-3-(4-fluorophenylsulfonamido)thiophene-2-carboxamide, Sodium Salt (31)

Synthesized as described for 28 using 30 (44 mg; 0.09 mmol), aqueous sodium hydroxide (0.9 mL; 0.09 mmol; 0.105N) and acetonitrile (1 mL). This afforded the title compound as a yellow solid (47 mg).

Mass Spectral Data (m/z): Measured (M−H)⁻=506.16; Calculated (M−H)⁻=506.10.

### Example 7

### Preparation of N-[4-chloro-2,6-dimethyl-3-(pyrrolidin-1-yl)phenyl]-3-(phenylsulfonamido)thiophene-2-carboxamide (34)

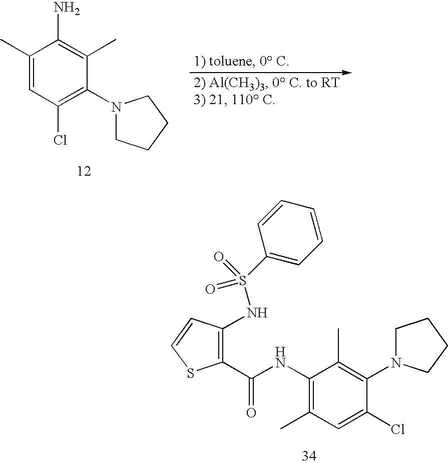

### I. N-[4-Chloro-2,6-dimethyl-3-(pyrrolidin-1-yl)phenyl]-3-(phenylsulfonamido)thiophene-2-carboxamide (34)

Synthesized as described for 13 using 12 (45 mg; 0.2 mmol), 21 (53 mg; 0.18 mmol), trimethylaluminum (0.3 mL; 0.6 mmol; 2M in hexanes) and toluene (1 mL). This afforded the title compound as a tan solid (17 mg).

Mass Spectral Data (m/z): Measured (M+H)⁺=490.16; Calculated (M+H)⁺=490.10.

### Example 8

### Preparation of 3-(4-chlorophenylsulfonamido)-N-[2,4,6-trimethyl-3-(morpholin-4-yl)phenyl]thiophene-2-carboxamide, Sodium Salt (38)

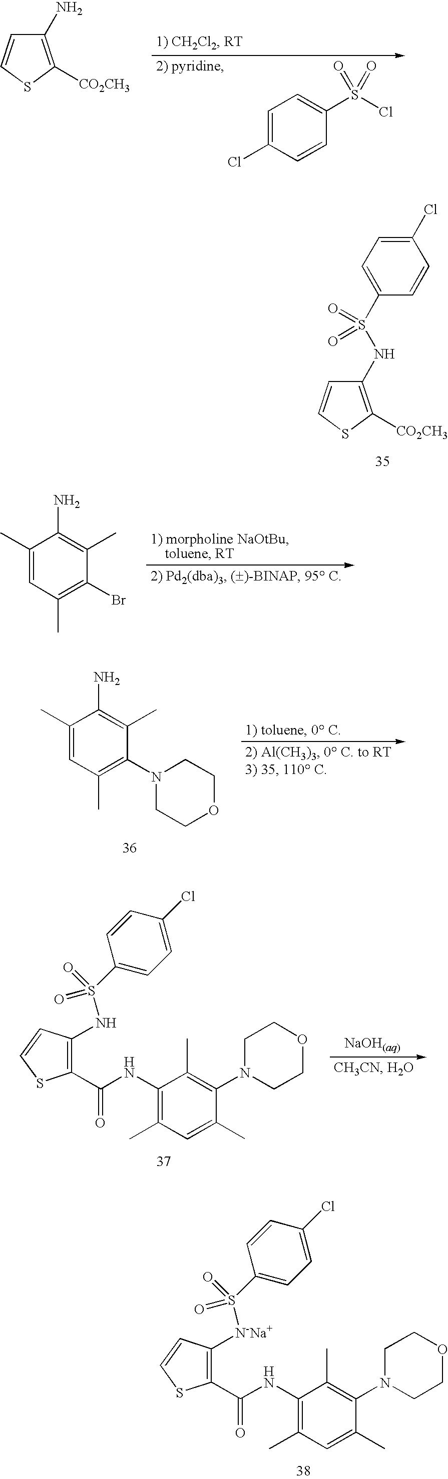

### I. Methyl 3-(4-chlorophenylsulfonamido)thiophene-2-carboxylate (35)

Synthesized as described for 1 using methyl 3-aminothiophene-2-carboxylate (745 mg; 4.7 mmol), 4-chlorobenzene-1-sulfonyl chloride (1.20 g; 5.7 mmol), pyridine (0.8 mL) and dichloromethane (12 mL). This afforded the title compound as a yellow solid (901 mg).

### II. 2,4,6-Trimethyl-3-(morpholin-4-yl)aniline (36)

Synthesized as described for 4 using 3-bromo-2,4,6-trimethylaniline (10.0 g; 46.7 mmol), morpholine (5 mL; 57.4 mmol), sodium tert-butoxide (9.0 g; 93.6 mmol), (±)-BINAP (1.16 g; 1.86 mmol) and bis(dibenzylideneacetone)palladium(0) (0.85 g; 0.93 mmol) and toluene (130 mL) and the reaction mixture was heated at 95° C. This afforded the title compound as pale orange solid (2.01 g).

### III. 3-(4-Chlorophenylsulfonamido)-N-[2,4,6-trimethyl-3-(morpholin-4-yl)phenyl]thiophene-2-carboxamide (37)

Synthesized as described for 13 using 36 (111 mg; 0.55 mmol), 35 (140 mg; 0.42 mmol), trimethylaluminum (0.63 mL; 1.26 mmol; 2M in hexanes) and toluene (4.2 mL). This afforded the title compound as a pale pink solid (73 mg).

### IV. 3-(4-Chlorophenylsulfonamido)-N-[2,4,6-trimethyl-3-(morpholin-4-yl)phenyl]thiophene-2-carboxamide, Sodium Salt (38)

To a flask containing 37 (70 mg; 0.13 mmol) was added acetonitrile followed by aqueous sodium hydroxide (1.3 mL; 0.13 mmol; 0.1N) and water and then heated under reduced pressure for 5 minutes. The reaction mixture was frozen in a dry ice/acetone bath and lyophilized to yield the title compound as a white solid (71 mg).

Mass Spectral Data (m/z): Measured (M−H)⁻=518.14; Calculated (M−H)⁻=518.10.

### Example 9

### Preparation of N-[4-chloro-2,6-dimethyl-3-(morpholin-4-yl)phenyl]-3-(4-chlorophenylsulfonamido)thiophene-2-carboxamide, Sodium Salt (40)

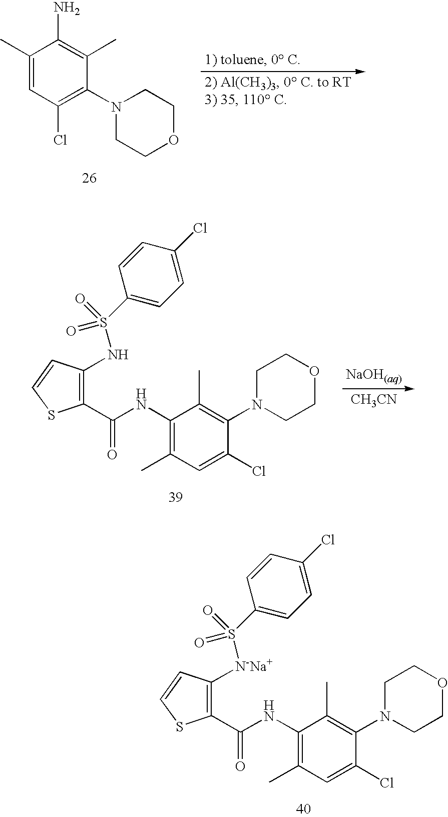

### I. N-[4-Chloro-2,6-dimethyl-3-(morpholin-4-yl)phenyl]-3-(4-chlorophenylsulfonamido)thiophene-2-carboxamide (39)

Synthesized as described for 13 using 26 (159 mg; 0.66 mmol), 35 (145 mg; 0.44 mmol), trimethylaluminum (0.7 mL; 1.4 mmol; 2M in hexanes) and toluene (4.4 mL). This afforded the title compound as a white solid (200 mg).

### II. N-[4-Chloro-2,6-dimethyl-3-(morpholin-4-yl)phenyl]-3-(4-chlorophenylsulfonamido)thiophene-2-carboxamide, Sodium Salt (40)

To a solution of 39 (200 mg; 0.37 mmol) in acetonitrile was added aqueous sodium hydroxide (3.7 mL; 0.37 mmol; 0.1N) and water (20 mL). The reaction mixture was frozen in a dry ice/acetone bath and lyophilized to yield the title compound as a white solid (208 mg).

Mass Spectral Data (m/z): Measured (M−H)⁻=538.09; Calculated (M−H)⁻=538.04.

### Example 10

### Preparation of 3-(2-fluoro-4-methylphenylsulfonamido)-N-[2,4,6-trimethyl-3-(morpholin-4-yl)phenyl]thiophene-2-carboxamide, Sodium Salt (43)

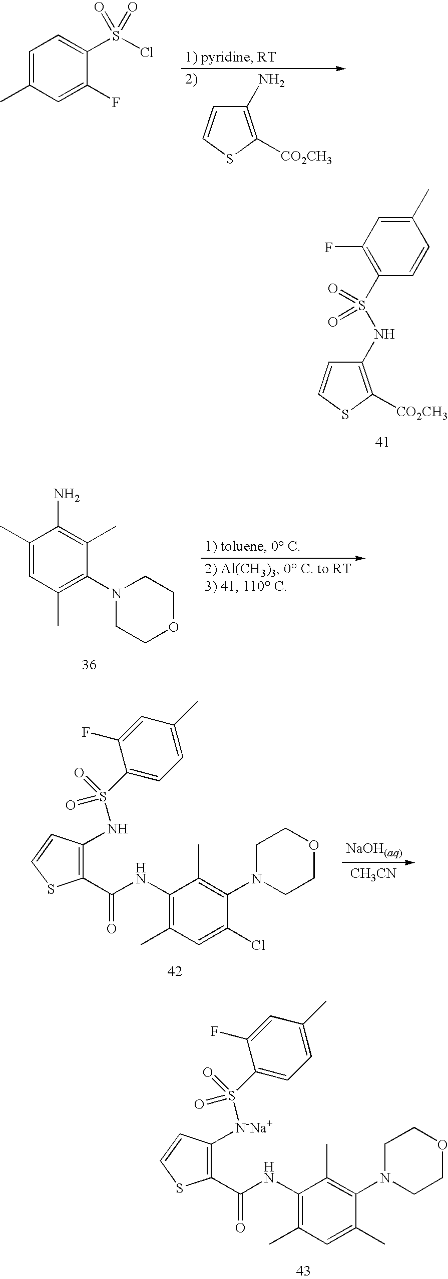

### I. Methyl 3-(2-fluoro-4-methylphenylsulfonamido)thiophene-2-carboxylate (41)

To a solution of 2-fluoro-4-methylbenzene-1-sulfonyl chloride (564 mg; 2.7 mmol) in pyridine (2.5 mL) was added methyl 3-aminothiophene-2-carboxylate (350 mg; 2.2 mmol). After stirring at room temperature overnight, the reaction mixture was diluted with water (5 mL). The resulting precipitate was collected by filtration to give the title compound as a pale yellow solid (695 mg).

### II. 3-(2-Fluoro-4-methylphenylsulfonamido)-N-[2,4,6-trimethyl-3-(morpholin-4-yl)phenyl]thiophene-2-carboxamide (42)

Synthesized as described for 13 using 36 (289 mg; 1.3 mmol), 41 (330 mg; 1.0 mmol), trimethylaluminum (1.5 mL; 3.0 mmol; 2M in hexanes) and toluene (7 mL). This afforded the title compound as a red solid (324 mg).

### III. 3-(2-Fluoro-4-methylphenylsulfonamido)-N-[2,4,6-trimethyl-3-(morpholin-4-yl)phenyl]thiophene-2-carboxamide, Sodium Salt (43)

Synthesized as described for 38 using 42 (322 mg; 0.62 mmol), aqueous sodium hydroxide (6.2 mL; 0.62 mmol; 0.1N) and acetonitrile. This afforded the title compound as a pale pink solid (258 mg).

Mass Spectral Data (m/z): Measured (M+H)⁺=518.18; Calculated (M+H)⁺=518.16.

### Example 11

### Preparation of 3-(3-fluoro-2-methylphenylsulfonamido)-N-[2,4,6-trimethyl-3-(morpholin-4-yl)phenyl]thiophene-2-carboxamide, Sodium Salt (46)

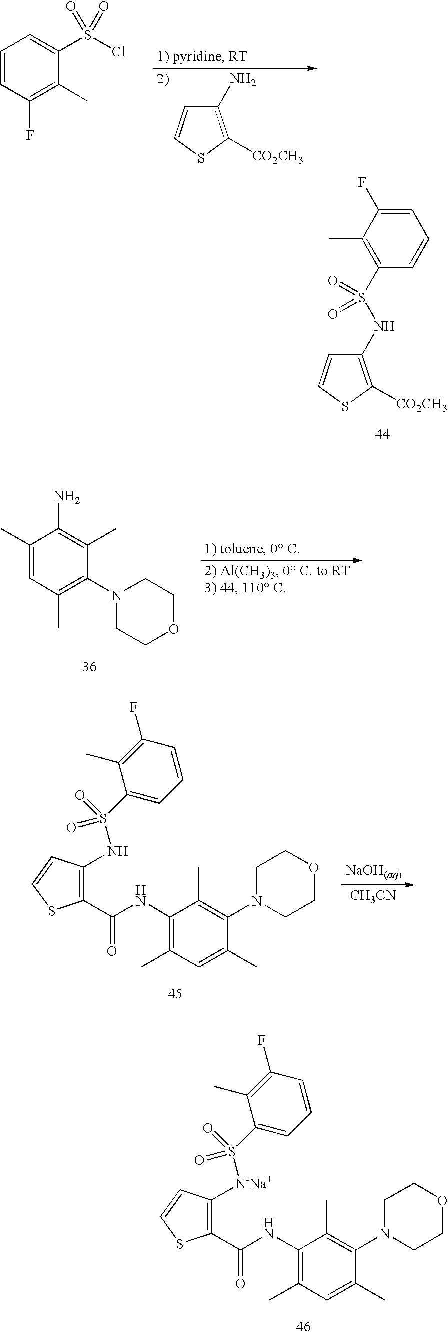

### I. Methyl 3-(3-fluoro-2-methylphenylsulfonamido)thiophene-2-carboxylate (44)

Synthesized as described for 41 using methyl 3-aminothiophene-2-carboxylate (350 mg; 2.2 mmol), 3-fluoro-2-methylbenzene-1-sulfonyl chloride (564 mg; 2.7 mmol), and pyridine (2.5 mL). This afforded the title compound as a pale yellow solid (450 mg).

### II. 3-(3-Fluoro-2-methylphenylsulfonamido)-N-[2,4,6-trimethyl-3-(morpholin-4-yl)phenyl]thiophene-2-carboxamide (45)

Synthesized as described for 13 using 36 (289 mg; 1.3 mmol), 44 (330 mg; 1.0 mmol), trimethylaluminum (1.5 mL; 3.0 mmol; 2M in hexanes) and toluene (7 mL). This afforded the title compound as a reddish-brown solid (366 mg).

### III. 3-(3-Fluoro-2-methylphenylsulfonamido)-N-[2,4,6-trimethyl-3-(morpholin-4-yl)phenyl]thiophene-2-carboxamide, Sodium Salt (46)

Synthesized as described for 38 using 45 (364 mg; 0.70 mmol), aqueous sodium hydroxide (7.0 mL; 0.70 mmol; 0.1N) and acetonitrile. This afforded the title compound as a pale pink solid (242 mg).

Mass Spectral Data (m/z): Measured (M+H)⁺=518.18; Calculated (M+H)⁺=518.16.

### Example 12

### Preparation of 3-(2-methoxyphenylsulfonamido)-N-[2,4,6-trimethyl-3-(morpholin-4-yl)phenyl]thiophene-2-carboxamide, Sodium Salt (49)

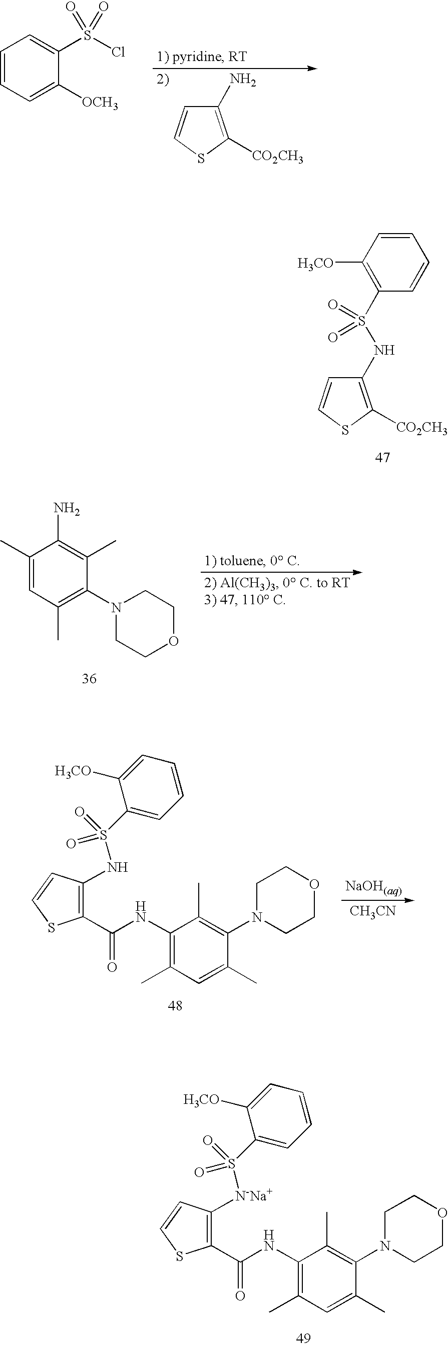

### I. Methyl 3-(2-methoxyphenylsulfonamido)thiophene-2-carboxylate (47)

Synthesized as described for 41 using methyl 3-aminothiophene-2-carboxylate (350 mg; 2.2 mmol), 2-methoxybenzene-1-sulfonyl chloride (559 mg; 2.7 mmol), and pyridine (2.5 mL). This afforded the title compound as an off-white solid (668 mg).

### II. 3-(2-Methoxyphenylsulfonamido)-N-[2,4,6-trimethyl-3-(morpholin-4-yl)phenyl]thiophene-2-carboxamide (48)

Synthesized as described for 13 using 36 (300 mg; 1.35 mmol), 47 (363 mg; 1.11 mmol), trimethylaluminum (1.7 mL; 3.4 mmol; 2M in hexanes) and toluene (5 mL). This afforded the title compound as an off-white solid (319 mg).

### III. 3-(2-Methoxyphenylsulfonamido)-N-[2,4,6-trimethyl-3-(morpholin-4-yl)phenyl]thiophene-2-carboxamide, Sodium Salt (49)

Synthesized as described for 38 using 48 (318 mg; 0.62 mmol), aqueous sodium hydroxide (6.2 mL; 0.62 mmol; 0.1N) and acetonitrile. This afforded the title compound as a pale pink solid (330 mg).

Mass Spectral Data (m/z): Measured (M+H)⁺=516.18; Calculated (M+H)⁺=516.16.

### Example 13

### Preparation of 3-(4-chloro-2-methylphenylsulfonamido)-N-[2,4,6-trimethyl-3-(morpholin-4-yl)phenyl]thiophene-2-carboxamide, Sodium Salt (53)

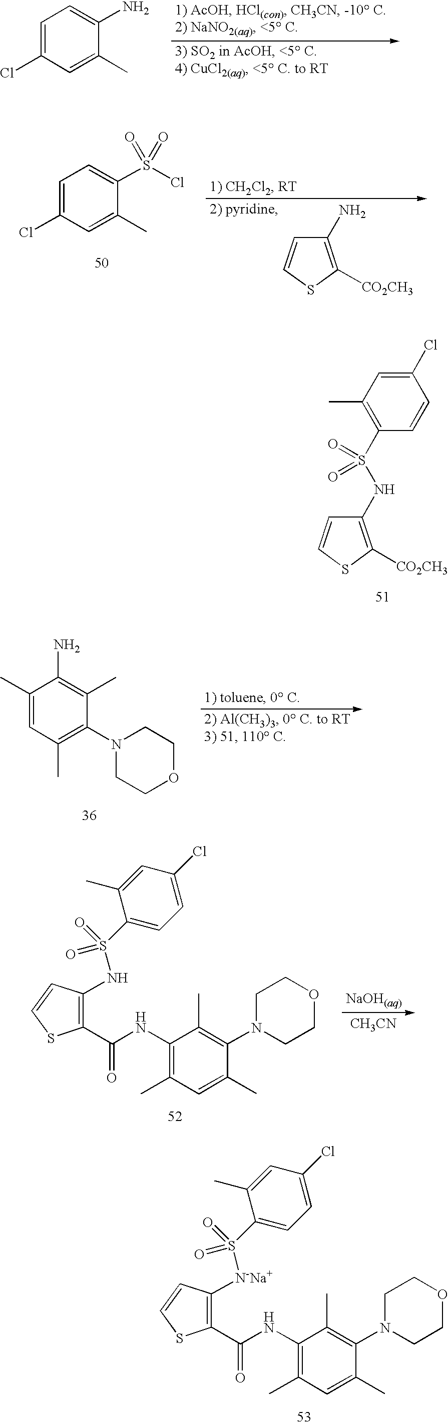

### I. 4-Chloro-2-methylbenzene-1-sulfonyl chloride (50)

To a cooled solution (−10° C.) of 4-chloro-2-methylaniline (5.00 g; 35.3 mmol) in acetonitrile (353 mL) was added acetic acid (20 mL) and concentrated hydrochloric acid (10 mL). To this mixture, a solution of sodium nitrite (2.92 g; 42.4 mmol) dissolved in water (5.5 mL) was added dropwise via syringe at a rate sufficient to keep the reaction temperature below 5° C. After stirring for 10 minutes following this addition, a saturated solution of sulfur dioxide dissolved in glacial acetic acid (125 mL; prepared by passing gaseous sulfur dioxide through a gas dispersion tube into glacial acetic acid for 30 minutes) was added dropwise via an addition funnel. The rate of addition of the sulfur dioxide saturated acetic acid solution was such that the internal reaction temperature did not rise above 5° C. (about 30 minutes). Following addition of the sulfur dioxide solution, a solution of copper(II) chloride dihydrate (5.93 g, 44.1 mmol) in water (5.5 mL) was added to the reaction mixture. The reaction was stirred for 30 minutes at 0° C. then warmed to room temperature and stirred overnight. The reaction mixture was poured into ice water (1 L) and stirred 30 minutes. The resulting precipitate was collected by filtration and then dried under vacuum to give the title product as an off-white solid (5.3 g).

### II. Methyl 3-(4-chloro-2-methylphenylsulfonamido)thiophene-2-carboxylate (51)

Synthesized as described for 41 using methyl 3-aminothiophene-2-carboxylate (350 mg; 2.2 mmol), 50 (605 mg; 2.7 mmol), and pyridine (2.5 mL). This afforded the title compound as off-white solid (744 mg).

### III. 3-(4-Chloro-2-methylphenylsulfonamido)-N-[2,4,6-trimethyl-3-(morpholin-4-yl)phenyl]thiophene-2-carboxamide (52)

Synthesized as described for 13 using 36 (300 mg; 1.35 mmol), 51 (382 mg; 1.10 mmol), trimethylaluminum (1.7 mL; 3.4 mmol; 2M in hexanes) and toluene (5 mL). Recrystallization from acetonitrile/water gave the title compound as an off-white solid (319 mg).

### IV. 3-(4-Chloro-2-methylphenylsulfonamido)-N-[2,4,6-trimethyl-3-(morpholin-4-yl)phenyl]thiophene-2-carboxamide, Sodium Salt (53)

To a flask containing 52 (317 mg; 0.6 mmol) was added acetonitrile (1 mL) and aqueous sodium hydroxide (6 mL; 0.6 mmol; 0.1N). The reaction mixture was sonicated and gently heated to dissolve all solids. The resulting solution was allowed to cool to room temperature and then frozen in a dry ice/acetone bath and lyophilized to yield the title compound as a off-white solid (318 mg).

Mass Spectral Data (m/z): Measured (M+H)⁺=534.13; Calculated (M+H)⁺=534.13.

### Example 14

### Preparation of 3-(2-chloro-4-methylphenylsulfonamido)-N-[2,4,6-trimethyl-3-(morpholin-4-yl)phenyl]thiophene-2-carboxamide, Sodium Salt (57)

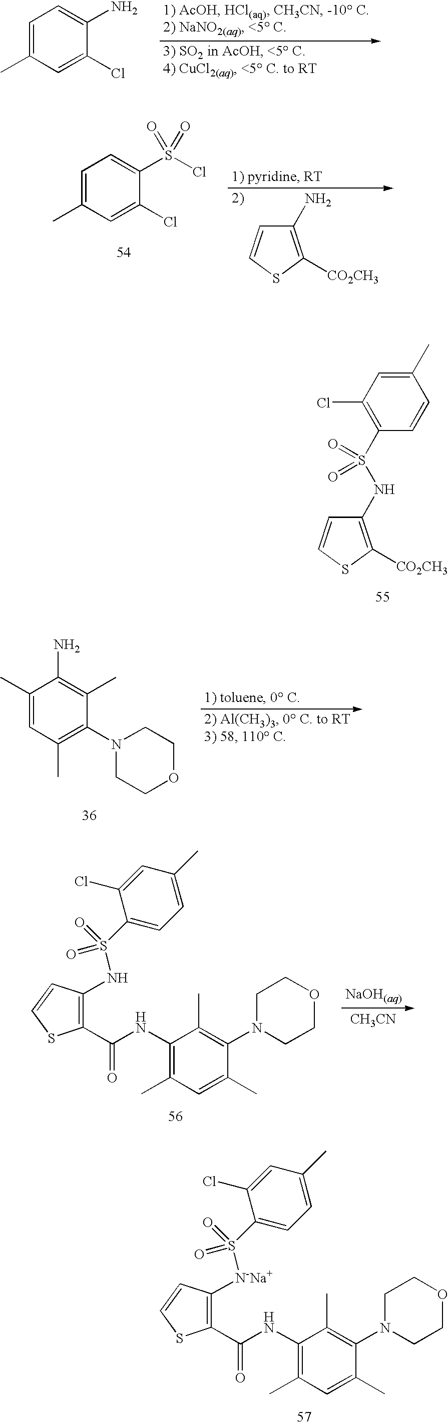

### I. 2-Chloro-4-methylbenzene-1-sulfonyl chloride (54)

Synthesized as described for 50 using 2-chloro-4-methylaniline (5.00 g; 35.3), acetonitrile (353 mL), acetic acid (20 mL), concentrated hydrochloric acid (10 mL), a solution of sodium nitrite (2.92 g; 42.4 mmol) dissolved in water (5.5 mL), a saturated solution of sulfur dioxide in glacial acetic acid (125 mL; prepared as in example 13), and a solution of copper(II) chloride dihydrate (5.93 g, 44.1 mmol) dissolved in water (5.5 mL). The crude reaction mixture was poured into ice water (1L) and stirred for 30 minutes, at which time a dark brown oil had formed at the bottom of the flask. The aqueous layer was decanted and the oil was taken up in dichloromethane, dried over MgSO₄ and filtered. The filtrate was concentrated under reduced pressure to afford the title compound as a dark brown oil (4.2 g).

### II. Methyl 3-(2-chloro-4-methylphenylsulfonamido)thiophene-2-carboxylate (55)

Synthesized as described for 41 using methyl 3-aminothiophene-2-carboxylate (350 mg; 2.2 mmol), 54 (414 mg; 1.85 mmol) and pyridine (2 mL). This afforded the title compound as an off-white solid (690 mg).

### III. 3-(2-Chloro-4-methylphenylsulfonamido)-N-[2,4,6-trimethyl-3-(morpholin-4-yl)phenyl]thiophene-2-carboxamide (56)

Synthesized as described for 13 using 36 (322 mg; 1.4 mmol), 55 (385 mg; 1.1 mmol), trimethylaluminum (1.65 mL; 3.3 mmol; 2M in hexanes) and toluene (5.5 mL). This afforded the title compound as a yellow solid (252 mg).

### IV. 3-(2-Chloro-4-methylphenylsulfonamido)-N-[2,4,6-trimethyl-3-(morpholin-4-yl)phenyl]thiophene-2-carboxamide, Sodium Salt (57)

Synthesized as described for 53 using 56 (252 mg; 0.47 mmol), aqueous sodium hydroxide (4.7 mL; 0.47 mmol; 0.1N) and acetonitrile. This afforded the title compound as an off-white solid (211 mg).

Mass Spectral Data (m/z): Measured (M−H)⁻=532.20; Calculated (M−H)⁻=532.11.

### Example 15

### Preparation of 3-(3-fluoro-4-methylphenylsulfonamido)-N-[2,4,6-trimethyl-3-(morpholin-4-yl)phenyl]thiophene-2-carboxamide, Sodium Salt (60)

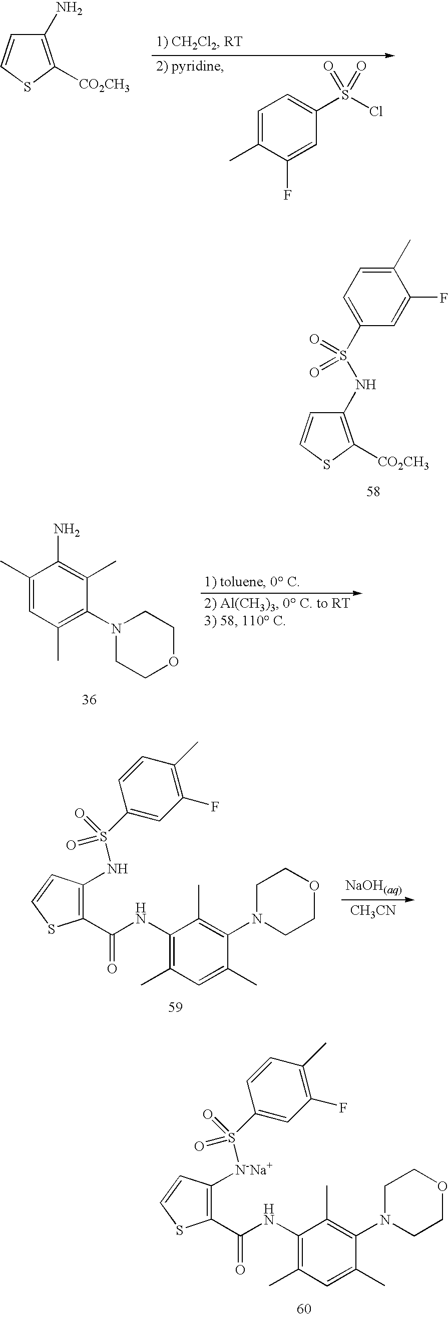

### I. Methyl 3-(3-fluoro-4-methylphenylsulfonamido)thiophene-2-carboxylate (58)

To a solution of 3-fluoro-4-methylbenzene-1-sulfonyl chloride (563 mg; 2.7 mmol) in dichloromethane (5.5 mL) was added pyridine (0.36 mL) and methyl 3-aminothiophene-2-carboxylate (350 mg; 2.2 mmol) sequentially. After stirring at room temperature overnight, the reaction mixture was diluted with water and extracted with ethyl acetate (2×25 mL). The organic layers were combined, washed successively with water and saturated aqueous sodium chloride, dried over MgSO₄ and filtered. The filtrate was concentrated under reduced pressure to give the title compound as a yellow solid (486 mg).

### II. 3-(3-Fluoro-4-methylphenylsulfonamido)-N-[2,4,6-trimethyl-3-(morpholin-4-yl)phenyl]thiophene-2-carboxamide (59)

Synthesized as described for 13 using 36 (57 mg; 0.26 mmol), 58 (65 mg; 0.25 mmol), trimethylaluminum (0.4 mL; 0.8 mmol; 2M in hexanes) and toluene (1 mL). The crude product was purified by automated silica gel column chromatography (Biotage®) eluting with ethyl acetate/hexanes to give the title compound as an off-white solid (54 mg).

### III. 3-(3-Fluoro-4-methylphenylsulfonamido)-N-[2,4,6-trimethyl-3-(morpholin-4-yl)phenyl]thiophene-2-carboxamide, Sodium Salt (60)

Synthesized as described for 53 using 59 (48 mg; 0.094 mmol), aqueous sodium hydroxide (0.94 mL; 0.094 mmol; 0.1N) and acetonitrile. This afforded the title compound as an off-white solid (51 mg).

Mass Spectral Data (m/z): Measured (M−H)⁻=516.14; Calculated (M−H)⁻=516.14.

### Example 16

### Preparation of N-[4-chloro-2,6-dimethyl-3-(morpholin-4-yl)phenyl]-3-(4-methylphenylsulfonamido)thiophene-2-carboxamide, Sodium Salt (64)

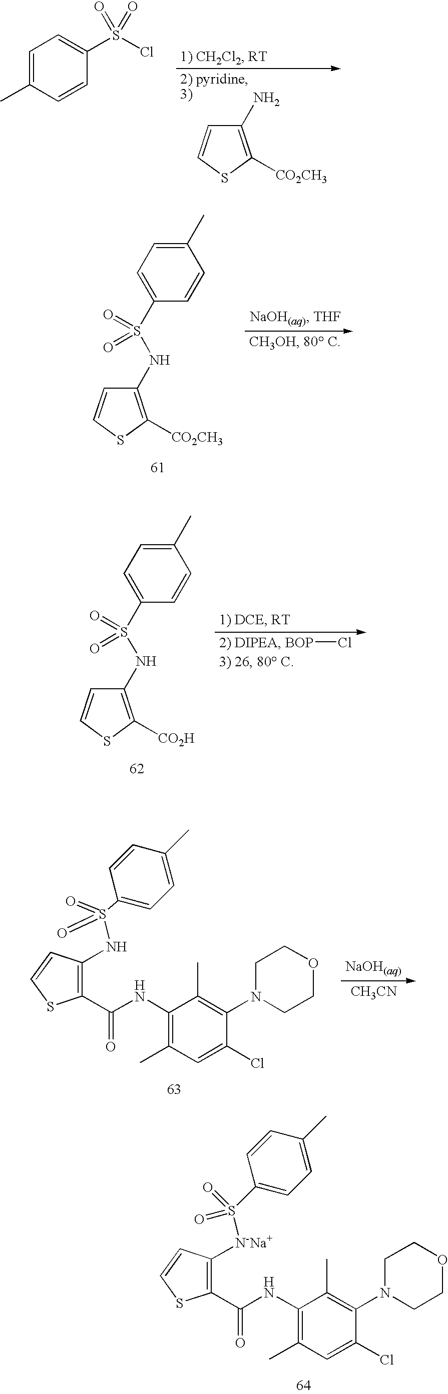

### I. Methyl 3-(4-methylphenylsulfonamido)thiophene-2-carboxylate (61)

Synthesized as described for 58 using methyl 3-aminothiophene-2-carboxylate (2.00 g; 12.8 mmol), 4-methylbenzene-1-sulfonyl chloride (2.92 g; 15.3 mmol), pyridine (2.1 mL) and dichloromethane (32 mL). This afforded the title compound a white solid (2.62 g).

### II. 3-(4-Methylphenylsulfonamido)thiophene-2-carboxylic acid (62)

To a mixture of 61 (1.40 g; 4.5 mmol) in tetrahydrofuran (31 mL) were added aqueous sodium hydroxide (22 mL; 2N) and methanol (11 mL). The reaction mixture was heated at 80° C. overnight, cooled to room temperature and then washed with diethyl ether. The aqueous layer was separated, acidified, and extracted with ethyl acetate (2×20 mL). The ethyl acetate layers were combined, dried over magnesium sulfate, filtered, and concentrated under reduced pressure to give the title compound as a white solid (1.0 g).

### III. N-[4-Chloro-2,6-dimethyl-3-(morpholin-4-yl)phenyl]-3-(4-methylphenylsulfonamido)thiophene-2-carboxamide (63)

To a solution on 62 (178 mg; 0.60 mmol) in 1,2-dichloroethane were added diisopropylethylamine (0.11 mL; 0.63 mmol) and bis(2-oxo-3-oxazolidinyl)phosphinic chloride (115 mg; 0.5 mmol) sequentially. After stirring at room temperature for 20 minutes, a solution of 26 (153 mg; 0.64 mmol) in 1,2-dichloroethane (3.5 mL) was added via syringe. The reaction mixture was heated at 80° C. overnight, cooled to room temperature, quenched by addition of aqueous saturated sodium bicarbonate, and then extracted with ethyl acetate (3×20 mL). The ethyl acetate layers were combined, washed successively with water and aqueous saturated sodium chloride, dried over magnesium sulfate, filtered, and concentrated under reduced pressure. The crude dark solid was purified by automated silica gel column chromatography (Biotage®) eluting with ethyl acetate/hexanes to give the title compound as an off-white solid (69 mg).

### IV. N-[4-Chloro-2,6-dimethyl-3-(morpholin-4-yl)phenyl]-3-(4-methylphenylsulfonamido)thiophene-2-carboxamide, Sodium Salt (64)

Synthesized as described for 38 using 63 (67 mg; 0.13 mmol), aqueous sodium hydroxide (1.3 mL; 0.13 mmol; 0.1N) and acetonitrile. This afforded the title compound as a pale yellow solid (70 mg).

Mass Spectral Data (m/z): Measured (M−H)⁻=518.09; Calculated (M−H)⁻=518.10.

### Example 17

### Preparation of 3-(2-fluorophenylsulfonamido)-N-[2,4,6-trimethyl-3-(morpholin-4-yl)phenyl]thiophene-2-carboxamide, Sodium Salt (67)

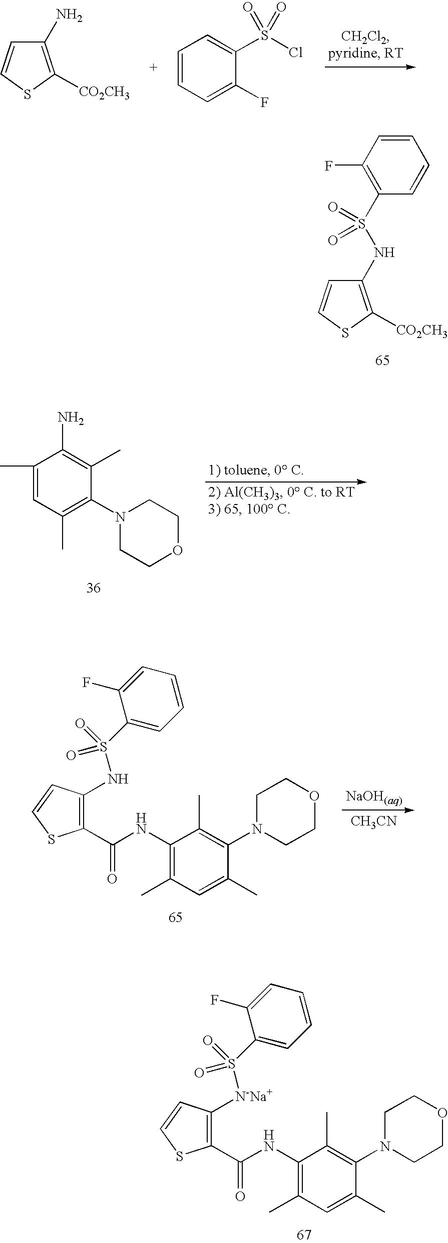

### I. Methyl 3-(2-fluorophenylsulfonamido)thiophene-2-carboxylate (65)

A mixture of methyl 3-aminothiophene-2-carboxylate (565 mg; 3.59 mmol), 2-fluorobenzene-1-sulfonyl chloride (0.22 mL; 1.66 mmol), and pyridine (0.7 mL; 8.65 mmol) in dichloromethane (10 mL) was stirred at room temperature overnight, poured into aqueous hydrochloric acid (50 mL; 1N) and then extracted with dichloromethane (2×15 mL). The dichloromethane layers were combined, washed with aqueous saturated sodium chloride, dried over sodium sulfate, filtered, and concentrated under reduced pressure. Recrystallization from ethyl acetate/hexanes gave the title compound as a light pink solid (543 mg).

### II. 3-(2-Fluorophenylsulfonamido)-N-[2,4,6-trimethyl-3-(morpholin-4-yl)phenyl]thiophene-2-carboxamide (66)

To a cooled (0° C.) solution of 36 (362 mg; 1.64 mmol) in toluene (4 mL) was added trimethylaluminum (1.1 mL; 2.2 mmol; 2M in toluene). After the reaction mixture was stirred at room temperature for 1 hour, a solution of 65 (461 mg; 1.46 mmol) in toluene (4 mL) was added via syringe. The reaction mixture was heated at 100° C. overnight, cooled to room temperature, poured into a flask containing ice (10 g) and aqueous hydrochloric acid (75 mL; 2N), and then extracted with ethyl acetate (3×25 mL). The organic layers were combined and washed successively with aqueous saturated sodium bicarbonate and aqueous saturated sodium chloride, dried over sodium sulfate, filtered, and concentrated under reduced pressure. The resulting crude was purified by automated silica gel column chromatography (Biotage®) eluting with 100% hexanes to 100% ethyl acetate gradient to give the title compound as a white solid (0.27 g).

### III. 3-(2-Fluorophenylsulfonamido)-N-[2,4,6-trimethyl-3-(morpholin-4-yl)phenyl]thiophene-2-carboxamide, Sodium Salt (67)

To a flask containing 66 (260 mg; 0.52 mmol) was added acetonitrile (2 mL) and aqueous sodium hydroxide (5 mL; 0.53 mmol; 0.105N). The reaction mixture was sonicated and gently heated to dissolve all solids. The resulting solution was filtered and then frozen in a dry ice/acetone bath and lyophilized to yield the title compound as an off-white solid (237 mg).

Mass Spectral Data (m/z): Measured (M−H)⁻=502.14; Calculated (M−H)⁻=502.13.

### Example 18

### Preparation of 3-(3-fluorophenylsulfonamido)-N-[2,4,6-trimethyl-3-(morpholin-4-yl)phenyl]thiophene-2-carboxamide, Sodium Salt (70)

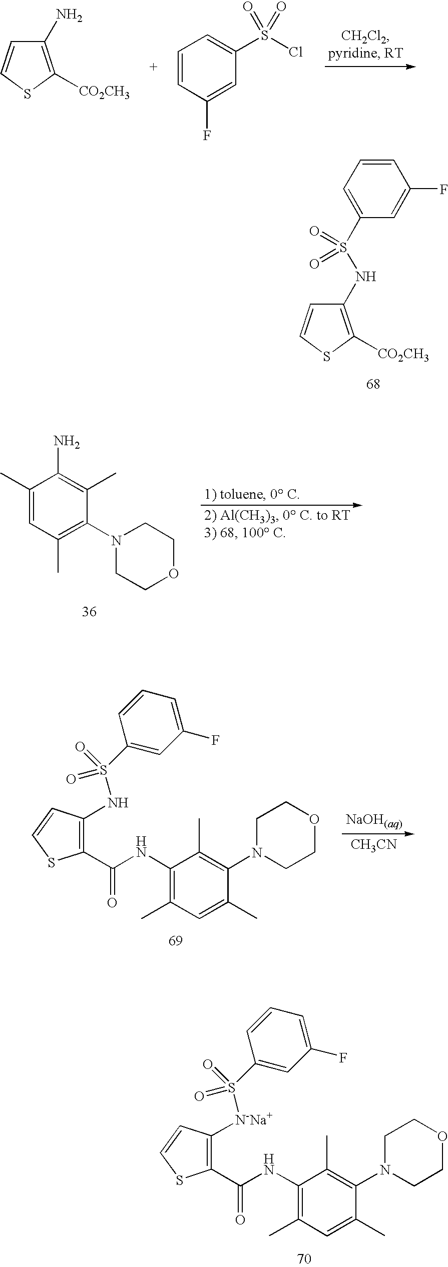

### I. Methyl 3-(3-fluorophenylsulfonamido)thiophene-2-carboxylate (68)

Synthesized as described for 65 using methyl 3-aminothiophene-2-carboxylate (560 mg; 3.56 mmol), 3-fluorobenzene-1-sulfonyl chloride (0.43 mL; 3.23 mmol), pyridine (1.3 mL; 16.1 mmol) and dichloromethane (10 mL). Recrystallization from ethyl acetate/hexanes gave the title compound a white solid (601 mg).

### II. 3-(3-Fluorophenylsulfonamido)-N-[2,4,6-trimethyl-3-(morpholin-4-yl)phenyl]thiophene-2-carboxamide (69)

Synthesized as described for 66 using 36 (556 mg; 2.52 mmol), 68 (596 mg; 1.88 mmol) in toluene (4 mL), trimethylaluminum (1.9 mL; 3.8 mmol; 2M in toluene) and toluene (5 mL). The resulting crude was purified by automated silica gel column chromatography (Biotage®) eluting with 100% hexanes to 100% ethyl acetate gradient to give the title compound as a white solid (0.90 g).

### III. 3-(3-Fluorophenylsulfonamido)-N-[2,4,6-trimethyl-3-(morpholin-4-yl)phenyl]thiophene-2-carboxamide, Sodium Salt (70)

Synthesized as described for 67 using 69 (0.38 g; 0.76 mmol), aqueous sodium hydroxide (7.2 mL; 0.76 mmol; 0.105N) and acetonitrile (2 mL). This afforded the title compound as a white solid (308 mg).

Mass Spectral Data (m/z): Measured (M−H)⁻=502.14; Calculated (M−H)⁻=502.13.

### Example 19

### Preparation of 3-[2-chloro-4-(trifluoromethoxy)phenylsulfonamido]-N-[2,4,6-trimethyl-3-(morpholin-4-yl)phenyl]thiophene-2-carboxamide, Sodium Salt (72)

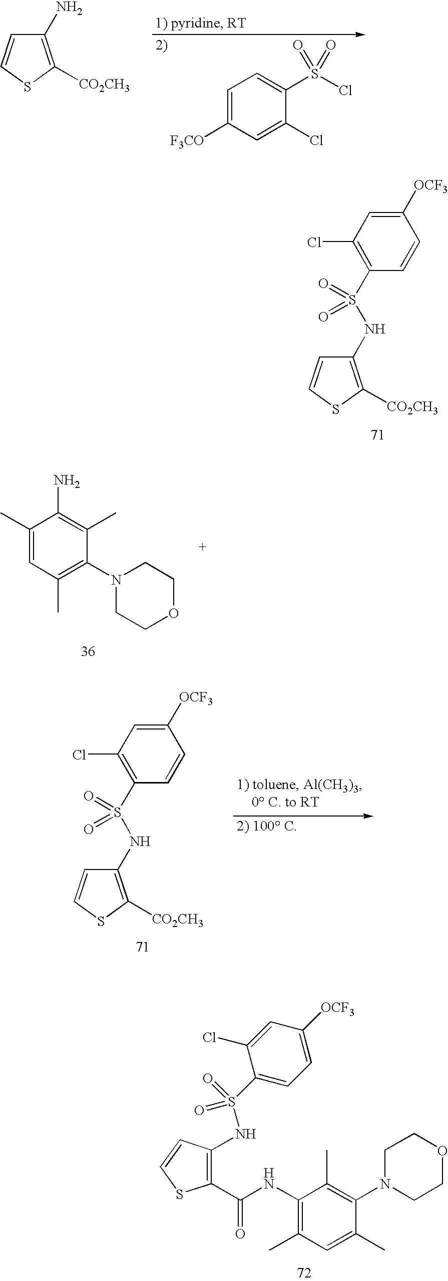

### I. Methyl 3-(2-chloro-4-(trifluoromethoxy)phenylsulfonamido)thiophene-2-carboxylate (71)

To a solution of methyl 3-aminothiophene-2-carboxylate (0.30 g; 1.9 mmol) in pyridine (2 mL) was added 2-chloro-4-(trifluoromethoxy)benzene-1-sulfonyl chloride. The reaction mixture was stirred overnight at room temperature, and then diluted with water. The resulting precipitate was collected by filtration to give the title compound as a yellow solid (0.61 g).

### II. 3-[2-Chloro-4-(trifluoromethoxy)phenylsulfonamido]-N-[2,4,6-trimethyl-3-(morpholin-4-yl)phenyl]thiophene-2-carboxamide (72)

To a cooled (0° C.) solution of 36 (200 mg; 0.91 mmol) and 71 (333 mg; 0.80 mmol) in toluene (4 mL) was added trimethylaluminum (0.7 mL; 1.4 mmol; 2M in toluene). The reaction mixture was stirred at 0° C. for 15 minutes, warmed to room temperature and then heated at 100° C. overnight. After cooling to room temperature, the reaction mixture was poured into aqueous hydrochloric acid (100 mL; 2N), and then extracted with ethyl acetate (3×20 mL). The organic layers were combined and washed successively with aqueous saturated ammonium chloride, water, and aqueous saturated sodium chloride, dried over sodium sulfate, filtered, and concentrated under reduced pressure. The resulting crude product was purified by automated silica gel column chromatography (Biotage®) to give the title compound as a pale yellow solid (180 mg).

Mass Spectral Data (m/z): Measured (M+H)⁺=604.14; Calculated (M+H)⁺=604.10.

### Example 20

### Preparation of 3-(4-ethylphenylsulfonamido)-N-[2,4,6-trimethyl-3-(morpholin-4-yl)phenyl]thiophene-2-carboxamide, Sodium Salt (75)

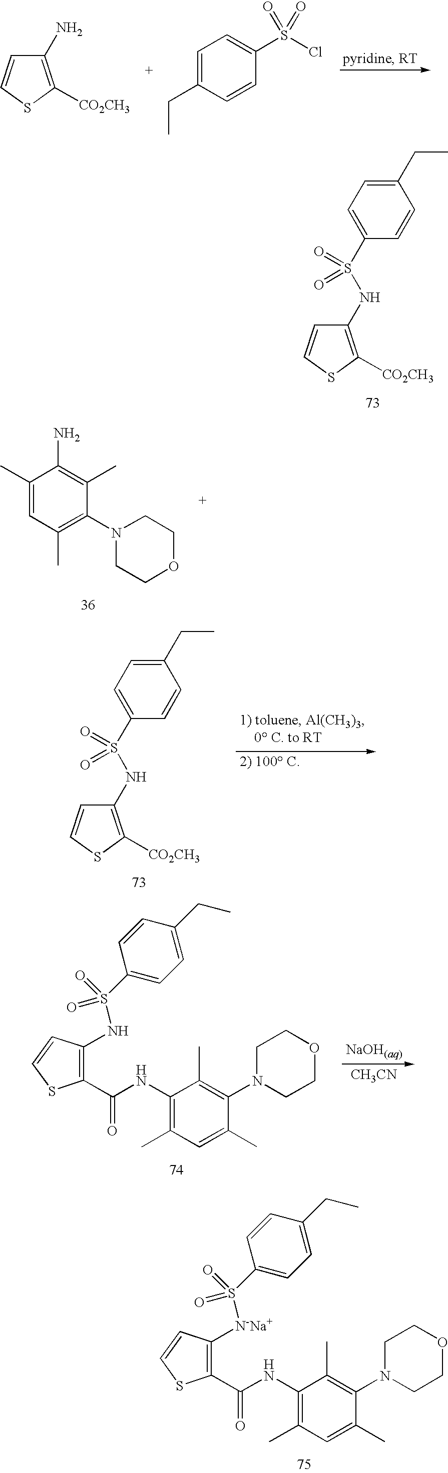

### I. Methyl 3-(4-ethylphenylsulfonamido)thiophene-2-carboxylate (73)

A mixture of methyl 3-aminothiophene-2-carboxylate (0.705 g; 4.48 mmol) and 4-ethylbenzene-1-sulfonyl chloride (0.91 g; 4.4 mmol) in pyridine (5 mL) was stirred at room temperature for 4 hours, poured into aqueous hydrochloric acid (150 mL; 2N), and then extracted with dichloromethane (2×30 mL). The dichloromethane layers were combined, dried over sodium sulfate, filtered, and concentrated under reduced pressure to give the title compound as a pink oil (1.50 g).

### II. 3-(4-Ethylphenylsulfonamido)-N-[2,4,6-trimethyl-3-(morpholin-4-yl)phenyl]thiophene-2-carboxamide (74)

Synthesized as described for 72 using 36 (1.10 g; 4.99 mmol), 73 (1.50 g; 4.60 mmol), trimethylaluminum (5.0 mL; 10 mmol; 2M in toluene) and toluene (20 mL). The resulting crude product was purified by automated silica gel column chromatography (Biotage®) eluting with an ethyl acetate/hexanes gradient to give the title compound as an off-white solid (0.60 g).

### III. 3-(4-Ethylphenylsulfonamido)-N-[2,4,6-trimethyl-3-(morpholin-4-yl)phenyl]thiophene-2-carboxamide, Sodium Salt (75)

To a flask containing 74 (0.60 g; 1.17 mmol) were added aqueous sodium hydroxide (12 mL; 1.23 mmol; 0.1023N) and acetonitrile (3 mL). The reaction mixture was gently heated to dissolve all solids. The resulting solution was filtered and then frozen in a dry ice/acetone bath and lyophilized to yield the title compound as an off-white solid (607 mg).

Mass Spectral Data (m/z): Measured (M−H)⁻=512.17; Calculated (M−H)⁻=512.17.

### Example 21

### Preparation of 3-(4-methylphenylsulfonamido)-N-[2,4,6-trimethyl-3-(morpholin-4-yl)phenyl]thiophene-2-carboxamide, Sodium Salt (77)

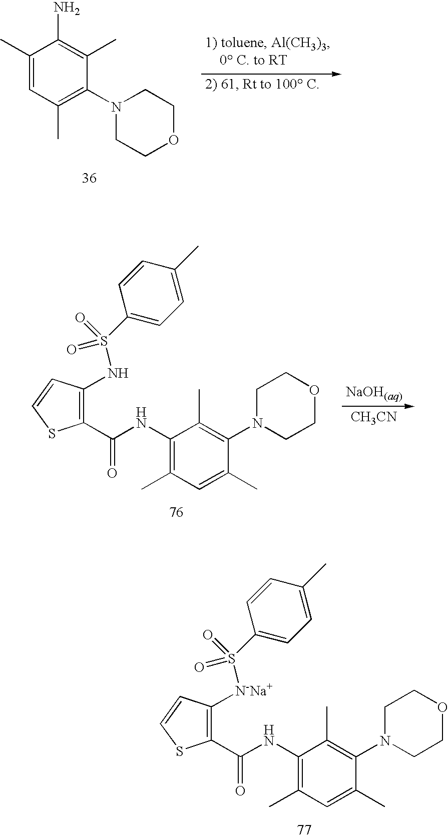

### I. 3-(4-Methylphenylsulfonamido)-N-[2,4,6-trimethyl-3-(morpholin-4-yl)phenyl]thiophene-2-carboxamide (76)

To an oven-dried 100 mL round bottom flask containing a stir bar, cooled under vacuum and purged with nitrogen, were added 36 (2.20 g; 9.98 mmol) and anhydrous toluene (20 mL). The vessel was placed under vacuum and purged with nitrogen (repeated three times) then cooled to 0° C. in an ice bath. and trimethylaluminum (10 mL; 20 mmol; 2M in toluene) was added dropwise. The reaction was stirred an additional 15 minutes at 0° C., warmed to room temperature and stirred for 30 minutes. To the resulting mixture, 61 (2.95 g, 9.47 mmol) was added in a single portion. The reaction mixture was then heated to 100° C. for 24 hours, was allowed to cool to room temperature and was then poured into crushed ice (50 g) and aqueous hydrochloric acid (200 mL; 2N). The resulting mixture was extracted with isopropyl acetate (3×40 ml). The organic extracts were combined and washed with brine and the layers were separated. To the organic layer, finely powdered charcoal (2.0 g) was added and the resulting mixture was stirred for 40 minutes with intermittent gentle heating. The mixture was filtered through Celite® and the filtrate was concentrated under reduced pressure. The crude product was taken up in a mixture of isopropyl acetate (50 mL) and hexanes (50 mL) and the mixture was stirred overnight then sonicated for 15 minutes and filtered to give the title compounds as a white solid (4.17 g).

### II. 3-(4-Methylphenylsulfonamido)-N-[2,4,6-trimethyl-3-(morpholin-4-yl)phenyl]thiophene-2-carboxamide, Sodium Salt (77)

A suspension of 76 (1.8 g; 3.6 mmol) in aqueous sodium hydroxide (8.0 mL; 4.0 mmol; 0.5M) was stirred at 80° C. until a clear solution was obtained. The solution was allowed to cool to room temperature and the resulting crystals were collected by filtration and dried under vacuum to give the title compound as an off-white solid (1.974 g).

Mass Spectral Data (m/z): Measured (M−H)⁻=498.20; Calculated (M−H)⁻=498.15.

### Example 22

### Preparation of 3-[4-(trifluoromethyl)phenylsulfonamido]-N-[2,4,6-trimethyl-3-(morpholin-4-yl)phenyl]thiophene-2-carboxamide, Sodium Salt (80)

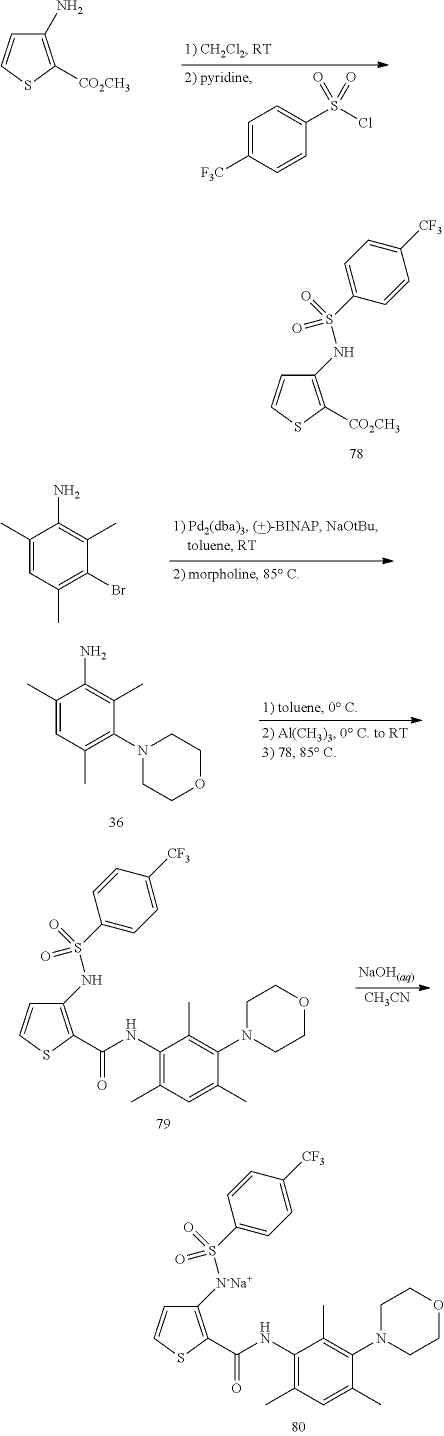

### I. Methyl 3-[4-(trifluoromethyl)phenylsulfonamido]thiophene-2-carboxylate (78)

To a solution of methyl 3-aminothiophene-2-carboxylate (0.25 g; 1.59 mmol) in dichloromethane (4 mL), was added 4-(trifluoromethyl)benzene-1-sulfonyl chloride (0.43 g; 1.76 mmol) and pyridine (0.3 mL; 3.7 mmol). After stirring overnight at room temperature, the reaction was quenched by addition of aqueous hydrochloric acid (100 mL; 1N) and then extracted with dichloromethane (3×30 mL). The organic phases were combined, washed successively with aqueous saturated sodium bicarbonate and aqueous saturated sodium chloride, dried over sodium sulfate, and filtered. The filtrate was concentrated under reduced pressure to give the title product as a red solid (0.484 g).

### II. 2,4,6-Trimethyl-3-(morpholin-4-yl)aniline (36)

To a suspension of 3-bromo-2,4,6-trimethylaniline (5.00 g; 23.4 mmol), sodium tert-butoxide (4.45 g; 46.3 mmol), bis(dibenzylideneacetone)palladium(0) (0.43 g; 0.47 mmol) and (±)-BINAP (0.56 g; 0.89 mmol) in toluene (100 mL) was added morpholine (2.60 mL; 29.7 mmol) via syringe. The reaction mixture was heated at 85° C. overnight under a nitrogen atmosphere. Additional morpholine (4 mL) was added and heating was continued at 85° C. for an additional 24 hours. After cooling to room temperature, the reaction mixture was diluted with aqueous hydrochloric acid (100 mL; 2N) and then washed with diethyl ether. The aqueous layer was separated and basified by addition of sodium bicarbonate and then extracted with ethyl acetate (4×50 mL). All ethyl acetate layers were combined, dried over sodium sulfate, filtered, and concentrated under reduced pressure. The resulting crude product was filtered through a short silica gel column and then triturated from hexanes to give the title compound as a light yellow solid (0.30 g).

### III. 3-[4-(Trifluoromethyl)phenylsulfonamido]-N-[2,4,6-trimethyl-3-(morpholin-4-yl)phenyl]thiophene-2-carboxamide (79)

To a cooled (0° C.) solution of 36 (256 mg; 1.16 mmol) in toluene (3 mL) was added trimethylaluminum (1.7 mL; 3.4 mmol; 2M in hexanes). After the reaction mixture was stirred at 0° C. for 15 minutes and then at room temperature for 45 minutes, a solution of 78 (463 mg; 1.27 mmol) in toluene (3.5 mL) was added. The reaction mixture was heated at 85° C. overnight, cooled to room temperature, poured into a flask containing ice (25 g) and aqueous hydrochloric acid (50 mL; 1N), and then extracted with ethyl acetate (3×50 mL). The organic layers were combined and washed with aqueous saturated sodium chloride, dried over sodium sulfate, filtered, and concentrated under reduced pressure. The resulting crude product recrystallized from ethyl acetate/hexanes to give the title compound (0.447 g).

### IV. 3-[4-(Trifluoromethyl)phenylsulfonamido]-N-[2,4,6-trimethyl-3-(morpholin-4-yl)phenyl]thiophene-2-carboxamide, Sodium Salt (80)

To a solution of 79 (447 mg; 0.81 mmol) in acetonitrile (7 mL) was added aqueous sodium hydroxide (8.0 mL; 0.84 mmol; 0.105N). The resulting solution was filtered and then frozen in a dry ice/acetone bath and lyophilized to yield the title compound as a pale orange solid (400 mg).

Mass Spectral Data (m/z): Measured (M+H)⁺=554.15; Calculated (M+H)⁺=554.14.

### Example 23

### Preparation of 3-[3-(trifluoromethyl)phenylsulfonamido]-N-[2,4,6-trimethyl-3-(morpholin-4-yl)phenyl]thiophene-2-carboxamide, Sodium Salt (83)

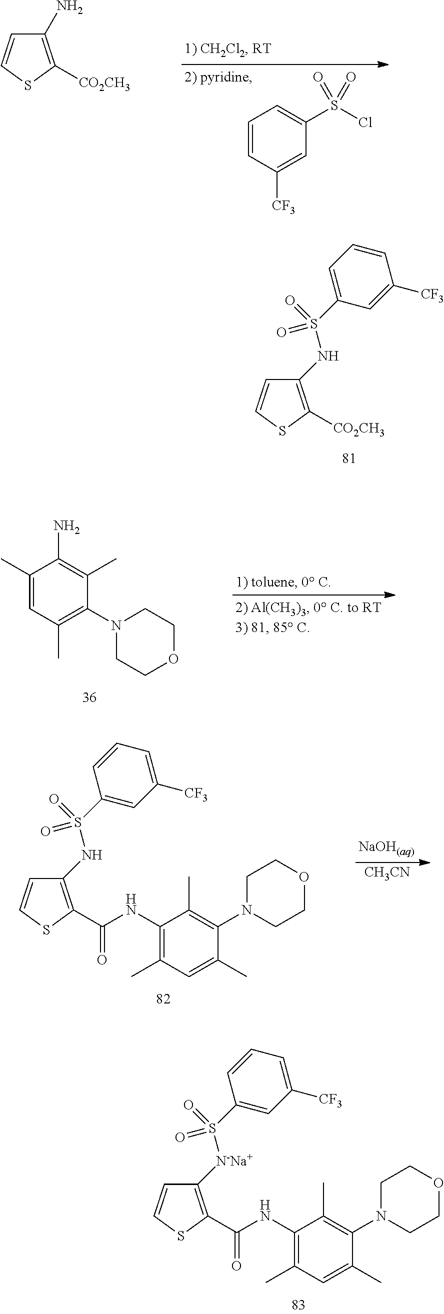

### I. Methyl 3-[3-(trifluoromethyl)phenylsulfonamido]thiophene-2-carboxylate (81)

Synthesized as described for 78 using methyl 3-aminothiophene-2-carboxylate (0.250 g; 1.59 mmol), 3-(trifluoromethyl)benzene-1-sulfonyl chloride (0.28 mL; 1.75 mmol), pyridine (0.30 mL; 3.71 mmol) and dichloromethane (4 mL). This afforded the title compound a light tan solid (558 mg).

### II. 3-[3-(Trifluoromethyl)phenylsulfonamido]-N-[2,4,6-trimethyl-3-(morpholin-4-yl)phenyl]thiophene-2-carboxamide (82)

Synthesized as described for 79 using 36 (255 mg; 1.16 mmol), toluene (3 mL), trimethylaluminum (1.7 mL; 3.4 mmol; 2M in hexanes) and a solution of 81 (504 mg; 1.38 mmol) in toluene (3.5 mL). The resulting crude product was recrystallized from ethyl acetate/hexanes to give the title compound (434 mg).

### III. 3-[3-(Trifluoromethyl)phenylsulfonamido]-N-[2,4,6-trimethyl-3-(morpholin-4-yl)phenyl]thiophene-2-carboxamide, Sodium Salt (83)

Synthesized as described for 80 using 82 (434 mg; 0.78 mmol), aqueous sodium hydroxide (7.7 mL; 0.81 mmol; 0.105N) and acetonitrile (11 mL). This afforded the title compound as a pink solid (425 mg).

Mass Spectral Data (m/z): Measured (M+H)⁺=554.18; Calculated (M+H)⁺=554.14.

### Example 24

### Preparation of 3-[3-(trifluoromethoxy)phenylsulfonamido]-N-[2,4,6-trimethyl-3-(morpholin-4-yl)phenyl]thiophene-2-carboxamide, Sodium Salt (86)

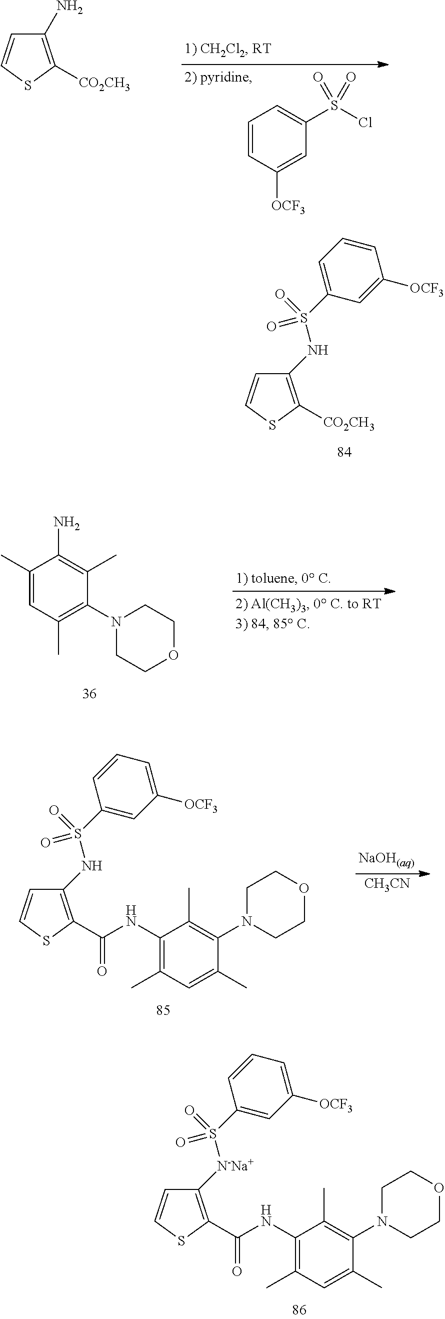

### I. Methyl 3-[3-(trifluoromethoxy)phenylsulfonamido]thiophene-2-carboxylate (84)

Synthesized as described for 78 using methyl 3-aminothiophene-2-carboxylate (0.75 g; 4.77 mmol), 3-(trifluoromethoxy)benzene-1-sulfonyl chloride (1.0 g; 3.83 mmol), pyridine (1.25 mL; 15.5 mmol) and dichloromethane (10 mL). The resulting crude product was purified by automated silica gel column chromatography (Biotage®) eluting with an ethyl acetate/hexanes gradient (0% to 50%) to give the title compound as a white solid (1.10 g).

### II. 3-[3-(Trifluoromethoxy)phenylsulfonamido]-N-[2,4,6-trimethyl-3-(morpholin-4-yl)phenyl]thiophene-2-carboxamide (85)

To a cooled (0° C.) solution of 36 (229 mg; 1.04 mmol) in toluene (4 mL) was added trimethylaluminum (2.0 mL; 4.0 mmol; 2M in hexanes). After the reaction mixture was stirred at room temperature for 45 minutes, 84 (439 mg; 1.15 mmol) was added. The reaction mixture was heated at 85° C. overnight, cooled to room temperature, poured into a flask containing ice (25 g) and aqueous hydrochloric acid (75 mL; 2N), and then extracted with ethyl acetate (3×30 mL). The organic layers were combined, dried over sodium sulfate, filtered, and concentrated under reduced pressure. The resulting crude product was purified by column chromatography on silica gel, eluting with 45% ethyl acetate/hexanes to give the title compound (110 mg).

### III. 3-[3-(Trifluoromethoxy)phenylsulfonamido]-N-[2,4,6-trimethyl-3-(morpholin-4-yl)phenyl]thiophene-2-carboxamide, Sodium Salt (86)

Synthesized as described for 80 using 85 (110 mg; 0.19 mmol), aqueous sodium hydroxide (1.9 mL; 0.20 mmol; 0.105N) and acetonitrile (3 mL). This afforded the title compound as a pale yellow solid (104 mg).

Mass Spectral Data (m/z): Measured (M+H)⁺=570.15; Calculated (M+H)⁺=570.13.

### Example 25

### Preparation of 3-[4-(trifluoromethoxy)phenylsulfonamido]-N-[2,4,6-trimethyl-3-(morpholin-4-yl)phenyl]thiophene-2-carboxamide, Sodium Salt (89)

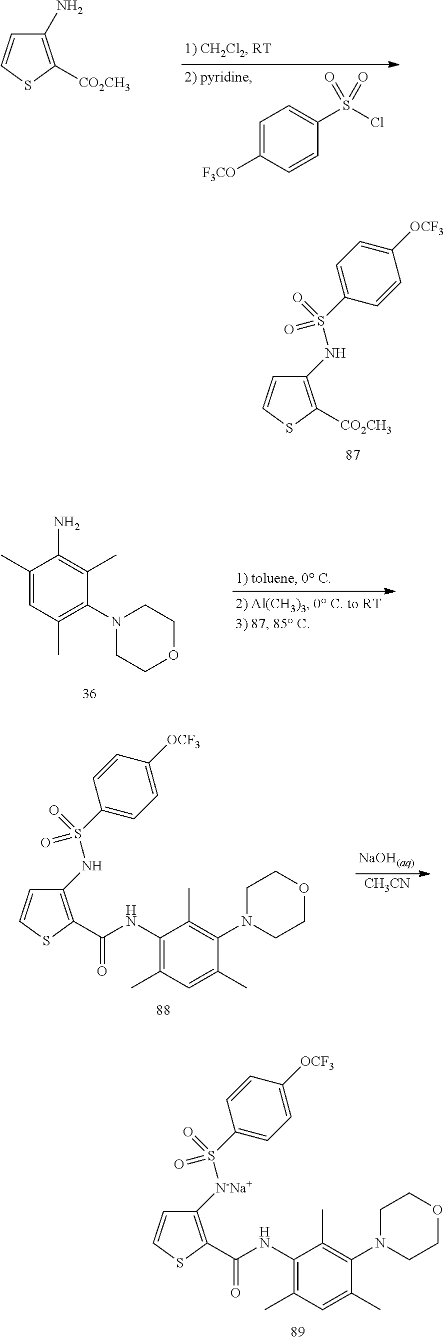

### I. Methyl 3-[4-(trifluoromethoxy)phenylsulfonamido]thiophene-2-carboxylate (87)

Synthesized as described for 78 using methyl 3-aminothiophene-2-carboxylate (0.71 g; 4.51 mmol), 4-(trifluoromethoxy)benzene-1-sulfonyl chloride (1.0 g; 3.83 mmol), pyridine (1.25 mL; 15.5 mmol) and dichloromethane (10 mL). The resulting crude product was purified by automated silica gel column chromatography (Biotage®) eluting with an ethyl acetate/hexanes gradient (0% to 50%) to give the title compound as a white solid (1.36 g).

### II. 3-[4-(Trifluoromethoxy)phenylsulfonamido]-N-[2,4,6-trimethyl-3-(morpholin-4-yl)phenyl]thiophene-2-carboxamide (88)

Synthesized as described for 85 using 36 (225 mg; 1.02 mmol), 87 (430 mg; 1.13 mmol), trimethylaluminum (2.0 mL; 4.0 mmol; 2M in hexanes) and toluene (4 mL). The resulting crude product was purified by recrystallization from ethyl acetate/hexanes followed by column chromatography on silica gel, eluting with an ethyl acetate/hexanes gradient (25% to 45%) to give the title compound (102 mg).

### III. 3-[4-(Trifluoromethoxy)phenylsulfonamido]-N-[2,4,6-trimethyl-3-(morpholin-4-yl)phenyl]thiophene-2-carboxamide, Sodium Salt (89)

Synthesized as described for 80 using 88 (102 mg; 0.18 mmol), aqueous sodium hydroxide (1.75 mL; 0.18 mmol; 0.105N) and acetonitrile (2.5 mL). This afforded the title compound as an off-white solid (106 mg).

Mass Spectral Data (m/z): Measured (M−H)⁻=568.12; Calculated (M−H)⁻=568.12.

### Example 26

### Preparation of 3-(4-methoxyphenylsulfonamido)-N-[2,4,6-trimethyl-3-(morpholin-4-yl)phenyl]thiophene-2-carboxamide, Sodium Salt (92)

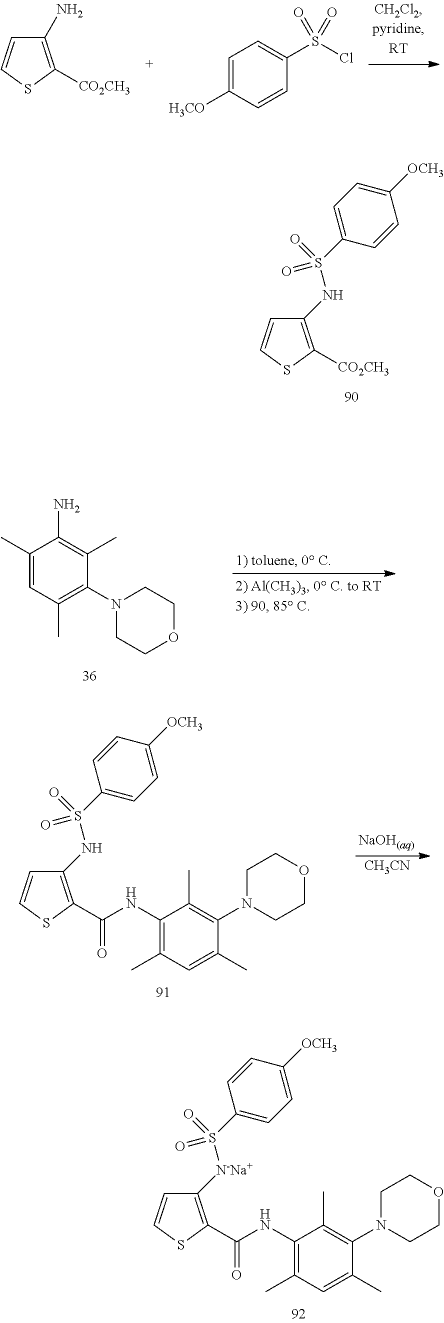

### I. Methyl 3-(4-methoxyphenylsulfonamido)thiophene-2-carboxylate (90)

Synthesized as described for 65 using methyl 3-aminothiophene-2-carboxylate (0.74 g; 4.70 mmol), 4-methoxybenzene-1-sulfonyl chloride (0.92 g; 4.45 mmol), pyridine (1.75 mL; 21.6 mmol) and dichloromethane (15 mL). Recrystallization from ethyl acetate/hexanes gave the title compound a light pink solid (928 mg).

### II. 3-(4-Methoxyphenylsulfonamido)-N-[2,4,6-trimethyl-3-(morpholin-4-yl)phenyl]thiophene-2-carboxamide (91)

Synthesized as described for 85 using 36 (259 mg; 1.18 mmol), 90 (427 mg; 1.30 mmol), trimethylaluminum (2.4 mL; 4.8 mmol; 2M in hexanes) and toluene (3 mL). The resulting crude product purified by automated silica gel column chromatography (Biotage®) eluting with an ethyl acetate/hexanes gradient (8% to 70%) to give the title compound (357 mg).

### III. 3-(4-Methoxyphenylsulfonamido)-N-[2,4,6-trimethyl-3-(morpholin-4-yl)phenyl]thiophene-2-carboxamide, Sodium Salt (92)

Synthesized as described for 80 using 91 (357 mg; 0.69 mmol), aqueous sodium hydroxide (6.8 mL; 0.71 mmol; 0.105N) and acetonitrile (5 mL). This afforded the title compound as a yellow solid (367 mg).

Mass Spectral Data (m/z): Measured (M−H)⁻=514.15; Calculated (M−H)⁻=514.15.

### Example 27

### Preparation of 3-(2,4-difluorophenylsulfonamido)-N-[2,4,6-trimethyl-3-(morpholin-4-yl)phenyl]thiophene-2-carboxamide, Sodium Salt (95)

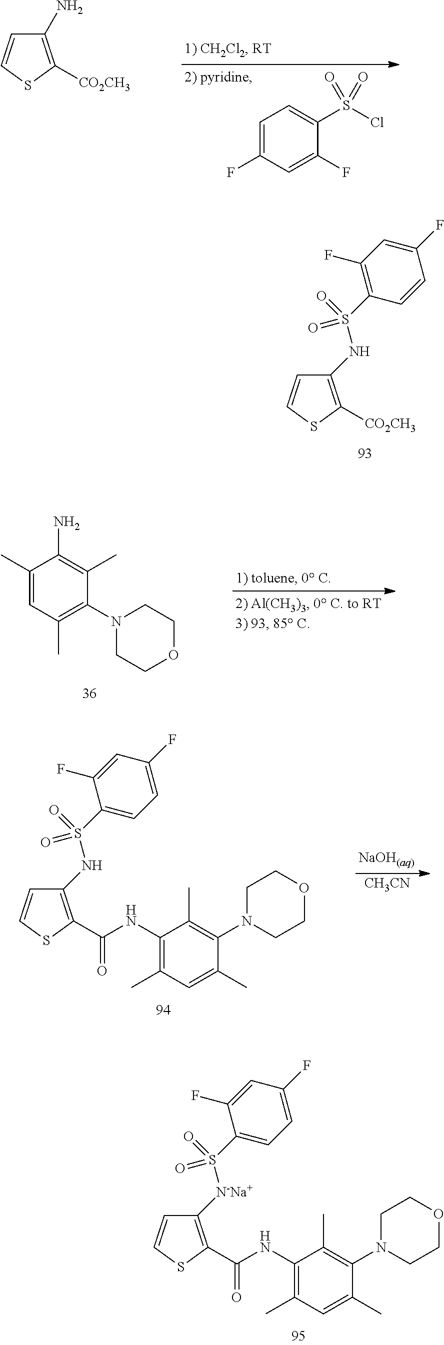

### I. Methyl 3-(2,4-difluorophenylsulfonamido)thiophene-2-carboxylate (93)

Synthesized as described for 78 using methyl 3-aminothiophene-2-carboxylate (259 mg; 1.65 mmol), 2,4-difluorobenzene-1-sulfonyl chloride (0.25 mL; 1.86 mmol), pyridine (0.4 mL; 4.95 mmol) and dichloromethane (2 mL). This afforded the title compound (538 mg).

### II. 3-(2,4-Difluorophenylsulfonamido)-N-[2,4,6-trimethyl-3-(morpholin-4-yl)phenyl]thiophene-2-carboxamide (94)

Synthesized as described for 85 using 36 (256 mg; 1.16 mmol), 93 (432 mg; 1.30 mmol), trimethylaluminum (2.5 mL; 5.0 mmol; 2M in hexanes) and toluene (2 mL). The resulting crude product was purified by automated silica gel column chromatography (Biotage®) eluting with an ethyl acetate/hexanes gradient (8% to 75%) to give the title compound (235 mg).

### III. 3-(2,4-Difluorophenylsulfonamido)-N-[2,4,6-trimethyl-3-(morpholin-4-yl)phenyl]thiophene-2-carboxamide, Sodium Salt (95)

Synthesized as described for 80 using 94 (247 mg; 0.47 mmol), aqueous sodium hydroxide (4.75 mL; 0.50 mmol; 0.105N) and acetonitrile (6.5 mL). This afforded the title compound as a yellow solid (248 mg).

Mass Spectral Data (m/z): Measured (M+H)⁺=522.14; Calculated (M+H)⁺=522.13.

### Example 28

### Preparation of 3-(2-chlorophenylsulfonamido)-N-[2,4,6-trimethyl-3-(morpholin-4-yl)phenyl]thiophene-2-carboxamide, Sodium Salt (98)

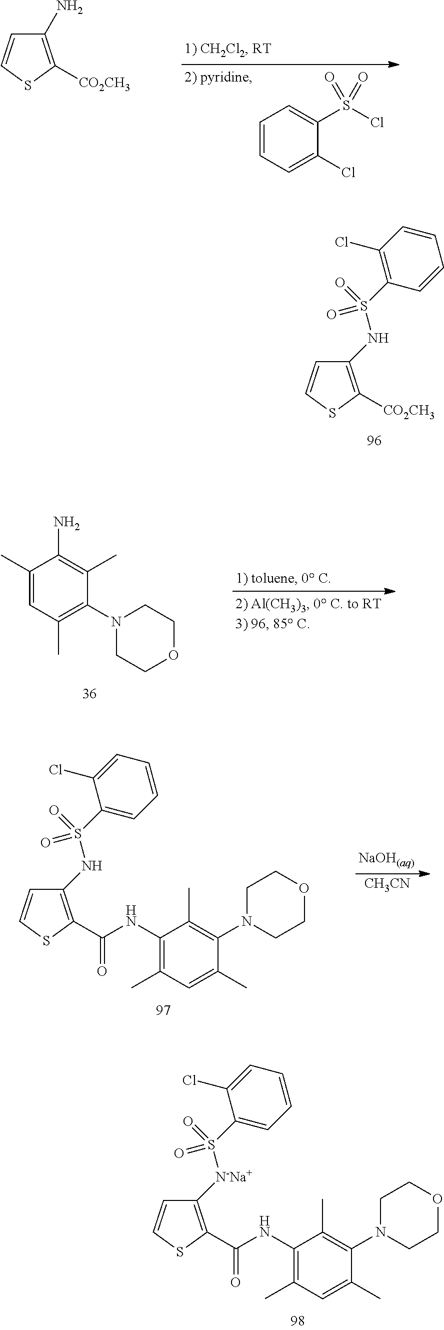

### I. Methyl 3-(2-chlorophenylsulfonamido)thiophene-2-carboxylate (96)

Synthesized as described for 78 using methyl 3-aminothiophene-2-carboxylate (255 mg; 1.62 mmol), 2-chlorobenzene-1-sulfonyl chloride (398 mg; 1.89 mmol), pyridine (0.4 mL; 4.95 mmol) and dichloromethane (3 mL). This afforded the title compound as a red solid (466 mg).

### II. 3-(2-Chlorophenylsulfonamido)-N-[2,4,6-trimethyl-3-(morpholin-4-yl)phenyl]thiophene-2-carboxamide (97)

Synthesized as described for 85 using 36 (258 mg; 1.17 mmol), 96 (440 mg; 1.33 mmol), trimethylaluminum (2.4 mL; 4.8 mmol; 2M in hexanes) and toluene (2 mL). The resulting crude product was purified by column chromatography on silica gel, eluting with an ethyl acetate/hexanes gradient (15% to 75%) to give the title compound (408 mg).

### III. 3-(2-Chlorophenylsulfonamido)-N-[2,4,6-trimethyl-3-(morpholin-4-yl)phenyl]thiophene-2-carboxamide, Sodium Salt (98)

Synthesized as described for 80 using 97 (389 mg; 0.75 mmol), aqueous sodium hydroxide (7.5 mL; 0.79 mmol; 0.105N) and acetonitrile (9 mL). This afforded the title compound as a yellow solid (382 mg).

Mass Spectral Data (m/z): Measured (M+H)⁺=520.10; Calculated (M+H)⁺=520.11.

### Example 29

### Preparation of 3-(2,4-dimethylphenylsulfonamido)-N-[2,4,6-trimethyl-3-(morpholin-4-yl)phenyl]thiophene-2-carboxamide, Sodium Salt (101)

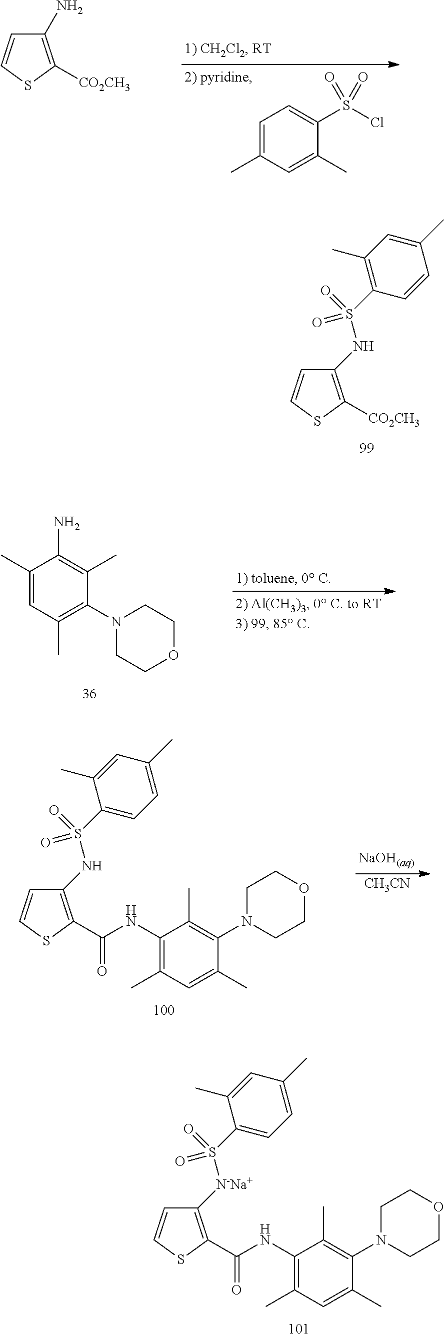

### I. Methyl 3-(2,4-dimethylphenylsulfonamido)thiophene-2-carboxylate (99)

Synthesized as described for 78 using methyl 3-aminothiophene-2-carboxylate (253 mg; 1.61 mmol), 2,4-dimethylbenzene-1-sulfonyl chloride (373 mg; 1.82 mmol), pyridine (0.4 mL; 4.95 mmol) and dichloromethane (2 mL). This afforded the title compound (482 mg).

### II. 3-(2,4-Dimethylphenylsulfonamido)-N-[2,4,6-trimethyl-3-(morpholin-4-yl)phenyl]thiophene-2-carboxamide (100)

Synthesized as described for 85 using 36 (255 mg; 1.16 mmol), 99 (423 mg; 1.30 mmol), trimethylaluminum (2.5 mL; 5.0 mmol; 2M in hexanes) and toluene (2 mL). The resulting crude product was purified by column chromatography on silica gel, eluting with an ethyl acetate/hexanes gradient (8% to 70%) to give the title compound (318 mg).

### III. 3-(2,4-Dimethylphenylsulfonamido)-N-[2,4,6-trimethyl-3-(morpholin-4-yl)phenyl]thiophene-2-carboxamide, Sodium Salt (101)

Synthesized as described for 80 using 100 (314 mg; 0.61 mmol), aqueous sodium hydroxide (6.2 mL; 0.65 mmol; 0.105N) and acetonitrile (8 mL). This afforded the title compound as a yellow solid (299 mg).

Mass Spectral Data (m/z): Measured (M+H)⁺=514.19; Calculated (M+H)⁺=514.18.

### Example 30

### Preparation of 3-(2-chloro-4-fluorophenylsulfonamido)-N-[2,4,6-trimethyl-3-(morpholin-4-yl)phenyl]thiophene-2-carboxamide, Sodium Salt (104)

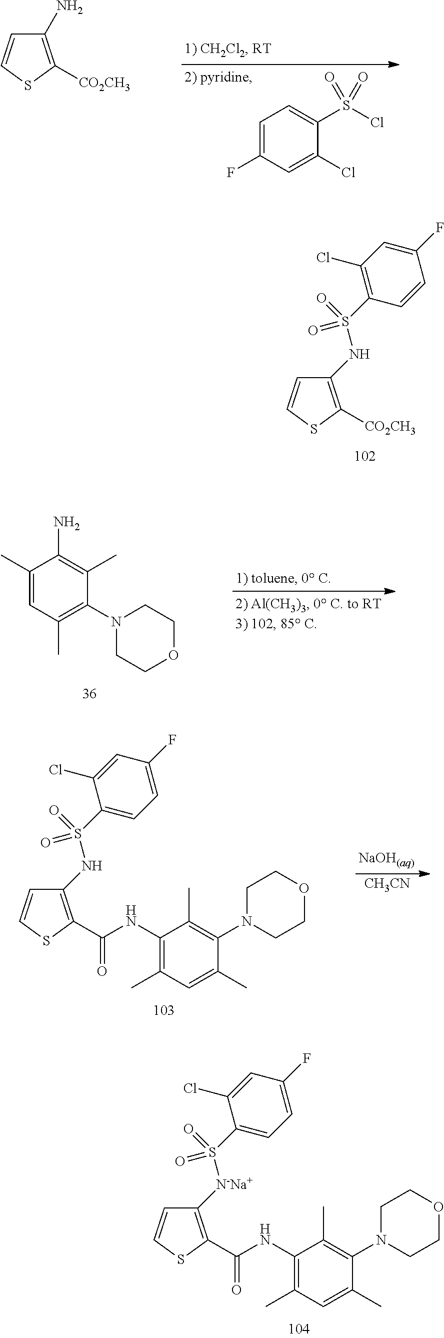

### I. Methyl 3-(2-chloro-4-fluorophenylsulfonamido)thiophene-2-carboxylate (102)

Synthesized as described for 78 using methyl 3-aminothiophene-2-carboxylate (256 mg; 1.63 mmol), 2-chloro-4-fluorobenzene-1-sulfonyl chloride (0.27 mL; 1.85 mmol), pyridine (0.41 mL; 5.07 mmol) and dichloromethane (2 mL). This afforded the title compound (535 mg).

### II. 3-(2-Chloro-4-fluorophenylsulfonamido)-N-[2,4,6-trimethyl-3-(morpholin-4-yl)phenyl]thiophene-2-carboxamide (103)

Synthesized as described for 85 using 36 (211 mg; 0.96 mmol), 102 (373 mg; 1.07 mmol), trimethylaluminum (2.5 mL; 5.0 mmol; 2M in hexanes) and toluene (2 mL). The resulting crude product was purified by automated silica gel column chromatography (Biotage®) eluting with an ethyl acetate/hexanes gradient (10% to 80%) to give the title compound (46 mg).

### III. 3-(2-Chloro-4-fluorophenylsulfonamido)-N-[2,4,6-trimethyl-3-(morpholin-4-yl)phenyl]thiophene-2-carboxamide, Sodium Salt (104)

Synthesized as described for 80 using 103 (45 mg; 0.084 mmol), aqueous sodium hydroxide (0.85 mL; 0.089 mmol; 0.105N) and acetonitrile (3 mL). This afforded the title compound as a tan solid (45 mg).

Mass Spectral Data (m/z): Measured (M+H)⁺=538.11; Calculated (M+H)⁺=538.10.

### Example 31

### Preparation of 3-(4-fluoro-2-methylphenylsulfonamido)-N-[2,4,6-trimethyl-3-(morpholin-4-yl)phenyl]thiophene-2-carboxamide, Sodium Salt (107)

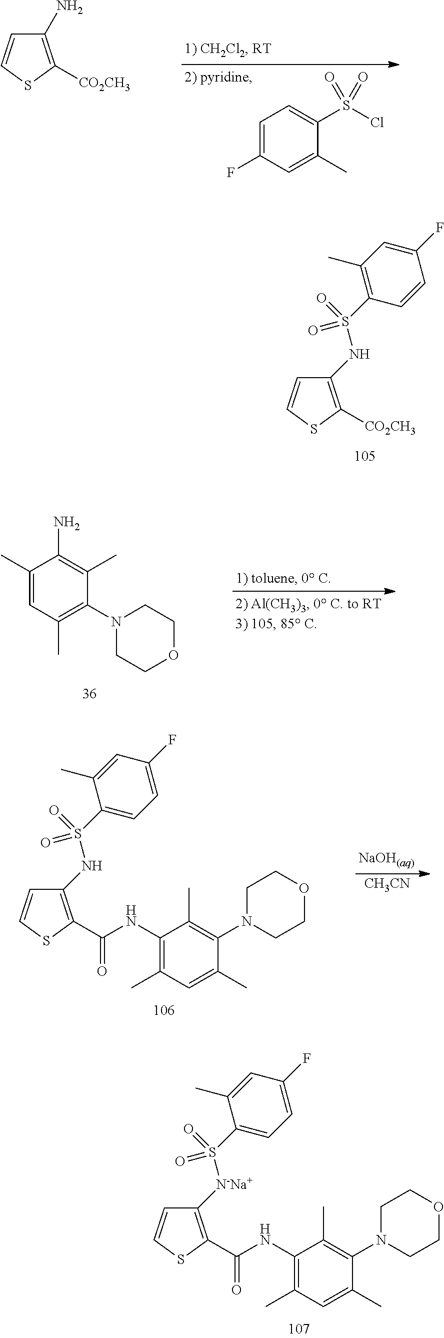

### I. Methyl 3-(4-fluoro-2-methylphenylsulfonamido)thiophene-2-carboxylate (105)

Synthesized as described for 78 using methyl 3-aminothiophene-2-carboxylate (253 mg; 1.61 mmol), 4-fluoro-2-methylbenzene-1-sulfonyl chloride (0.27 mL; 1.85 mmol), pyridine (0.41 mL; 5.07 mmol), and dichloromethane (2 mL). This afforded the title compound (580 mg).

### II. 3-(4-Fluoro-2-methylphenylsulfonamido)-N-[2,4,6-trimethyl-3-(morpholin-4-yl)phenyl]thiophene-2-carboxamide (106)

Synthesized as described for 85 using 36 (210 mg; 0.95 mmol), 105 (404 mg; 1.23 mmol), trimethylaluminum (2.5 mL; 5.0 mmol; 2M in hexanes) and toluene (2 mL). The resulting crude product was purified by column chromatography on silica gel to give the title compound (337 mg).

### III. 3-(4-Fluoro-2-methylphenylsulfonamido)-N-[2,4,6-trimethyl-3-(morpholin-4-yl)phenyl]thiophene-2-carboxamide, Sodium Salt (107)

Synthesized as described for 80 using 106 (332 mg; 0.64 mmol), aqueous sodium hydroxide (6.5 mL; 0.68 mmol; 0.105N) and acetonitrile (5 mL). This afforded the title compound as a yellow solid (339 mg).

Mass Spectral Data (m/z): Measured (M+H)⁺=518.16; Calculated (M+H)⁺=518.16.

### Example 32

### Preparation of 3-(4-chloro-2-fluorophenylsulfonamido)-N-[2,4,6-trimethyl-3-(morpholin-4-yl)phenyl]thiophene-2-carboxamide, Sodium Salt (110)

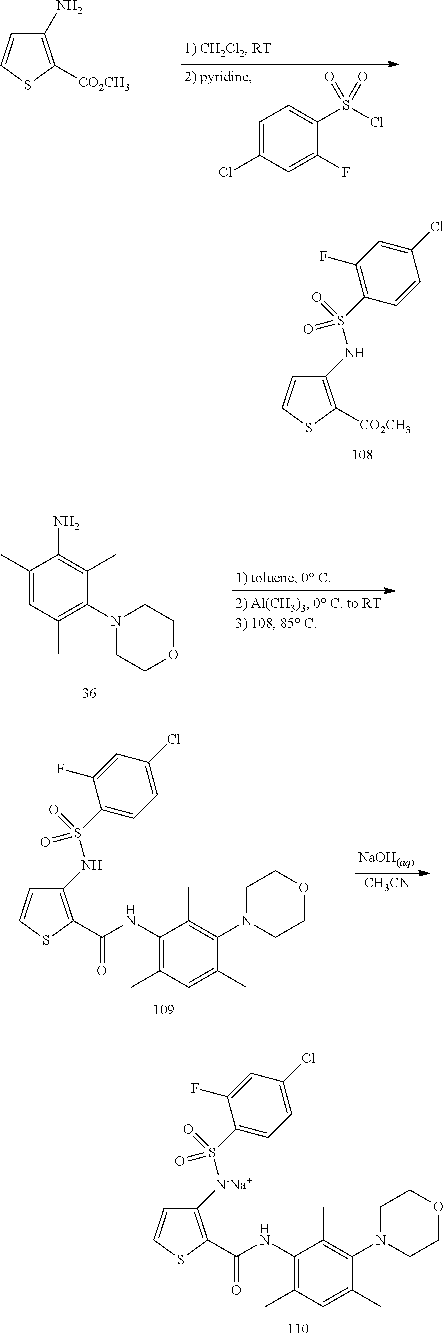

### I. Methyl 3-(4-chloro-2-fluorophenylsulfonamido)thiophene-2-carboxylate (108)

Synthesized as described for 78 using methyl 3-aminothiophene-2-carboxylate (258 mg; 1.64 mmol), 4-chloro-2-fluorobenzene-1-sulfonyl chloride (419 mg; 1.83 mmol), pyridine (0.41 mL; 5.07 mmol), and dichloromethane (2 mL). This afforded the title compound (534 mg).

### II. 3-(4-Chloro-2-fluorophenylsulfonamido)-N-[2,4,6-trimethyl-3-(morpholin-4-yl)phenyl]thiophene-2-carboxamide (109)

Synthesized as described for 85 using 36 (217 mg; 0.98 mmol), 108 (396 mg; 1.13 mmol), trimethylaluminum (2.5 mL; 5.0 mmol; 2M in hexanes) and toluene (1 mL). The resulting crude product purified by automated silica gel column chromatography (Biotage®) eluting with an ethyl acetate/hexanes gradient (8% to 80%) to give the title compound (196 mg).

### III. 3-(4-Chloro-2-fluorophenylsulfonamido)-N-[2,4,6-trimethyl-3-(morpholin-4-yl)phenyl]thiophene-2-carboxamide, Sodium Salt (110)

Synthesized as described for 80 using 109 (189 mg; 0.35 mmol), aqueous sodium hydroxide (3.6 mL; 0.38 mmol; 0.105N) and acetonitrile (6 mL). This afforded the title compound as a yellow solid (190 mg).

Mass Spectral Data (m/z): Measured (M−H)⁻=536.20; Calculated (M−H)⁻=536.09.

### Example 33

### Preparation of 3-(4-fluorophenylsulfonamido)-N-[2,4,6-trimethyl-3-(morpholin-4-yl)phenyl]thiophene-2-carboxamide, Sodium Salt (115)

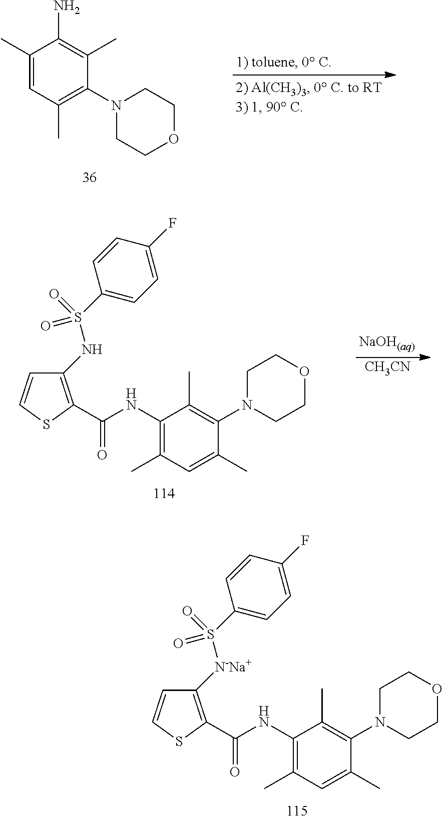

### I. 3-(4-Fluorophenylsulfonamido)-N-[2,4,6-trimethyl-3-(morpholin-4-yl)phenyl]thiophene-2-carboxamide (114)

Synthesized as described for 13 using 36 (176 mg; 0.80 mmol), toluene (5.3 mL), trimethylaluminum (1.2 mL; 2.4 mmol; 2M in hexanes) and 1 (281 mg; 0.89 mmol) and the reaction mixture was heated at 90° C. This afforded the title compound as a pale yellow solid (86 mg).

### II. 3-(4-Fluorophenylsulfonamido)-N-[2,4,6-trimethyl-3-(morpholin-4-yl)phenyl]thiophene-2-carboxamide, Sodium Salt (115)

Synthesized as described for 67 using 114 (84 mg; 0.17 mmol), aqueous sodium hydroxide (1.7 mL; 0.17 mmol; 0.1N), acetonitrile (4 mL) and water (3 mL). This afforded the title compound as a white solid (81 mg).

Mass Spectral Data (m/z): Measured (M+H)⁺=504.16; Calculated (M+H)⁺=504.14.

### Example 34

### Preparation of 3-(3,4-difluorophenylsulfonamido)-N-[2,4,6-trimethyl-3-(morpholin-4-yl)phenyl]thiophene-2-carboxamide, Sodium Salt (118)

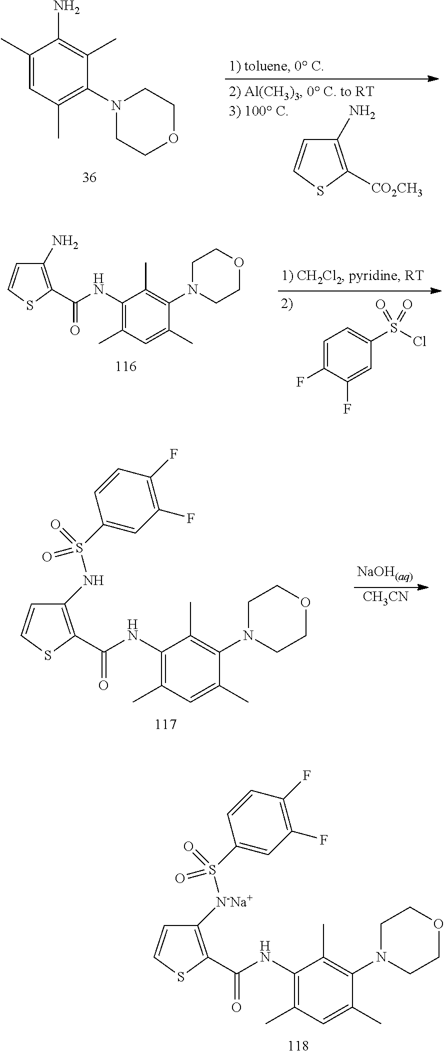

### I. 3-Amino-N-[2,4,6-trimethyl-3-(morpholin-4-yl)phenyl]thiophene-2-carboxamide (116)

Synthesized as described for 13 using 36 (1.25 g; 5.67 mmol), toluene (15 mL), trimethylaluminum (11.3 mL; 22.6 mmol; 2M in hexanes) and methyl 3-aminothiophene-2-carboxylate (1.78 g; 11.3 mmol) and the reaction mixture was heated at 100° C. The resulting crude product was purified by automated silica gel column chromatography (Biotage®) eluting with an ethyl acetate/hexanes gradient (10% to 80%) to give the title compound as a light yellow solid (0.78 g).

### II. 3-(3,4-Difluorophenylsulfonamido)-N-[2,4,6-trimethyl-3-(morpholin-4-yl)phenyl]thiophene-2-carboxamide (117)

Synthesized as described for 78 using 116 (300 mg; 0.87 mmol), 2,4-difluorobenzene-1-sulfonyl chloride (201 mg; 0.95 mmol), pyridine (0.205 g; 2.6 mmol), and dichloromethane (5 mL). The crude product was recrystallized from ethyl acetate/hexanes to give the title compound as an off-white solid (300 mg).

### III. 3-(3,4-Difluorophenylsulfonamido)-N-[2,4,6-trimethyl-3-(morpholin-4-yl)phenyl]thiophene-2-carboxamide, Sodium Salt (118)

Synthesized as described for 7 using 117 (273 mg; 0.523 mmol), aqueous sodium hydroxide (5.23 mL; 0.523 mmol; 0.1N) and acetonitrile (3 mL). This afforded the title compound as an off-white solid (285 mg).

Mass Spectral Data (m/z): Measured (M−H)⁻=520.13; Calculated (M−H)⁻=520.12.

### Example 35

### Preparation of 3-(4-tert-butylphenylsulfonamido)-N-[2,4,6-trimethyl-3-(morpholin-4-yl)phenyl]thiophene-2-carboxamide, Sodium Salt (120)

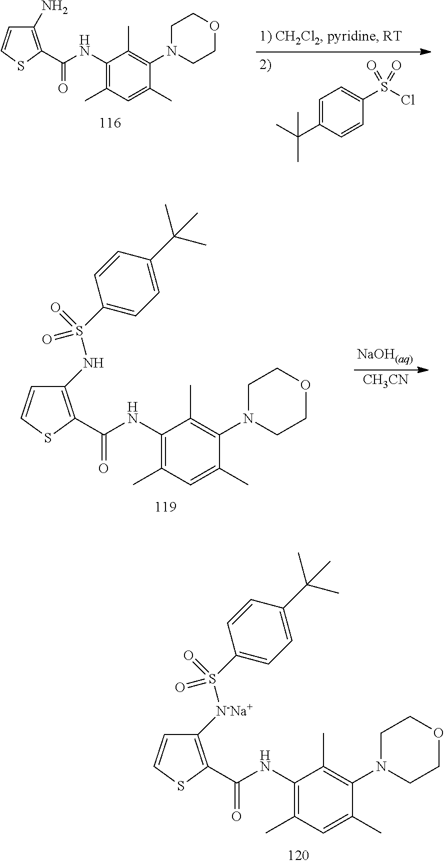

### I. 3-(4-tert-Butylphenylsulfonamido)-N-[2,4,6-trimethyl-3-(morpholin-4-yl)phenyl]thiophene-2-carboxamide (119)

Synthesized as described for 78 using 116 (100 mg; 0.29 mmol), 4-tert-butylbenzene-1-sulfonyl chloride (80 mg; 0.34 mmol), pyridine (0.068 g; 0.86 mmol), and dichloromethane (3 mL). This afforded the title compound as a white solid (86 mg).

### II. 3-(4-tert-Butylphenylsulfonamido)-N-[2,4,6-trimethyl-3-(morpholin-4-yl)phenyl]thiophene-2-carboxamide, Sodium Salt (120)

Synthesized as described for 7 using 119 (86 mg; 0.158 mmol), aqueous sodium hydroxide (1.58 mL; 0.158 mmol; 0.1N) and acetonitrile (3 mL). This afforded the title compound as a white solid (86 mg).

Mass Spectral Data (m/z): Measured (M−H)⁻=540.25; Calculated (M−H)⁻=540.20.

### Example 36

### Preparation of 3-[2-chloro-4-(trifluoromethyl)phenylsulfonamido]-N-[2,4,6-trimethyl-3-(morpholin-4-yl)phenyl]thiophene-2-carboxamide, Sodium Salt (123)

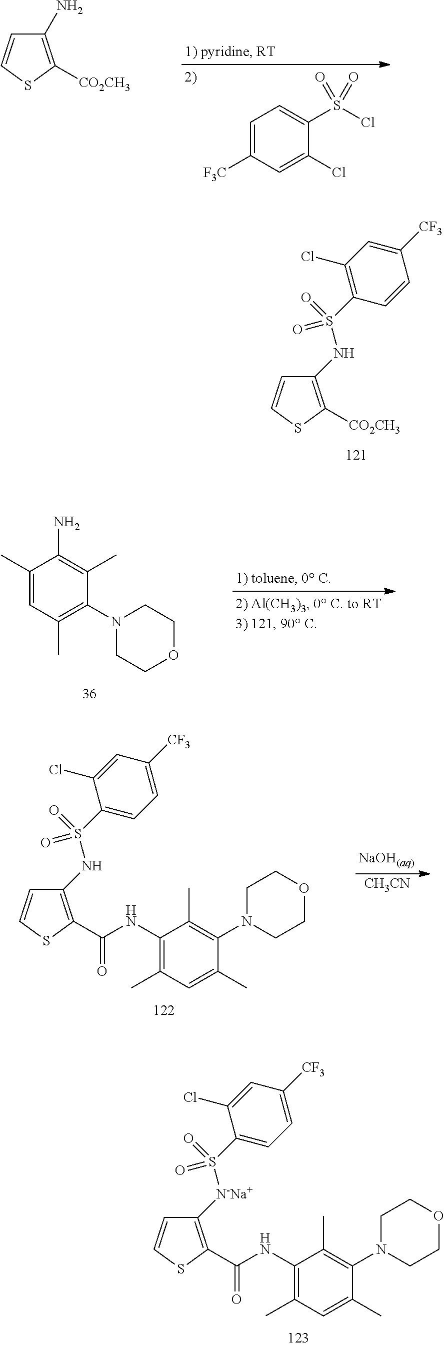

### I. Methyl 3-[2-chloro-4-(trifluoromethyl)phenylsulfonamido]thiophene-2-carboxylate (121)

Synthesized as described for 71 using methyl 3-aminothiophene-2-carboxylate (0.30 g; 1.9 mmol), 2-chloro-4-(trifluoromethyl)benzene-1-sulfonyl chloride (0.33 mL; 1.9 mmol) and pyridine. This afforded the title compound as an orange solid (0.55 g).

### II. 3-[2-Chloro-4-(trifluoromethyl)phenylsulfonamido]-N-[2,4,6-trimethyl-3-(morpholin-4-yl)phenyl]thiophene-2-carboxamide (122)

Synthesized as described for 13 using 36 (0.20 g; 0.90 mmol), toluene (5 mL), trimethylaluminum (1.9 mL; 3.8 mmol; 2M in hexanes) and 121 (0.36 g; 0.90 mmol) and the reaction mixture was heated at 90° C. The resulting crude product was purified by automated silica gel column chromatography (Biotage®) eluting with an ethyl acetate/hexanes gradient to give the title compound as an off-white solid (0.16 g).

### III. 342-Chloro-4-(trifluoromethyl)phenylsulfonamido]-N-[2,4,6-trimethyl-3-(morpholin-4-yl)phenyl]thiophene-2-carboxamide, Sodium Salt (123)

Synthesized as described for 7 using 122 (0.16 g; 0.27 mmol), aqueous sodium hydroxide (2.6 mL; 0.27 mmol; 0.105N) and acetonitrile (1 mL). This afforded the title compound as an off-white solid (0.13 g).

Mass Spectral Data (m/z): Measured (M−H)⁻=586.10; Calculated (M−H)⁻=586.09.

### Example 38

### CCR9 FLIPR/FlexStation Assay Protocol

CCR9-Flp-CHO cells were seeded at 25,000 cells/well in a clear bottom, black wall 96-well plate (Greiner #655090) one day prior to assay. Cells were grown in a tissue culture incubator at 37° C. with 5% CO₂ for 18 to 24 hours.

Wash buffer and dye loading buffer were prepared fresh each time the assay was performed. Wash buffer was prepared according to the following protocol: 20 ml 10×HBSS (Invitrogen/Gibco #14065-056), 4 ml 1 M HEPES (Sigma H3784), 174 ml sterile deionized water; then 140 mg probenecid (Sigma P8761) dissolved in 2 ml 1 M NaOH (Fisher S318) was added to solution and pH adjusted to 7.4. This wash buffer contains 1×HBSS, 20 mM HEPES and 2.5 mM probenecid. For one 96-well plate, dye loading buffer was prepared as following: 11 ml wash buffer, 44 μl Fluo-4/pluoronic acid mix (prepared from 22 μL aliquot of 2 mM Fluo-4 (Molecular Probes F14202, 1 mg/tube)+22 μl 20% pluronic F-127 (Molecular Probes P3000MP).

Cells were loaded with dye according to the protocol below:
1. Wash buffer was prepared with 1×HBSS/HEPES at room temperature
2. Loading buffer was prepared (keep in dark)
3. Culture media was aspired and plates washed with 100 μl wash buffer per well×1
4. 100 μl dye loading buffer was added to each well
5. Plates were incubated at 37° C. for 30 minutes
6. Loading buffer was aspirated
7. Plates were washed with 100 μl wash buffer per well×3
8. 100 μl wash buffer was added per well
9. Plates were incubated at 37° C. for 30 minutes
10. Plate were assayed with FLIPR or FlexStation

10 mM stock compounds in DMSO were prepared and diluted in DMSO to 1 mM. Compounds were diluted in wash buffer to make 8 point series dilutions containing same final concentration of DMSO (1%). Compounds were tested in duplicate wells for each point. Ligand rhTECK (R&D Systems 334-TK) was diluted to 6× of its EC70 with wash buffer. Appropriate amount of 6× ligand was added to each well. Data was analyzed using XLfit3 software to calculate IC₅₀ value of antagonist activity for each compound.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

What is claimed is:

1. A compound of Formula I: 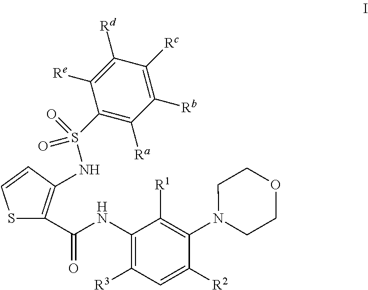
or a pharmaceutically acceptable derivative thereof,
wherein R¹ and R³ are each alkyl; R² is selected from halo and alkyl; and
R^{a}, R^{b}, R^{c}, R^{d} and R^{e} are each independently selected from hydrogen, halo, alkyl, alkoxy, haloalkyl and haloalkoxy, with a proviso that when R^{a}, R^{b}, R^{c}, R^{d} and R^{e} are each hydrogen then at least one of R¹, R² or R³ is other than methyl.

2. A compound according to claim 1 of formula: 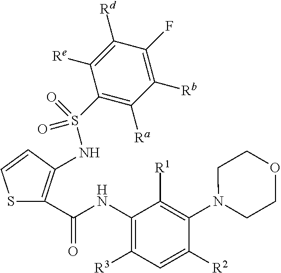
or a pharmaceutically acceptable derivative thereof.

3. A compound according to claim 1 of formula: 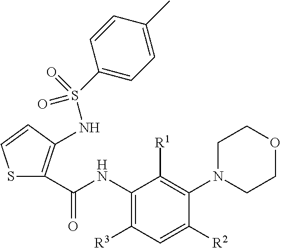
or a pharmaceutically acceptable derivative thereof.

4. A compound according to claim 1 of formula: 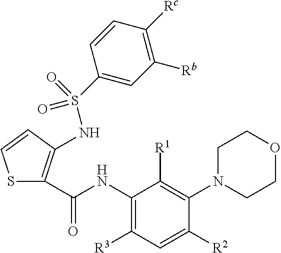
or a pharmaceutically acceptable derivative thereof.

5. A compound according to claim 1 of formula: 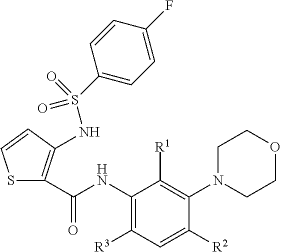
or a pharmaceutically acceptable derivative thereof.

6. A compound according to claim 1 of formula: 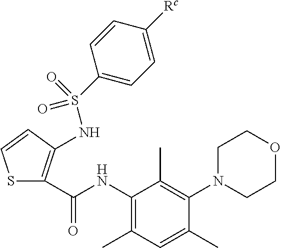
or a pharmaceutically acceptable derivative thereof.

7. A compound according to claim 1 selected from: 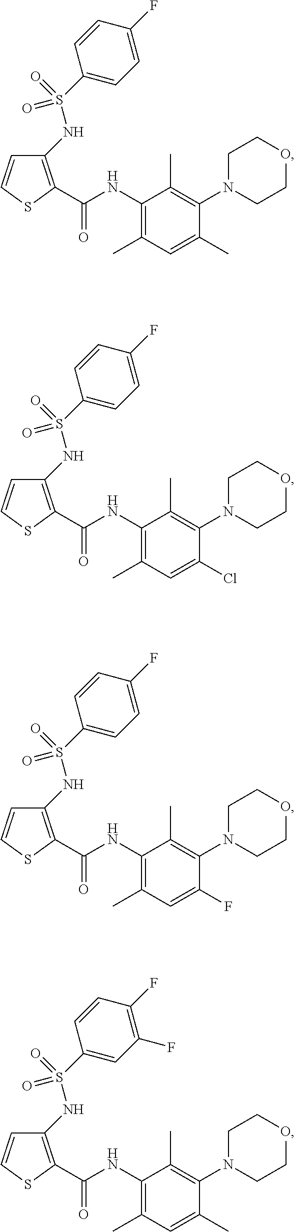 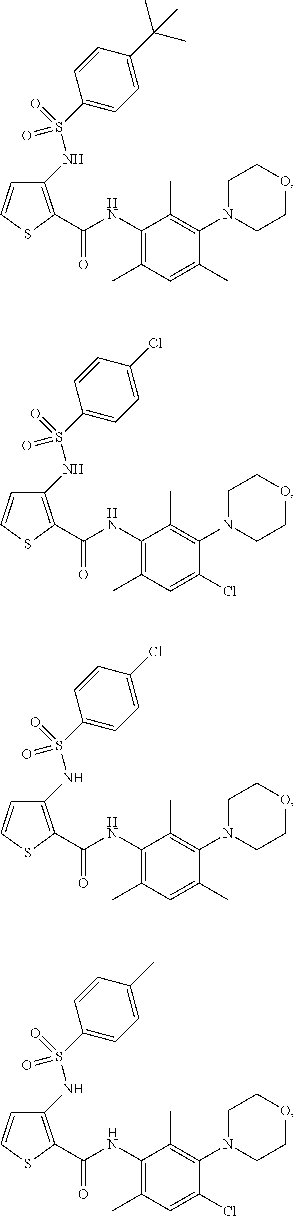 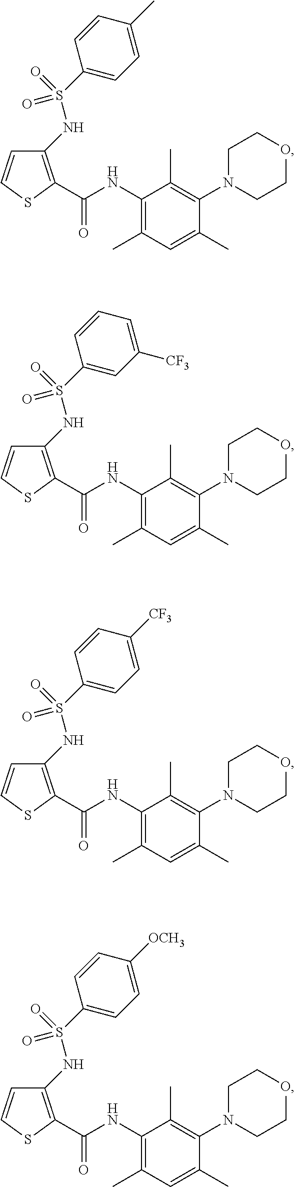 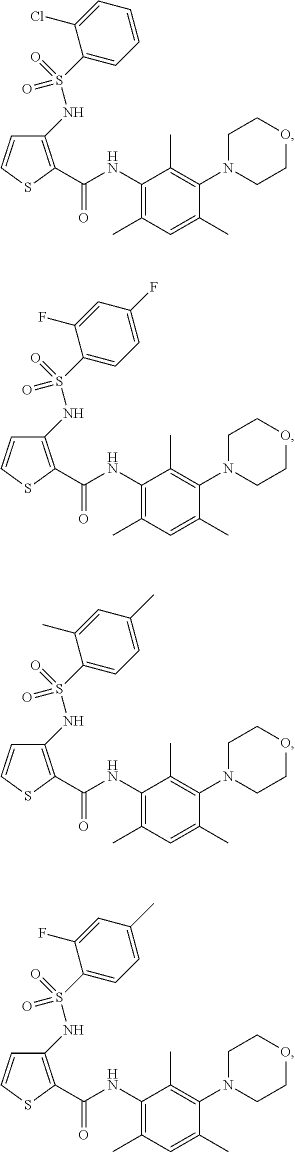 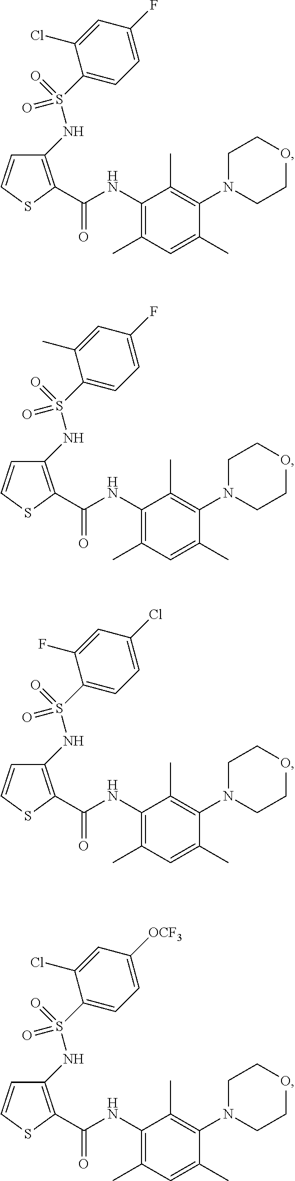 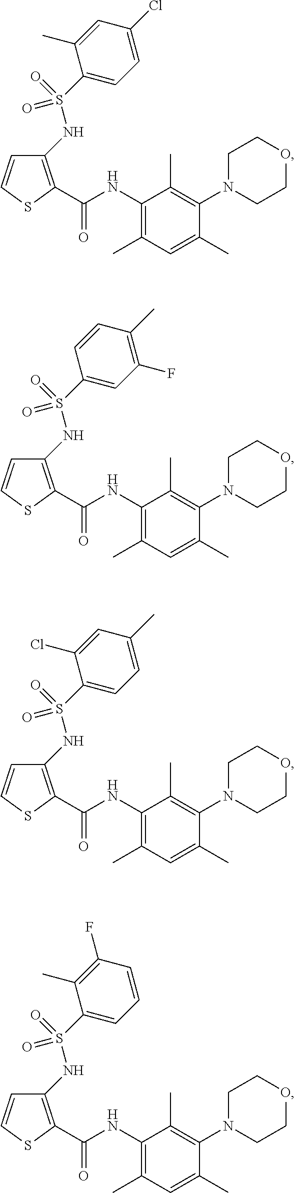 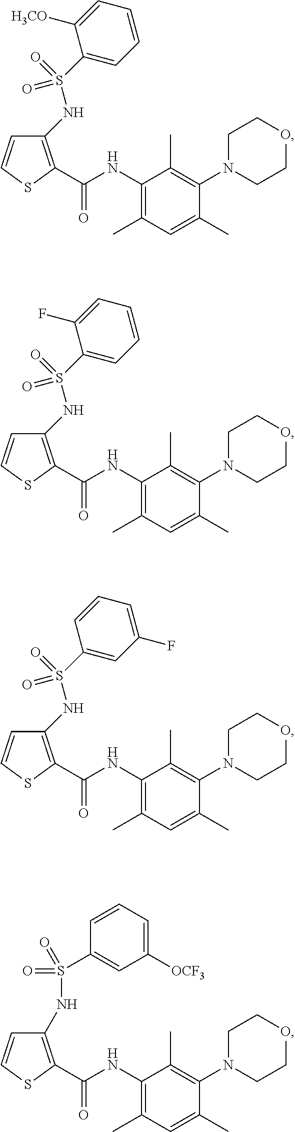 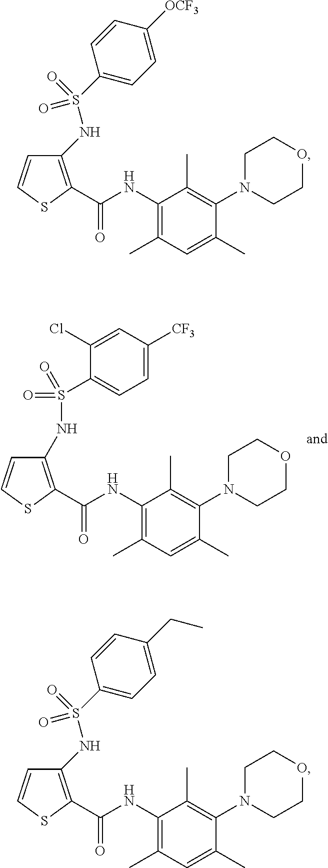
or a pharmaceutically acceptable derivative thereof.

8. A compound of Formula II: 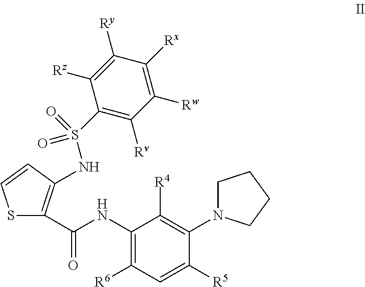
or a pharmaceutically acceptable derivative thereof,
wherein R⁴ is alkyl; R⁵ and R⁶ are each independently selected from halo and alkyl such that at least one of R⁵ or R⁶ is halo;
R^{v}, R^{w}, R^{x}, R^{y} and R^{z} are each independently selected from hydrogen, halo and alkyl.

9. A compound according to claim 8 of formula: 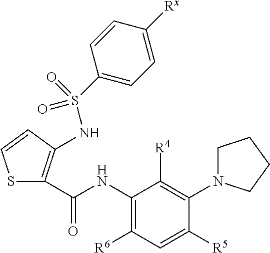
or a pharmaceutically acceptable derivative thereof.

10. A compound according to claim 8 of formula: 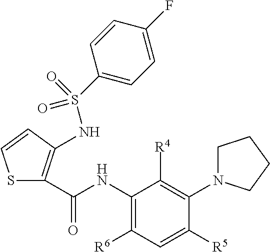
or a pharmaceutically acceptable derivative thereof.

11. A compound according to claim 8 selected from

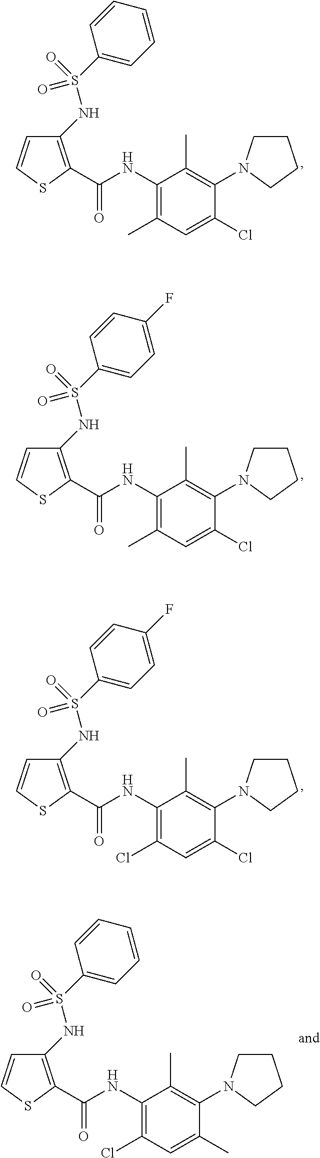

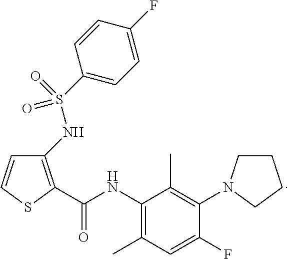

12. A pharmaceutical composition comprising a compound of any of claims 1-11 or a pharmaceutically acceptable derivative thereof, and a pharmaceutically acceptable carrier.

13. A method for modulating CCR9 receptor activity comprising contacting the CCR9 receptor with the compound of any of claims 1-11 or a pharmaceutically acceptable derivative thereof.

14. A method for treating, preventing or ameliorating a disease associated with CCR9 receptor modulation comprising administering the compound of any of claims 1-11 or a pharmaceutically acceptable derivative thereof.

15. The method of claim 14, wherein the disease is Crohn's disease, ulcerative colitis, celiac disease, primary sclerosing cholangitis, HIV, prostate cancer, leukemia, small intestinal cancer or melanoma.
